(19) United States
(12) Patent Application Publication
(10) Pub. No.: US 2017/0227553 A1
(43) Pub. Date: Aug. 10, 2017

(54) COMPOSITIONS AND METHODS FOR IDENTIFYING AND TREATING CONDITIONS INVOLVING HSF1 ACTIVITY

(71) Applicant: Duke University, Durham, NC (US)

(72) Inventors: Dennis J. Thiele, Durham, NC (US); Rocio Gomez-Pastor, Durham, NC (US); Daniel W. Neef, Durham, NC (US)

(21) Appl. No.: 15/501,765

(22) PCT Filed: Aug. 4, 2015
(86) PCT No.: PCT/US2015/043573

§ 371 Date: Feb. 3, 2017

Related U.S. Application Data
(60) Provisional application No. 62/033,002, filed on Aug. 4, 2014.

(51) Int. Cl. G01N 33/68; A61K 31/404; A61K 31/501; A61K 31/4184; A61K 31/122; A61K 31/4035

(52) U.S. Cl. CPC G01N 33/6896; A61K 31/122; A61K 31/4035; A61K 31/501; A61K 31/4184; A61K 31/404; G01N 2800/52; G01N 2440/14

(57) ABSTRACT

Provided herein are methods for identifying and treating subjects having conditions involving reduced HSF1 activity (e.g., diminished HSF1 activity, diminished HSF1 protein levels, increased HSF1 Ser303 phosphorylation, increased HSF1 Ser307 phosphorylation) or conditions that benefit from increasing HSF1 abundance or activity beyond physiological levels.

### FIELD OF THE INVENTION

Provided herein are methods for identifying and treating subjects having conditions involving reduced HSF1 activity (e.g., diminished HSF1 activity, diminished HSF1 protein levels, increased HSF1 Ser303 phosphorylation, increased HSF1 Ser307 phosphorylation) or conditions that benefit from increasing HSF1 abundance or activity beyond physiological levels.

### BACKGROUND OF THE INVENTION

Proteopathies are human diseases caused by protein misfolding, which causes cellular stress sensitivity, dysfunction and death. Proteopathies can occur in neuronal tissue (typical of neurodegenerative diseases) or in a variety of other tissues including, but not limited to heart, muscle, spleen and liver. Examples of proteopathies include, but are not limited to: Alzheimer's disease, glaucoma, prion disease, tauopathies, fronto-temporal degeneration, Huntington's disease, Kennedy's disease, familial dementia, Cushing's disease, neurofibromatosis, some lysosomal storage diseases, diabetes, cataracts, cardiac atrial amyloidosis, Parkinson's disease, cystic fibrosis, sickle cell disease and others.

Neurodegenerative diseases including Alzheimer's, Parkinson's, Amyotropic Lateral Sclerosis, Huntington's, transmissible spongiform encephalopathy and others exhibit common features that include protein misfolding, cellular stress and dysfunction, inflammation and neuronal cell death. While there are currently very limited therapeutic approaches for these devastating diseases, understanding the fundamental mechanisms by which cells cope with the stress of protein misfolding and aggregation, and the defects in mounting stress-protective responses to maintain neuronal function and viability in the disease state, may lead to the identification of new therapies. Currently, 14 neurodegenerative disorders are known to be caused by tandem amplification of tri-nucleotide repeats encoding glutamine (Q) within protein coding regions. For example, Huntington's disease (HD) results from a polyQ repeat expansion in exon 1 of the HTT gene encoding Huntingtin. In HD, progressive neurodegeneration, primarily in the striatal and cortical neurons, is associated with protein misfolding and aggregation as a consequence of the elongated polyQ tracks in Htt protein.

In addition to protein misfolding underlying many neurodegenerative diseases, there are a number of other diseases caused by protein misfolding, which together are called proteopathies. Other protein conformational disorders, or protein folding disorders, include but are not limited to retinal ganglion cell degeneration in glaucoma, prion disease, multiple tauopathies, Kennedy's disease, familial British dementia, familial Danish dementia, Alexander Disease, Serpinopathies, light chain or heavy chain amyloidosis, Type II diabetes, aortic medial amyloidosis, Glycogen Storage Disease type IV (Andersen Disease), cataracts, cardiac atrial amyloidosis, atrial fibrillation, Crohn's Disease, Autosomal Dominant Hyper-IgE Syndrome, and others.

Improved methods for identifying and treating proteopahthies are needed.

### BRIEF SUMMARY OF THE INVENTION

The Heat Shock Transcription Factor 1 (HSF1) is a major stress-responsive transcription factor in eukaryotic cells that protects cells from stress-induced dysfunction and death. HSF1 protects cells from stressful conditions caused by protein misfolding by activating the transcription of a plethora of target genes that encode proteins that function in protein folding and quality control, preventing apoptosis and in cell survival (FIG. 1). The pathological phenotypes of cellular and animal models of protein misfolding disease are further aggravated by the observations that expression levels of several HSF1 target genes encoding protein chaperones and anti-apoptotic proteins may be decreased, thus increasing cellular stress, dysfunction and apoptosis. Moreover, a mouse model of Spinal and Bulbar Muscular Atrophy (SBMA) resulting from a polyQ repeat in the Androgen Receptor (AR-polyQ), showed an inverse correlation between HSF1 levels in the CNS and the abundance of AR-polyQ inclusion bodies and SBMA mice heterozygous for HSF1 exhibited increased AR-polyQ aggregates in neurons, aggregates in non-neuronal tissues and enhanced neurodegeneration. In a complementary study, expression of a constitutively active form of HSF1 lacking the central regulatory domain that encompasses the Serine303 residue, in the R6/2 mouse model of Huntington's Disease, inhibited inclusion body formation and prolonged lifespan.

HSF1 is the master activator of chaperone gene expression and it coordinately activates the transcription of genes that encode protein chaperones that are required for protein folding. Protein chaperones play critical roles in the folding, modification, maturation and degradation of cellular client proteins. With respect to cell survival, chaperones stabilize cellular pro-survival proteins such as AKT kinase and inhibit the activation of pro-death proteins such as JNK kinase. Experiments in cellular, fruit fly, worm and mouse models of neurodegenerative disease and in other protein aggregation diseases demonstrate that increased expression of HSF1 target genes ameliorates protein aggregation and cell death. As protein chaperones function in hetero-multimeric complexes, co-expression of distinct chaperones such as Hsp70 and Hsp40 synergize in cellular stress protection. Clearly, human HSF1 is an important target for the development of therapies for proteopathies.

Experiments conducted during the course of developing embodiments for the present invention identified a reduction in HSF1 protein levels in Huntington's Disease patients and in cellular and mouse models of Huntington's Disease (HD) and other poly-glutamine based disease. Moreover, decreased levels of HSF1 were shown to correlate with increased levels of HSF1 phosphorylation at Serine residues 303 and 307 in HSF1. It was further shown that RNAi-mediated knock-down of Casein Kinase II (CK2) subunits (or through the use of Casein Kinase II enzyme inhibitors) in cell culture models of polyQ-based disease, HSF1 levels are elevated, HSF1 Ser303 and 307 phosphorylation is reduced, HSF1 activity is elevated, the expression of protein chaperone genes and other HSF1 target genes is elevated, and that cells are protected from stress-induced cell death. Moreover, experiments were conducted showing CK2 inhibiting activity for specific compounds (see, Example 8 and FIG. 54).

Accordingly, provided herein are methods for identifying and treating subjects having conditions involving aberrant HSF1 activity through inhibiting CK2 activity. In addition, the present invention contemplates that CK2 inhibitors have therapeutic benefit by increasing HSF1 protein levels and/or activity beyond normal physiological levels. Moreover, the present invention contemplates that CK2 inhibitors have therapeutic benefit by increasing HSF1 protein levels and/or activity when HSF1 protein levels or activity are lower than normal.

In certain embodiments, the present invention provides methods of treating a patient having a condition ameliorated by elevation of HSF1 levels or HSF1 activity in cells, the method comprising administering a therapeutically effective amount of a CK2 inhibitor to the patient.

In certain embodiments, the present invention provides methods of selecting a patient having a condition ameliorated by elevation of HSF1 levels with a CK2 inhibitor, the method comprising obtaining a biological sample from the patient; determining whether the biological sample contains cells having aberrant HSF1 activity (e.g., diminished HSF1 levels, diminished Hsp70 levels, increased HSF1 Ser303 phosphorylation, increased HSF1 Ser307 phosphorylation); and selecting the patient for treatment if the biological sample contains cells having aberrant HSF1 activity. In some embodiments, the methods further comprise administering a therapeutically effective amount of the CK2 inhibitor to the patient.

In certain embodiments, the present invention provides methods of predicting treatment outcome in a patient having a condition ameliorated by elevation of HSF1 levels, the method comprising: obtaining a biological sample from the patient; and determining whether the biological sample contains cells having aberrant HSF1 activity (e.g., diminished HSF1 activity, diminished levels of HSF1 protein, increased HSF1 Ser303 phosphorylation, increased HSF1 Ser307 phosphorylation); wherein the detection of cells having aberrant HSF1 activity indicates that administering a therapeutically effective amount of a CK2 inhibitor to the patient will cause a favorable therapeutic response.

In certain embodiments, the present invention provides methods of treating a patient having a condition ameliorated by elevation of HSF1 levels, the method comprising obtaining a biological sample from the patient (e.g. muscle biopsy); determining whether the biological sample contains cells having aberrant HSF1 activity (e.g., diminished HSF1 levels, diminished Hsp 70 levels, increased HSF1 Ser303 phosphorylation, increased HSF1 Ser307 phosphorylation); and administering a therapeutically effective amount of a CK2 inhibitor to the patient if the biological sample contains cells having aberrant HSF1 activity.

Such methods are not limited to a particular type of biological sample. In some embodiments, the biological sample comprises muscle cells and/or neurological cells.

Such methods are not limited to a particular type of patient. In some embodiments, the patient is human.

Such methods are not limited to a particular condition ameliorated by elevation of HSF1 activity in cells. In some embodiments, the condition ameliorated by restoration of HSF1 activity is selected from the group consisting of dentatorubropallidoluysian atrophy, Huntington's Disease, spinobulbar muscular atrophy (Kennedy disease), spinocerebellar ataxia type 1, spinocerebellar ataxia type 2, spinocerebellar ataxia type 3 (Machado-Joseph disease), spinocerebellar ataxia type 6, spinocerebellar ataxia type 7, spinocerebellar ataxia type 17, fragile X syndrome, fragile X-associated tremor/ataxia syndrome), fragile XE mental retardation, Friedreich's ataxia, myotonic dystrophy, spinocerebellar ataxia type 8, and spinocerebellar ataxia type12. In some embodiments, the condition ameliorated by elevation of HSF1 levels is selected from the group consisting of Alzheimer's disease, glaucoma, prion disease, tauopathies, fronto-temporal degeneration, Huntington's disease, Kennedy's disease, familial dementia, Cushing's disease, neurofibromatosis, some lysosomal storage diseases, diabetes, cataracts, cardiac atrial amyloidosis, Parkinson's disease, cystic fibrosis, and sickle cell disease. In some embodiments, the condition ameliorated by restoration of HSF1 activity is any type of proteopathy disorder. Examples of a proteopathy disorder include, but are not limited to, Alzeimer's disease, cerebral β-amyloid angiopathy, retinal ganglion cell degeneration in glaucoma, prion diseases, Parkinson's disease, synucleinopathies, tauopahties, frontotemporal lobar degeneration, amyotrophic lateral sclerosis, Huntington's disease, hereditary cerebral hemorrhage with amyloidosis, cerebral autosomal-dominant arteriopathy with subcortical infarcts and leukoencephalopathy, Alexander disease, seipinopathies, familial amyloidotic neuropathy, senile systemic amyloidosis, serpinopathies, light chain amyloidosis, heavy chain amyloidosis, secondary amyloidosis, type 2 diabetes, aortic medial amyloidosis, ApoAI amyloidosis, ApoAII amyloidosis, ApoAIV amyloidosis, familial amyloidosis of the Finnish type, lysozyme amyloidosis, fibrinogen amyloidosis, dialysis amyloidosis, inclusion body myositis/myopathy, cataracts, retinitis pigmentosa with rhodopsin mutations, medullary thyroid carcinoma, cardiac atrial amyloidosis, pituitary prolactinoma, hereditary lattice corneal dystrophy, cutaneous lichen amyloidosis, Mallory bodies, corneal lactoferrin amyloidosis, pulmonary alveolar proteinosis, odontogenic (Pinborg) tumor amyloid, seminal vesical amyloid, cystric fibrosis, sickle cell disease, and critical illness myopathy.

Such methods are not limited to a particular type of CK2 inhibitor. In some embodiments, the CK2 inhibitor is selected from the group consisting of TID43, Emodin, TBBz, 7,8-dichloro-1,4-dihydro-4-oxo-3-quinolinecarboxylic acid (Cay10577), resorufin, TBCA, Quinalizarin, AZ285, TID43, TBB, CX-4945, CX-5011, CX-5279, TBI, TBCA, DMAT, CIBG-300, Ellagic Acid, K64/PBIN, K66/TMCB, IQA, Fisetin, Hematein, FLC21, TID46, Quinolone 9, Quinolone 7, TTP22, FNH79, 7-Amino-5-(4-methoxyphenyl)pyrazolo[1,5-a]pyrimidine-3-carbonitrile, 7-(Cyclopropylamino)-5-(2-thienyl)pyrazolo[1,5-a]pyrimidine-3-carbonitrile, 7-(Cyclopropylamino)-5-(4-methoxyphenyl)pyrazolo[1,5-a]pyrimidine-3-carbonitrile, 5-(4-Methoxyphenyl)-7-[(1-methyl-1H-pyrazol-3-yl)amino]pyrazolo[1,5-a]pyrimidine-3-carbonitrile, 7-(Cyclopropylamino)-5-(6-methoxypyridin-3-yl)pyrazolo[1,5-a]pyrimidine-3-carbonitrile, N-(1-(3-cyano-7-(cyclopropylamino)pyrazolo[1,5-a]pyrimidin-5-yl)-1H-indol-6-yl)acetamide, N-(1-(3-cyano-7-(cyclopropylamino)pyrazolo[1,5-a]pyrimidin-5-yl)-1H-indazol-6-yl)acetamide, N-[1-[3-cyano-7-(cyclopropylamino)pyrazolo[1,5-a]pyrimidin-5-yl]pyrrolo[3,2-b]pyridin-6-yl]acetamide, N-[1-[3-cyano-7-(cyclopropylamino)pyrazolo[1,5-a]pyrimidin-5-yl]indolin-6-yl]acetamide, N-{4-[3-cyano-7-(cyclopropylamino)pyrazolo[1,5-a]pyrimidin-5-yl]-3,4-dihydro-2H-1,4-benzoxazin-6-yl}acetamide, N-(1-(3-cyano-7-(cyclopropylamino)pyrazolo[1,5-a]pyrimidin-5-yl)-1,2,3,4-tetrahydroquinolin-7-yl)acetamide, N-(1-(3-cyano-7-(cyclopropylamino)pyrazolo[1,5-a]pyrimidin-5-yl)-5-methyl-1H-indol-6-yl)acetamide, N-(1-(3-cyano-7-(1-methyl-1H-pyrazol-3-ylamino)pyrazolo[1,5-a]pyrimidin-5-yl)-1H-indol-6-yl)acetamide, N-(1-(3-cyano-7-(oxetan-3-ylamino)pyrazolo[1,5-a]pyrimidin-5-yl)-1H-indol-6-yl)acetamide, N-(1-(3-cyano-7-(4-hydroxybutylamino)pyrazolo[1,5-a]pyrimidin-5-yl)-1H-indol-6-yl)acetamide, N-(1-(3-cyano-7-(2-morpholinoethylamino)pyrazolo[1,5-a]pyrimidin-5-yl)-1H-indol-6-yl)acetamide, N-(1-(3-cyano-7-(2-(pyrrolidin-1-yl)ethylamino)pyrazolo[1,5-a]pyrimidin-5-yl)-1H-indol-6-yl)acetamide, N-(1-(3-Cyano-7-(3-(dimethylamino)propylamino)pyrazolo[1,5-a]pyrimidin-5-yl)-1H-indol-6-yl)acetamide, N-(1-(3-Cyano-7-(3-(pyrrolidin-1-yl)propylamino)pyrazolo[1,5-a]pyrimidin-5-yl)-1H-indol-6-yl)acetamide, N-(1-(3-Cyano-7-(cyclopropylamino)pyrazolo[1,5-a]pyrimidin-5-yl)-1H-indol-6-yl)-N-methylacetamide, 7-(Cyclopropylamino)-5-(6-(hydroxymethyl)-1H-indol-1-yl)pyrazolo[1,5-a]pyrimidine-3-carbonitrile, 7-(cyclopropylamino)-5-(6-(methylsulfonylmethyl)-1H-indol-1-yl)pyrazolo[1,5-a]pyrimidine-3-carbonitrile, N-{3-[3-cyano-7-(cyclopropylamino)pyrazolo[1,5-a]pyrimidin-5-yl]-1-methyl-1H-indol-5-yl}acetamide, N-(3-(3-cyano-7-(cyclopropylamino)pyrazolo[1,5-a]pyrimidin-5-yl)-1H-indol-5-yl)acetamide, N-(3-(3-cyano-7-(cyclopropylamino)pyrazolo[1,5-a]pyrimidin-5-yl)-1-(2-hydroxyethyl)-1H-indol-5-yl)acetamide, N-(3-(3-cyano-7-(cyclopropylamino)pyrazolo[1,5-a]pyrimidin-5-yl)-1-(3-hydroxypropyl)-1H-indol-5-yl)acetamide, Methyl 3-(5-acetamido-3-(3-cyano-7-(cyclopropylamino)pyrazolo[1,5-a]pyrimidin-5-yl)-1H-indol-1-yl)propanoate, 3-(5-Acetamido-3-(3-cyano-7-(cyclopropylamino)pyrazolo[1,5-a]pyrimidin-5-yl)-1H-indol-1-yl)propanoic acid, 7-(cyclopropylamino)-5-(6-nitro-1H-indazol-1-yl)pyrazolo[1,5-a]pyrimidine-3-carbonitrile, 5-(6-Amino-1H-indazol-1-yl)-7-(cyclopropylamino)pyrazolo[1,5-a]pyrimidine-3-carbonitrile, CX-4945, CX-5011, CX-5279, CX-5033 (Kang, et al., 2009 PLoS One 4(8):e6611), 5-oxo-5,6-dihydroindolo-(1,2-a)quinazolin-7-yl]acetic acid (IQA), 4,5,6,7-tetrabromobenzotriazole (TBB), CX-8184, 5-oxo-5,6-dihyroindolo[1,2-a]quinazolin-7-yl)acetic acid, myricetin, quercetin, fisetin, kaempferol, apigenin, any of the compounds shown in FIGS. 12-45,

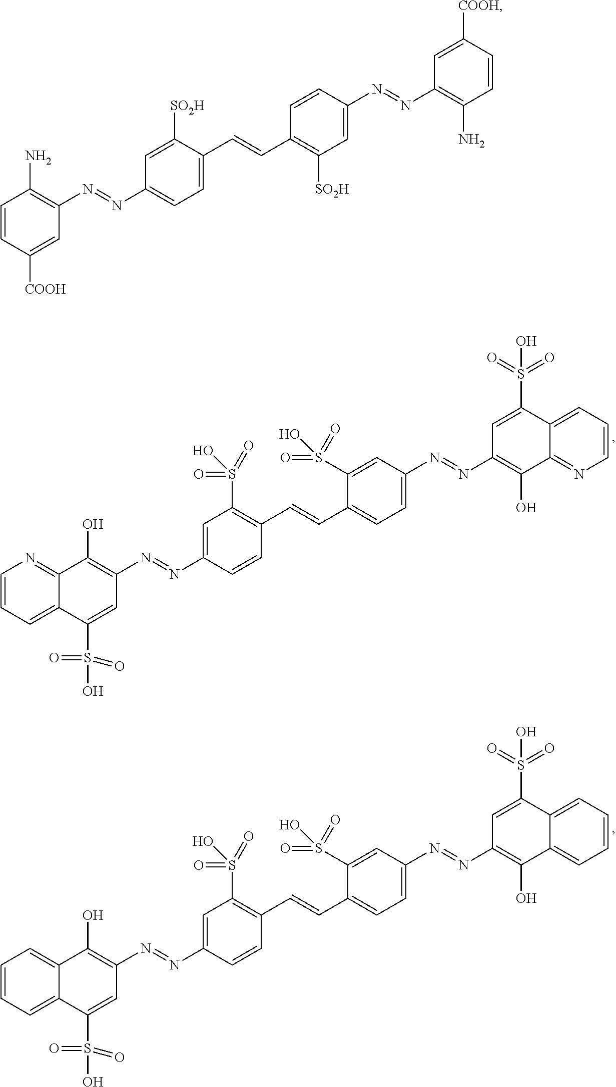

tetrahalogenobenzimidazoles, 4,5,6,7-tetrabromo- and 4,5,6,7-tetraiodo-1H-benzimidazoles, and N¹- and 2-S-carboxyalkyl derivatives.

In some embodiments, the CK2 inhibitor targets the CK2-α and/or the CK2α′ subunit of CK2 within cells having diminished HSF1 activity.

In some embodiments, the CK2 inhibitor is shown in FIG. 54 and described in Example 8.

In certain embodiments, the present invention provides methods of treating a patient having Alzheimer's Disease, the method comprising administering a therapeutically effective amount of a CK2 inhibitor to the patient. In some embodiments, administration of the CK2 inhibitor results in one or more of increased HSF1 protein levels, increased HSF1 activity, increased Hsp70 expression levels, decreased HSF1 Ser303 phosphorylation, and decreased HSF1 Ser307 phosphorylation levels. In some embodiments, the effectiveness of the treatment and the HSF1 protein level elevation can be detected by testing the HSF1 and/or Hsp70 protein levels in a muscle biopsy taken from the patient. In some embodiments, the patient is human.

In certain embodiments, the present invention provides methods of treating a patient having Huntington's Disease, the method comprising administering a therapeutically effective amount of a CK2 inhibitor to the patient. In some embodiments, administration of the CK2 inhibitor results in one or more of increased HSF1 protein levels, increased HSF1 activity, increased Hsp70 protein levels, decreased Htt-Q74 protein aggregation, enhanced cell viability, decreased HSF1 Ser303 phosphorylation, and decreased HSF1 Ser307 phosphorylation levels. In some embodiments, the effectiveness of the treatment and the HSF1 protein level elevation can be detected by testing the HSF1 and/or Hsp70 protein levels in a muscle biopsy taken from the patient. In some embodiments, the patient is human.

### BRIEF DESCRIPTION OF THE DRAWINGS

FIG. 1 shows that human HSF1 is a stress-activated transcription factor that is stress-protective. Generally, in the cytoplasm of human and other mammalian cells HSF1 exists predominantly as inactive monomer. In response to protein misfolding and other stressful conditions HSF1 is converted to a homo-trimeric species, which accumulates in the cell nucleus, binds target gene promoters and activates target gene transcription. Proteins encoded by HSF1 target genes protect cells by virtue of their activity, either directly or indirectly, in protein folding and quality control mechanisms, in the prevention of apoptosis (programmed cell death), in the reduction of inflammation and through a number of other biological functions.

FIG. 2 shows Heat Shock Transcription Factor 1 (HSF1) activation of target gene expression is impaired in the polyQ expressing rat pheochromocytoma cell line PC12. (A) Rat pheochromocytoma PC12 cells expressing the pathogenic Htt-Q74 protein were either not treated (−Dox) or treated with doxycycline at 1 μg/mL for 3 days (+Dox) to induce polyglutamine protein expression. Cells were incubated at 37° C. (C) or heat shocked at 42° C. (1 h) followed by 7 h recovery (HS). Total protein was analyzed by SDS-PAGE and immunoblotting for several Heat Shock Proteins (Hsps) using GFP as control for Htt-Q74 induction and GAPDH as a protein loading control. Hsps analyzed were: Hsp70, Hsp25, Bag3 and Hspb5 (B) Samples from (A) were quantitatively analyzed for inducible Hsp70 expression by ELISA. Data are presented as means±SE from three separate experiments (***P<0.001).

FIG. 3 shows HSF1 DNA binding and transcriptional activity is impaired in polyQ expressing cells. (A) Htt-Q74 expressing PC12 cells were either not treated (−Dox) or treated with doxycycline at 1 μg/mL during 3 days (+Dox). Cells were incubated at 37° C. (C) or heat shocked at 42° C. (1 h) followed by 1 h, 3 h, 7 h or 24 h recovery (HS) at 37° C. mRNA expression levels for Hsp70 and Hsp25 were analyzed by qRT-PCR and values were normalized to GAPDH expression and compared to non-treated control conditions. (B) HSF1 DNA binding to target gene promoters was analyzed by Chromatin Immuno-Precipitation (ChIP). ChIP analysis of HSF1 and SP1 transcription factor binding to the Hsp70 promoter in uninduced (Q74) and induced cells (Q74+Dox) cultured at 37° C. or heat shocked at 42° C. for 1 h. Data are presented as mean±SE from three separate experiments (** P<0.01, ***P<0.001).

FIG. 4 shows cellular polyglutamine protein expression results in decreased HSF1 protein levels, increased HSF1 S303 phosphorylation and decreased Hsp70 expression during protein misfolding conditions. Htt-Q74 PC12 cells were either not treated (−Dox) or treated with doxycycline at 1 μg/mL during 3 days (+Dox). Cells were heat shocked at 42° C. (1 h) and samples were taken immediately (0 h) or following a 3 h, 5 h 7 h or 15 h recovery at 37° C. Total protein was analyzed by SDS-PAGE and immunoblotting for HSF1 and the abundance of the repressive HSF1 serine-303 phosphorylation (HSF1-S303-P) as well as for Hsp70 and GAPDH protein levels.

FIG. 5 shows reduction in HSF1 protein levels, reduced HSF1 target gene activation and increased HSF1 phosphorylation at Ser303 and Ser307 are dependent on the cellular expression of pathological polyQ repeat protein. PC12 cells expressing the non-pathogenic Htt-Q23 protein, or the pathogenic Htt-Q74 protein were either not treated (−Dox) or treated with doxycycline at 1 μg/mL during 3 days (+Dox) at 37° C. Cells were maintained at 37° C. (C) or heat shocked at 42° C. (1 h) followed by 7 h recovery (HS). Total protein was analyzed by SDS-PAGE and immunoblotting for HSF1, HSF1-Ser303 and HSF1-5307 phosphorylation, Hsp70, Hsp25, GFP and GAPDH.

FIG. 6 shows a spinal-bulbar muscular atrophy (SBMA, Kennedy's Disease) polyQ-related disease cell model showing decreased HSF1 protein levels, decreased HSF1 target gene activation and increased HSF1 S303 phosphorylation. A Rat pheochromocytoma PC12 cell model of Kennedy's disease (SBMA) expressing (A) the non-pathogenic ARQ10 protein version of the androgen receptor or (B) the pathogenic ARQ112 polyglutamine repeat protein were either not treated (−Dox) or treated with doxycycline at 1 μg/mL during 3 days (+Dox). Cells were incubated at 37° C. or heat shocked at 42° C. (1 h) followed by 7 h recovery (HS) at 37° C. Total protein was analyzed by SDS-PAGE and immunoblotting for the abundance of Hsp70, Hsp25, HSF1 and HSF1-5303 phosphorylation and poly-glutamine-androgen receptor abundance using GAPDH as loading control. Data from non-pathogenic ARQ10 and the pathogenic ARQ112 are shown separately, but are from the same blot.

FIG. 7 demonstrates that a Parkinson's disease cell model does not show HSF1 target gene activation impairment. Rat pheochromocytoma PC12 cell model of the Parkinson's disease expressing alpha-synuclein protein fused to GFP under Dox induction were either not treated (−Dox) or treated with doxycycline at 1 μg/mL during 3 days (+Dox) to induce the expression of alpha-synuclein-GFP fusion protein. Cells were incubated at 37° C. (C) or heat shocked at 42° C. (1 h) followed by 7 h recovery (HS) at 37° C. Total protein was analyzed by SDS-PAGE and immunoblotting for Hsp70, GFP as a marker for alpha-synuclein expression and GAPDH as loading control.

FIG. 8 demonstrates that HSF1 protein levels are decreased, and HSF1 S303 phosphorylation increased in the striatum from the brain a Huntington's Disease mouse model. (A) Striatal brain sections from age-matched control and Htt-Q175 gene Knock-in mice zQ175 (KI175) at day 37 after birth were subjected to immunohistochemistry analysis. Tissue sections were incubated with anti-HSF1 (green), anti-HSF1 S303 phospho (red) antibody and DAPI (blue) as control for nuclear DNA. (B) The percentage of cells in the striatum that were positively stained with anti-HSF1 was normalized versus total cells (DAPI staining) as a measure for HSF1 protein abundance. (C) The ratio between the number of cells that were positively stained with anti-HSF1-S303 phospho antibody and the HSF1 positive cells was calculated and compared to that from control mice. Data are presented as means±SE from three separate experiments using mice (n=4) for each experiment (* P<0.05, ** P<0.01).

FIG. 9 demonstrates that HSF1 levels is specifically decreased in Huntington's disease but not in ALS and Parkinson's disease striatum. Human brain protein extracts were obtained from Huntington's disease (HD), Amyotrophyc Lateral Sclerosis (ALS) and Parkinson's disease (PD) patients from Striatum Immunoblots were performed with antibody against HSF1 and GAPDH as loading control. Tissue was evaluated for three control (C) individuals and three disease patients for each disease (HD, ALS, PD) and individuals were sex-matched (M, male; F, female) and closely age-matched.

FIG. 10 shows chemical inhibitors of Casein Kinase II (CK2) rescue HSF1 activation, Hsp70 expression levels, decrease Htt-Q74 protein aggregation and enhance cell viability in Htt-Q74 expressing cells. Htt-Q74 expressing P12 cells were incubated at 37° C. in the presence of either DMSO or the casein kinase (CK2) inhibitors (A) SB216763 (B) TID43 or (C) Emodin at 0.5, 1 and 5 μM final concentration for 15 h prior to Htt-Q74 protein induction by doxycycline. Then cells were either not treated (−Dox) or treated with doxycycline at 1 microgram/mL during 3 days (+Dox) at 37° C. Cells were then heat shocked at 42° C. (1 h) followed by 7 h recovery (HS) at 37° C. Total protein was analyzed by SDS-PAGE and immunoblotting for Hsp70 and GAPDH. (D) Cells from CK2 kinase inhibitor treatment were analyzed by fluorescence microscopy using GFP as a marker for Htt-Q74 aggregation and the number of cells with inclusions bodies was quantified and normalized with total cell number from DIC images, as a measure of Htt-Q74 protein aggregation. Data are presented as mean±SE from three separate experiments (* P<0.05, ** P<0.01). (E) Htt-Q74 PC12 cells were incubated at 37° C. in a 96-well plate in the presence of either DMSO or the kinase inhibitor TID43 at 5 microMolar final concentration during 15 h prior to Htt-Q74 induction by doxycycline. Cells were then either not treated or treated with doxycycline at 1 microgram/mL for 3 days (+Dox) at 37° C. and then heat shocked at 42° C. (1 h) followed by 7 h recovery (HS) at 37° C. Cell viability was analyzed using the Cell Titer Glo assay (Promega) and values were expressed as percentage of viable cells compared with untreated cells at 37° C. Data are presented as means±SE from three separate experiments (* P<0.05).

FIG. 11 shows CK2 kinase inhibitor increases HSF1 protein levels and activation of Hsp70 gene expression, while decreasing HSF1 phospo-Ser303 levels in Htt-Q74 expressing cells. Htt-Q74 PC12 cells were either not treated (−Dox) or treated with doxycycline at 1 microgram/mL during 3 days (+Dox) at 37° C. After 24 h and 48 h of dox induction cells were supplemented with 0.5 or 1 micromolar TID43. After 72 h cells were then heat shocked at 42° C. (1 h) followed by 7 h recovery (HS) at 37° C. Total protein was analyzed by SDS-PAGE and immunoblotting for Hsp70, HSF1, HSF1-phosphoSer303 and GAPDH. Results shown are from the same blot and are separated by vertical lines where two regions of the same blot were joined to focus on specific data.

FIGS. 12, 13 and 14 show CK2 inhibiting agents obtained from Dowling, et al., Med. Chem Letters 2012 3:278-283.

FIGS. 15-23 show CK2 inhibiting agents obtained from Bortolato, Andrea 31 Jan. 2008 Universita Degli Studi Di Padova—Doctoral Thesis “Rational Design of New Protein Kinases Inhibitors of Pharmaceutical Interest.

FIG. 24 shows CK2 inhibiting agents obtained from EP1911451.

FIG. 25 shows CK2 inhibiting agents obtained from Pagano, et al., 2008 Biochem. J. 415:353-365.

FIGS. 26 and 27 show CK2 inhibiting agents obtained from Janeczko, et al., 2012 European J. Med. Chem. 47:345-350.

FIGS. 28-34 show CD2 inhibiting agents obtained from Cozza, et al., 2013 Current Med. Chem. 20:671-693.

FIGS. 35-45 show CD2 inhibiting agents obtained from Cozza, et al., 2012 Expert Opin. Ther. Patents 22(9):1081-1097.

FIG. 46 shows that both HSF1 and Hsp70 levels are progressively lower in muscle of a Huntington's Disease mouse model compared to a Wild Type control mouse.

FIG. 47 shows reduction in CK2 catalytic subunit expression by RNAi elevates HSF1 protein levels and enhances Hsp70 gene activation in Htt-Q74 expressing cells. PC12 cells were transiently transfected with siRNA against different CK2 alpha subunits α1 (a) or α2 (α′) or both together α1α2 (α α′). Two days after RNAi transfection the cells were either not treated (−Dox) or treated with doxycycline at 1 microgram/mL for 2 days (+Dox) at 37° C. to induce Htt-Q74 protein expression. Cells were then heat shocked at 42° C. (1 h) followed by 7 h recovery (HS) at 37° C. Total protein was analyzed by SDS-PAGE and immunoblotting for CK2, Hsp70, HSF1, HSF1-phospho-S303 and GAPDH.

FIG. 48 shows that decreased HSF1 and Hsp70 levels in striatal tissue is reflected by decreases in HSF1 and Hsp70 in skeletal muscle in the zQKI175 HD mouse model.

FIG. 49 shows increased CK2α′ in cellular/mouse HD models and HD patient striatum. (A) PC12-HttQ74 cells were cultivated at 37° C. (C) in the presence of Dox for 3 days, exposed to heat shock 1 h at 42° C., followed by recovery period at 37° C. for 7 h (HS). Extracts were immunobloted for CK2 subunits. (B) Striatal protein samples from Wild type and KIQ175 mice at 6 or 12 months immunoblotted for CK2 subunits. (C) Striatal extracts from 2 HD patients and 2 age and sex-matched controls from the Harvard BioBank analyzed by immunoblotting for CK2 subunits. GAPDH was used as loading control.

FIG. 50 shows CK2α′ knock out mice show elevated HSF1 and Hsp70 in mouse striatum. Two independent wild type (WT) or CK2α′ knock out (CK2α′^{-/-}) mice were evaluated by immunoblotting of striatal tissue for CK2α′, HSF1, Hsp70 and GAPDH as loading control.

FIG. 51 shows heterozygous loss of CK2α′ in KI175 HD mice enhances thalamostriatal excitatory synapse formation. Mice of the indicated genotype (4/cohort) were analyzed for corticostriatal (VGlut1) and thalamostriatal (VGlut2) excitatory synapse number in the Dorsal striatum at 5 weeks of age.

FIG. 52 shows CK2α′ reduction increases striatal HSF1, Hsp70 protein levels in KI175 mouse. WT or KIQ175 (HD) mice were crossed to generate mice of the indicated genotype with respect to CK2α′ and HD. Striatal protein extracts from 6 month mice were blotted for CK2α′, CK2α, CK2b, HSF1, Hsp70 and GAPDH. Note the increase in CK2α′ and decrease in HSF1 and Hsp70 in the HD mouse compared to the WT mouse (compare lane 1 and 2). Note also the increase in HSF1 and Hsp70 in the CK2α′+/−HD mouse, compared to the HD mouse (compare lanes 1 and 3).

FIG. 53 shows CK2α′ heterozygous state in the KI175 mouse Huntington's Disease model ameliorates weight loss. Body weight of KI175 HD mouse model, WT mice, KIQ175/CK2α′(+/−) mice and CK2α′(+/−) mice at 6 months of age.

FIG. 54 shows additional compounds identified as CK2 inhibitors.

### DETAILED DESCRIPTION OF THE INVENTION

The proper synthesis, folding, trafficking, modifications, interactions, biochemical activities and eventual clearance of cellular proteins is essential for normal growth, development and maintenance during the life cycle of all organisms. Inappropriate folding, aggregation and accumulation of abnormal proteins is proteo-toxic to cells due to their dominant effects of insolubility, inappropriate interactions and long half-lives (see, e.g., Johnson, J, et. al., Cell. 1997 90(2), 201-204; Bukau, B, et. al., Cell. 2000 101(2), 119-122; Hartl, F, et. al., (1996) Nature. 1996 381, 571-580; Deuerling, E, et. al., Crit. Rev. Biochem. Mol. Bio. 2004 39, 261-277; Bukau, B., et. al., Cell 2006 125(3), 443-451; and Dickey, C, et. al., Trends in Mol. Med. 2007 13(1), 32-38).

Neuronal tissues and cells are exquisitely sensitive to defects in protein folding, aggregation and clearance and these defects are causally or correlatively associated with diseases that include Huntington's disease, Parkinson's disease, Alzheimer's disease, Amyotropic Lateral Sclerosis, prion diseases and other neurodegenerative disorders (see, e.g., Bonini, N. Proc. Natl. Acad. Sci., USA, 2002 99, 16407-16411; Muchowski, P, Neuron 2002 35, 9-12; Morimoto, R, New England J. Med. 2006 355, 2254-2255; Finkbeiner, S, et. al., J. Neurosci. 2006 26(41), 10349-10357; Furukawa, Y., et. al., PNAS. 2006 103(18), 7148-7153; Gidalevitz, T, et. al., Science 2006 311, 1471-1474). Many of these are diseases occur frequently in the elderly and result in a variety of symptoms due to loss of function of motor, dopaminergic and other neurons essential for a normal healthy life (see, e.g., Cummings, C. J. and Zoghbi, H, Hum. Mol. Genet. 2000 9, 909-916). Defects in protein folding, aggregation and clearance have also been implicated in type 2 diabetes mellitus (see, e.g., Chung, J, et. al. PNAS 2008 105(5), 1739-1744). Several lines of evidence suggest that a familiar form of amyotrophic lateral sclerosis (ALS) is associated with the mis-folding and aggregation of mutated Cu/Zn superoxide dismutase (SOD1), one of the most abundant proteins in motor neurons (see, e.g. Prudencio, M, et. al., Human Molecular Genetics. 2009 Sep. 1, 18(17), 3217-26.). A second protein, ataxin-2 (Elden, et. al. Nature 2010 466, 1069-1075) showed that ataxin-2 intermediate-length polyglutamine expansions are associated with increased risk for ALS. Elevated levels of cellular proteins that carry out protein folding, protein chaperones, protect motor neurons from the toxic effects of misfolded SOD1. Given the great potential for a therapeutic role of elevated protein chaperone levels in ALS, small molecule elevation of the natural protein folding machinery in motor neurons is a promising avenue for the treatment of ALS.

Aging is associated with enhanced protein aggregation and generation of protein inclusions in virtually all cell types. Interestingly, several age-related neurodegenerative diseases like amyotrophic lateral sclerosis or Parkinson disease are directly associated with protein aggregation in distinct regions of the central nervous system despite the ubiquitous expression of affected proteins. Modification of the chaperone network can be beneficial for disease progression. See Kern, et al., “2010 HSF1-Controlled and Age-Associated Chaperone Capacity in Neurons and Muscle Cells of C. elegans.” PLoS ONE 5.

While many aspects of these complex processes of protein misfolding and aggregation are incompletely understood, a variety of individual protein chaperones and co-chaperone complexes function to fold, process, mature and degrade cellular proteins. (see, e.g., Johnson, J and Craig, E, 1997) Cell. 1997 90(2), 201-204; Bukau, B, et. al., Cell. 2000 101(2), 119-122; Hartl, F, Nature 1996 381, 571-580; Deuerling, E., and Bukau, B. (2004) Crit. Rev. Biochem. Mol. Bio. 2000 39, 261-277; Bukau, B., et. al., Cell. 2006 125(3) 443-451; Dickey, C, et. al., Trends in Mol. Med. 2007 13(1), 32-38). Many neurodegenerative diseases are caused, for example, by genetically programmed changes in specific proteins, such as through the addition of poly glutamine (polyQ) coding sequences, by genetic defects in the protein folding and processing machinery, or by as yet poorly understood mechanisms by which abnormal protein conformations can be propagated in a protein-catalyzed fashion (see, e.g., Bonini, N, Proc. Natl. Acad. Sci., USA. 2002 99, 16407-16411; Muchowski, P, (2002) Neuron. 2002 35 9-12; Morimoto, R New England J. Med. 2006 355, 2254-2255; Finkbeiner, S, et. al., (2006) J. Neurosci. 2006 26(41) 10349-10357; Furukawa, Y, et. al., PNAS 2006103(18), 7148-7153; Gidalevitz, T, et. al., Science 2006 311, 1471-1474; Cummings, C and Zoghbi, H Hum. Mol. Genet. 2000 9, 909-916).

Protein chaperones facilitate the folding, stabilization, solubilization and degradation of cellular proteins and are often included in the group of Heat Shock Proteins (Hsps) because their synthesis is elevated in response to heat and other stresses known to induce protein unfolding, aggregation and degradation (see, e.g., Morimoto, R. I., Tissieres, A., and Georgopoulos, C. (1994) The Biology of Heat Shock Proteins and Molecular Chaperones, Cold Springs Harbor Laboratory Press, Cold Springs Harbor N.Y. 1994; Lindquist, S. (1992) Curr. Opinion in Genet. and Develop. 1992 2, 748-755; Feige, U, et. al., (eds.) Stress-inducible cellular responses. Vol 77, Birkhauser, Verlag, Boston; Lindquist, S and Craig, E Ann. Rev. Genet. 1988 22, 631-677).

Protein chaperones act independently and in concert to ameliorate biochemical hallmarks or symptoms of the disease associated with unfolded or aggregated proteins. For example, in mammalian cell culture, mouse or Drosophila models of polyQ aggregation or alpha-synuclein toxicity, expression of the Hsp70 or Hsp40 chaperones can significantly suppress protein aggregation, increase protein solubility and turnover and ameliorate neuronal loss (see, e.g., Bailey, C, et. al., Hum. Mol. Genet. 2002 11(5), 515-523; Kitamura, A, et. al., Nat. Cell Bio. 2006 8(10), 1163-1170; Pavan, K., et. al., Science. 2002 295, 865-868; Chai, Y, et. al., J. Neurosci. 1999 19(23), 10338-10347; Muchowski, P, et. al., Proc. Natl. Acad. Sci. USA. 2000 97, 7841-7846; Jana, N, et. al., Hum. Mol. Genet. 2000 9, 2009-2018; Wyttenbach, A, et. al., (Proc. Natl. Acad. Sci. 2000 97, 2898-2903; Adachi, H, et. al., J. Neurosci. 2003 23, 2203-2211; Cummings, C, et. al., Hum. Mol. Genet. 2001 10, 1511-1518).

Additional studies suggest that Hsp70 and Hsp40 can synergize in the suppression of polyQ-mediated neuronal degeneration and that arimoclomal, an inducer of Hsp synthesis, significantly delays disease progression in a mouse model of ALS (see, e.g., Kieran, D, et. al., Nature Medicine. 200410, 402-405). From bacteria to human cells, Hsp synthesis is coordinately induced in response to stress conditions that result in protein unfolding, aggregation and proteolysis by stress-responsive transcription factors.

In cells from yeast to humans, the transcription of genes encoding Hsps is induced in response to stresses such as increased temperature through cis-acting promoter elements called Heat Shock Elements (HSEs), composed of variations of the inverted repeated pentameric consensus sequence 5″-nGAAnnTTCnnGAAn-3′ (SEQ ID NO: 1) (see, e.g., Morimoto, R et al., The Biology of Heat Shock Proteins and Molecular Chaperones, Cold Springs Harbor Laboratory Press, Cold Springs Harbor N.Y. 1994; Lindquist, S and Craig, E Ann. Rev. Genet. 1988 22, 631-677). In response to stress the Heat Shock Transcription Factor, HSF, binds as a homo-trimer to HSEs and activates target gene transcription. Indeed, HSFs and their cognate DNA binding site HSEs are two highly structurally and functionally conserved cis- and trans-acting regulatory factors. The baker's yeast Saccharomyces cerevisiae harbors a single gene encoding HSF that is essential for cell viability under all conditions tested. Recent genome-wide expression and chromatin-immunoprecipitation experiments demonstrate that yeast HSF directly activates a broad range of genes encoding proteins that function as chaperones, in protein turnover and a variety of additional stress protection roles (see, e.g., Hahn, J, et. al., Molecular and Cellular Biology 2004 24, 5249-5256).

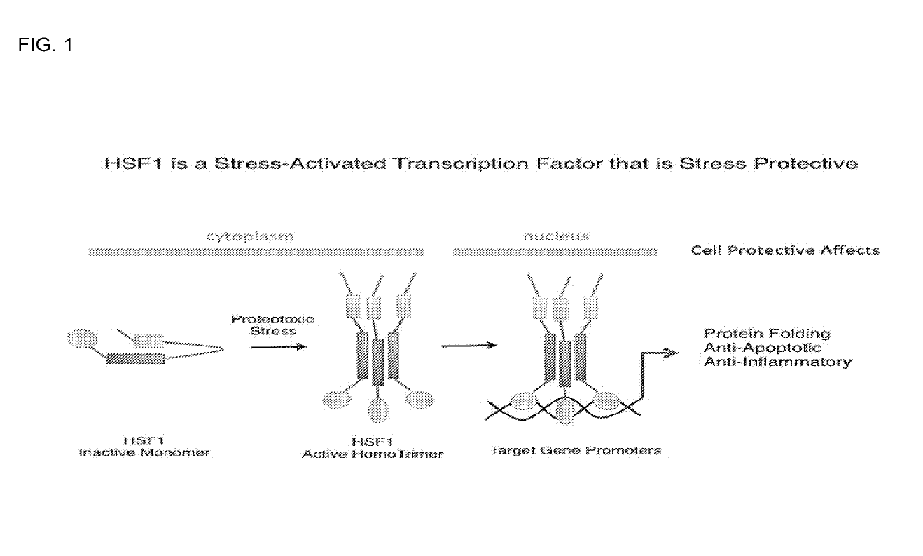

In mammals, Drosophila and C. elegans HSF1 responds to stress to activate transcription of genes encoding a family of protein chaperones (Wu, C Ann. Rev. Cell Dev. Biol. 1995 11, 441-469; Pirkkala, L., Nykanen, et. al., FASEB J. 200115, 1118-1131; Hsu, A. L., (2003) Science 300: 1142-1145; Morley, J. F., and Morimoto, R. I. (2004) Mol. Bio. Cell 15: 657-664). While the precise mechanisms whereby HSF1 from humans and other organisms sense and respond to stress have not been elucidated, a model that summarizes current understanding of this process in human cells is shown in FIG. 1. HSF1 activation is a multi-step process that occurs post-translationally in response to elevated temperatures, the accumulation of unfolded proteins and other stressful conditions.

In the absence of acute stress, HSF1 is present largely in the cytoplasm as a monomer, and is thought to be associated with Hsp90, Hsp70 and other proteins (see, e.g., Zuo, et. al., Cell. 1998 94, 471-480; Ali, A, et. al., (1998) Mol. Cell. Biol. 199818, 4949-4960; Guo, Y., et. al., J. Biol. Chem. 2001 276, 45791-45799). In vitro and in vivo experiments suggest that HSF1 is retained in the momomeric state through intramolecular interactions between two coiled-coil regions, Leucine Zipper 1-3 (LZ1-3) and Leucine Zipper 4 (LZ4) (see, e.g., Rabindran, S, et. al., Science. 1993 259, 230-234. Indeed, point mutations in Leucine Zipper 4 (HSF1lz4m) cause constitutive HSF1 homo-trimerization in mammalian cells. In response to stress, HSF1 is converted to a homo-trimer that is stabilized by inter-molecular coiled-coil interactions and accumulates in the nucleus, where it engages in high affinity binding to HSEs within target gene promoters and activates target gene transcription. Hsp target gene activation by HSF1 is transient, and correspondingly, HSF1 is ultimately converted back to the low affinity DNA binding monomeric form in the cytosol. HSF1 is phosphorylated both under basal conditions where this modification is thought to maintain the protein in an inactive state and in response to stress, with this latter modification having functional consequences that are not well understood.

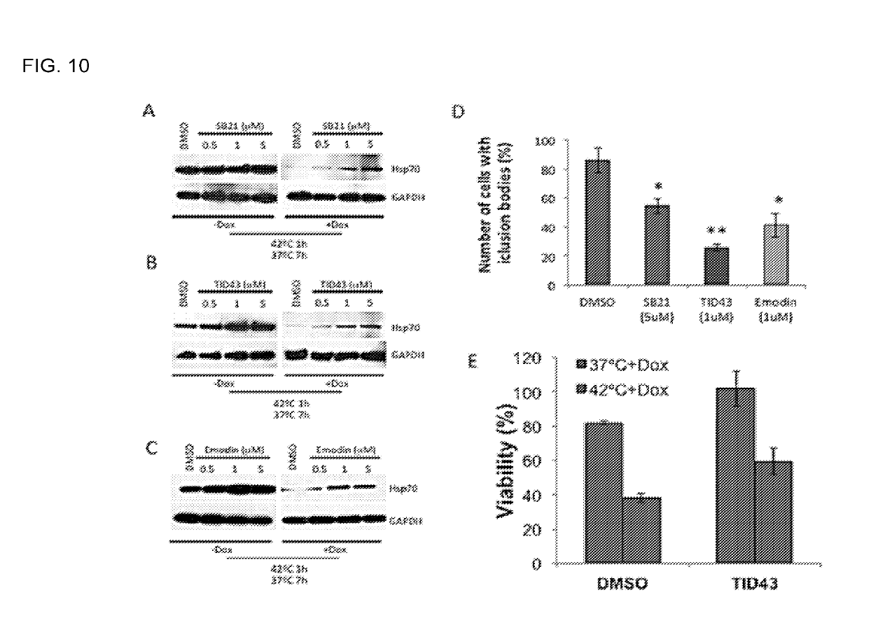

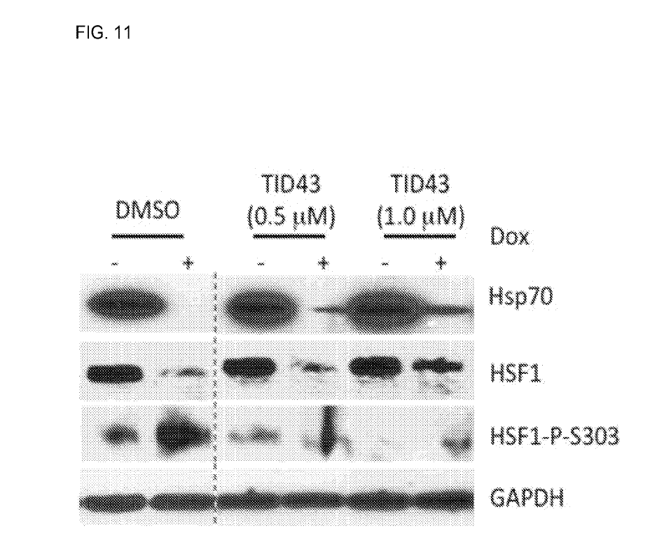

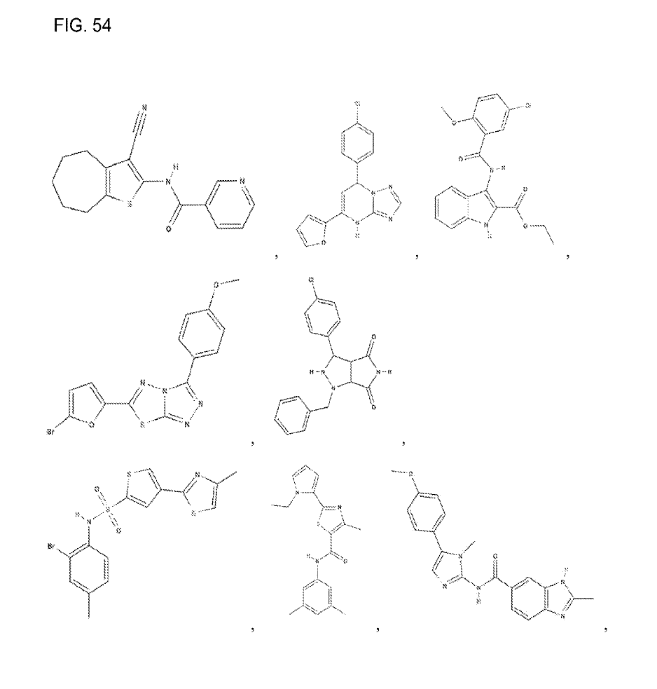

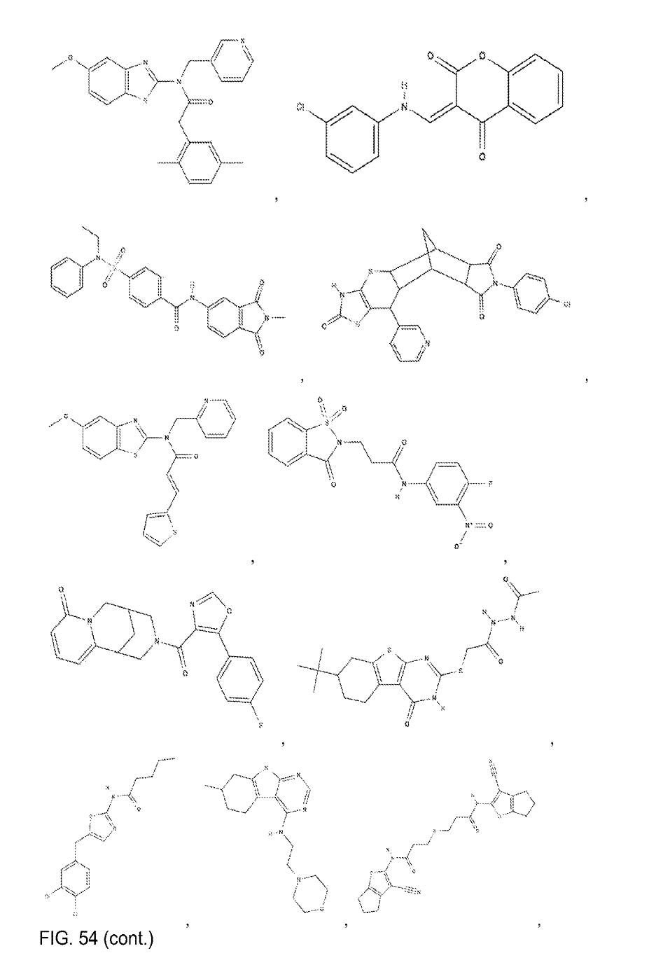

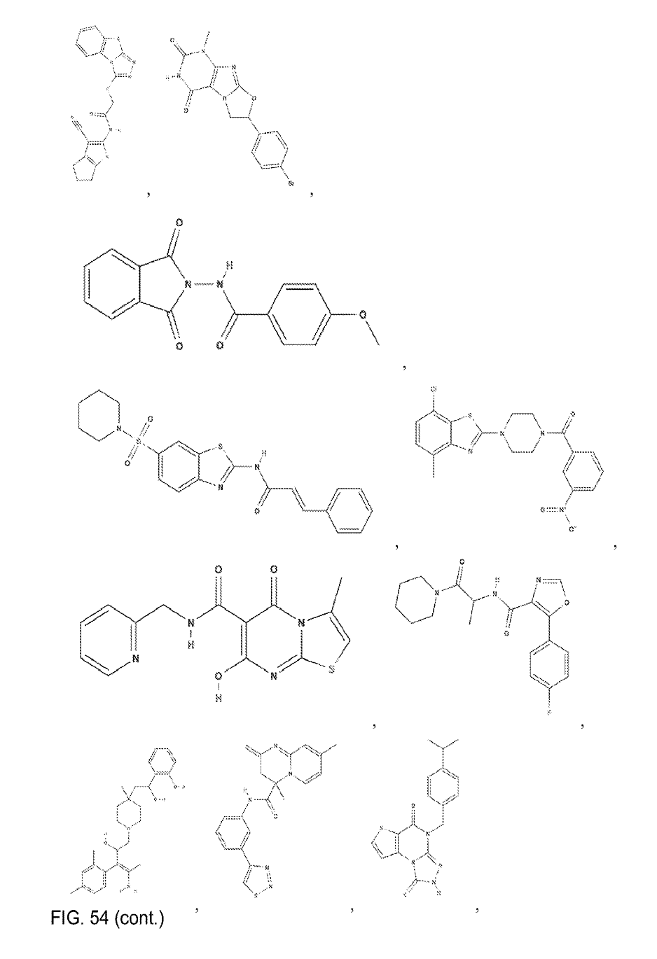

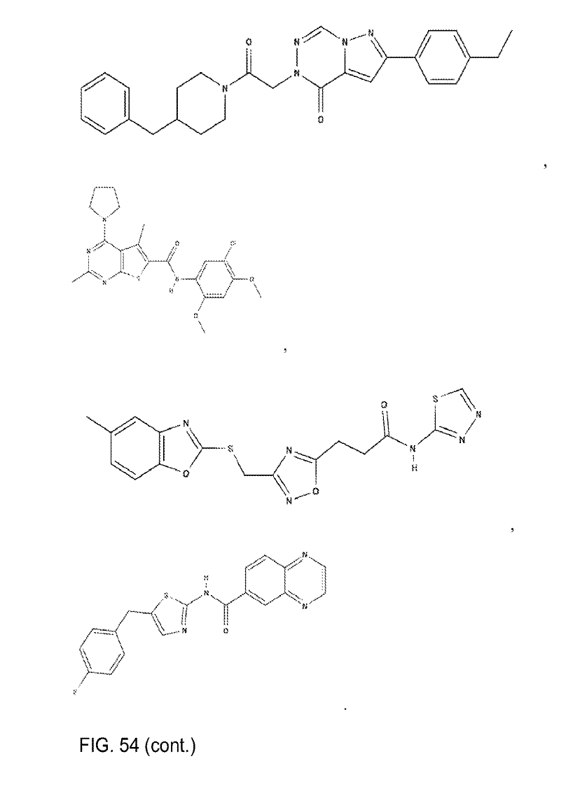

Experiments conducted during the course of developing embodiments for the present invention discovered that the use of Casein Kinase 2 (CK2) inhibitors restores HSF1 protein levels, protein chaperone expression levels, reduces poly-glutamine protein aggregation and enhances cell viability in cells expressing a pathologic Huntington's Disease protein (Htt-Q74) (FIGS. 10 and 11). Indeed, chemical inhibitors of Casein Kinase 2 (CK2) were shown to rescue cellular HSF1 protein levels and their activation, Hsp70 expression levels, decrease Htt-Q74 protein aggregation, and enhance cell viability in Htt-Q74 expressing cells. Moreover, CK2 kinase inhibitors were shown to increase HSF1 protein levels and activation of Hsp70 gene expression, while decreasing HSF1 Ser303 and/or Ser307 phosphorylation levels in Htt-Q74 expressing cells. In addition, it was shown that HSF1 protein levels, and HSF1 target protein levels such as Hsp70, are progressively lower in muscle of a Huntington's Disease mouse model (see FIG. 46). Moreover, experiments were conducted showing CK2 inhibiting activity for specific compounds (see, Example 8 and FIG. 54).

Accordingly, the present invention provides compositions and methods capable of increasing the protein levels of and activating Heat Shock Factors (e.g., facilitating HSF1 homo-trimerization), activating heat shock factor (e.g., HSF1) target gene expression (e.g., Heat Shock Elements) and protein expression (e.g., Heat Shock Proteins), therapeutic and/or research uses, and methods for identifying subjects suffering from conditions involving aberrant HSF1 protein levels and/or activation. In addition, the present invention contemplates that CK2 inhibitors have therapeutic benefit by increasing HSF1 protein levels and/or activity beyond normal physiological levels. Moreover, the present invention contemplates that CK2 inhibitors have therapeutic benefit by increasing HSF1 protein levels and/or activity when HSF1 protein levels or activity are lower than normal.

As noted, experiments conducted during the course of development of embodiments for the present invention determined that inhibition of CK2 activity through application of CK2 inhibiting agents restored HSF1 protein levels, restored protein chaperone expression levels, and reduced poly-glutamine protein aggregation and enhances cell viability in cells expressing a pathologic Huntington's Disease protein (Htt-Q74).

CK2 is a hetero-tetrameric ubiquitous kinase consisting of two catalytic subunits and two regulatory subunits. The two catalytic subunits, α and α′, are highly homologous but differ in their C-terminal regions. It is not known whether CK2α and α′ have distinctive substrate specificity. Thus, it is assumed that the two kinase isoforms at least partially overlap in their substrate specificity. CK2 protein levels and activity were found to be elevated in the brain when compared to other organs. Here we have studied the protein levels of CK2α and α′ isoforms in major brain regions. CK2α and α′, are expressed in all brain regions tested. Whereas CK2α levels do not vary strongly across the regions, CK2α′ levels are slightly higher in the cortex and hippocampus than in other regions. Furthermore, CK2α protein levels in the striatum are relatively high when compared to CK2α′. The approximate stoichiometry ratio of CK2α:CK2α′ is 8:1. Therefore, one can consider that CK2α levels are predominant in comparison to CK2α′ levels throughout the mammalian brain (see, e.g., Ceglia, et al., 2011 Mol. Cell Biochem. 356:169-175).

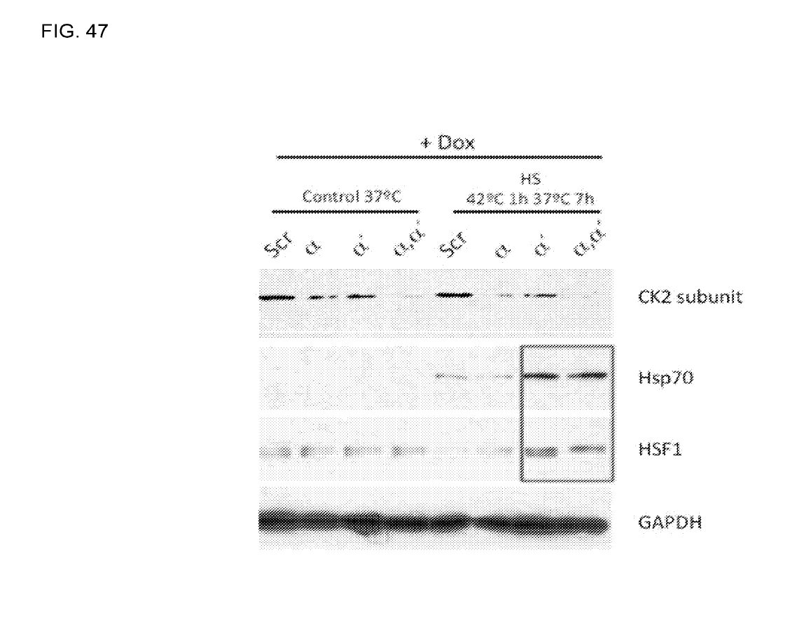

Experiments conducted during the course of developing embodiments for the present invention further demonstrated that CK2 inhibitors that specifically target the CK2 alpha/alpha prime catalytic subunits rescue HSF1 levels in polyQ protein expressing cells. Indeed, when alpha and alpha prime were tested separately, alpha and alpha prime were the key target to inhibit for HSF1 protein stabilization and to elevate HSF1 protein levels. In fact, specific CK2 inhibitors using silencing RNA for alpha prime inhibition were specific for therapeutic purposes (FIG. 47).

As such, the present invention provides CK2 inhibiting agents for purposes of facilitating HSF1 homo-trimerization, activating HSF1 target gene expression (e.g., Heat Shock Elements), elevating HSF1 protein expression levels, activating protein chaperone expression and activity (e.g., increased protein folding, increased protein solubilization, protein degradation). An understanding of the mechanism by which such CK2 inhibiting agents activate HSF proteins is not required to practice the present invention.

As used herein, a “CK2 inhibitor” is a compound that interferes with casein kinase 2 activity. CK2 inhibitors are well known to those of ordinary skill in the art.

The present invention is not limited to use of specific CK2 inhibiting agents. In some embodiments, the CK2 inhibiting agents are shown in FIG. 54 and described in Example 8. In some embodiments, the CK2 inhibiting agent is SB216763 (via inhibition of GSK3). In some embodiments, the CK2 inhibiting agent is TID43. In some embodiments, the CK2 inhibiting agent is Emodin. Additional examples of applicable CK2 inhibiting agents include, but are not limited to, TBBz, 7,8-dichloro-1,4-dihydro-4-oxo-3-quinolinecarboxylic acid (Cay10577), resorufin (Sandholt, et al., 2009 Anticancer Drugs 20(4):238-248), TBCA, Quinalizarin and its SAR (Giorgio, et al., 2009 Biochem. J. 421:387-395), AZ285, TID43, TBB and other related molecules, CX-4945 and related molecules, CX-5011, CX-5279, TBI, TBCA, DMAT, CIBG-300, Ellagic Acid, K64/PBIN, K66/TMCB, IQA, Fisetin, Hematein, FLC21, TID46, Quinolone 9, Quinolone 7, TTP22, FNH79.

In some embodiments, applicable CK2 inhibiting agents include 7-Amino-5-(4-methoxyphenyl)pyrazolo[1,5-a]pyrimidine-3-carbonitrile, 7-(Cyclopropylamino)-5-(2-thienyl)pyrazolo[1,5-a]pyrimidine-3-carbonitrile, 7-(Cyclopropylamino)-5-(4-methoxyphenyl)pyrazolo[1,5-a]pyrimidine-3-carbonitrile, 5-(4-Methoxyphenyl)-7-[(1-methyl-1H-pyrazol-3-yl)amino]pyrazolo[1,5-a]pyrimidine-3-carbonitrile, 7-(Cyclopropylamino)-5-(6-methoxypyridin-3-yl)pyrazolo[1,5-a]pyrimidine-3-carbonitrile, N-(1-(3-cyano-7-(cyclopropylamino)pyrazolo[1,5-a]pyrimidin-5-yl)-1H-indol-6-yl)acetamide, N-(1-(3-cyano-7-(cyclopropylamino)pyrazolo[1,5-a]pyrimidin-5-yl)-1H-indazol-6-yl)acetamide, N-[1-[3-cyano-7-(cyclopropylamino)pyrazolo[1,5-a]pyrimidin-5-yl]pyrrolo[3,2-b]pyridin-6-yl]acetamide, N-[1-[3-cyano-7-(cyclopropylamino)pyrazolo[1,5-a]pyrimidin-5-yl]indolin-6-yl]acetamide, N-{4-[3-cyano-7-(cyclopropylamino)pyrazolo[1,5-a]pyrimidin-5-yl]-3,4-dihydro-2H-1,4-benzoxazin-6-yl}acetamide, N-(1-(3-cyano-7-(cyclopropylamino)pyrazolo[1,5-a]pyrimidin-5-yl)-1,2,3,4-tetrahydroquinolin-7-yl)acetamide, N-(1-(3-cyano-7-(cyclopropylamino)pyrazolo[1,5-a]pyrimidin-5-yl)-5-methyl-1H-indol-6-yl)acetamide, N-(1-(3-cyano-7-(1-methyl-1H-pyrazol-3-ylamino)pyrazolo[1,5-a]pyrimidin-5-yl)-1H-indol-6-yl)acetamide, N-(1-(3-cyano-7-(oxetan-3-ylamino)pyrazolo[1,5-a]pyrimidin-5-yl)-1H-indol-6-yl)acetamide, N-(1-(3-cyano-7-(4-hydroxybutylamino)pyrazolo[1,5-a]pyrimidin-5-yl)-1H-indol-6-yl)acetamide, N-(1-(3-cyano-7-(2-morpholinoethylamino)pyrazolo[1,5-a]pyrimidin-5-yl)-1H-indol-6-yl)acetamide, N-(1-(3-cyano-7-(2-(pyrrolidin-1-yl)ethylamino)pyrazolo[1,5-a]pyrimidin-5-yl)-1H-indol-6-yl)acetamide, N-(1-(3-Cyano-7-(3-(dimethylamino)propylamino)pyrazolo[1,5-a]pyrimidin-5-yl)-1H-indol-6-yl)acetamide, N-(1-(3-Cyano-7-(3-(pyrrolidin-1-yl)propylamino)pyrazolo[1,5-a]pyrimidin-5-yl)-1H-indol-6-yl)acetamide, N-(1-(3-Cyano-7-(cyclopropylamino)pyrazolo[1,5-a]pyrimidin-5-yl)-1H-indol-6-yl)-N-methylacetamide, 7-(Cyclopropylamino)-5-(6-(hydroxymethyl)-1H-indol-1-yl)pyrazolo[1,5-a]pyrimidine-3-carbonitrile, 7-(cyclopropylamino)-5-(6-(methylsulfonylmethyl)-1H-indol-1-yl)pyrazolo[1,5-a]pyrimidine-3-carbonitrile, N-{3-[3-cyano-7-(cyclopropylamino)pyrazolo[1,5-a]pyrimidin-5-yl]-1-methyl-1H-indol-5-yl}acetamide, N-(3-(3-cyano-7-(cyclopropylamino)pyrazolo[1,5-a]pyrimidin-5-yl)-1H-indol-5-yl)acetamide, N-(3-(3-cyano-7-(cyclopropylamino)pyrazolo[1,5-a]pyrimidin-5-yl)-1-(2-hydroxyethyl)-1H-indol-5-yl)acetamide, N-(3-(3-cyano-7-(cyclopropylamino)pyrazolo[1,5-a]pyrimidin-5-yl)-1-(3-hydroxypropyl)-1H-indol-5-yl)acetamide, Methyl 3-(5-acetamido-3-(3-cyano-7-(cyclopropylamino)pyrazolo[1,5-a]pyrimidin-5-yl)-1H-indol-1-yl)propanoate, 3-(5-Acetamido-3-(3-cyano-7-(cyclopropylamino)pyrazolo[1,5-a]pyrimidin-5-yl)-1H-indol-1-yl)propanoic acid, 7-(cyclopropylamino)-5-(6-nitro-1H-indazol-1-yl)pyrazolo[1,5-a]pyrimidine-3-carbonitrile, 5-(6-Amino-1H-indazol-1-yl)-7-(cyclopropylamino)pyrazolo[1,5-a]pyrimidine-3-carbonitrile (Dowling, et al., 2012 Med. Chem. Lett. 3:278-283).

In some embodiments, applicable CK2 inhibiting agents include CX-4945, CX-5011, CX-5279, and CX-5033 (Kang, et al., 2009 PLoS One 4(8):e6611), 5-oxo-5,6-dihydroindolo-(1,2-a)quinazolin-7-yl]acetic acid (IQA), and 4,5,6,7-tetrabromobenzotriazole (TBB).

In some embodiments, applicable CK2 inhibiting agents include CX-8184 (Drygin, et al., 2012 Cancer Res. 72(8)Supp1), and 5-oxo-5,6-dihydroindolo[1,2-a]quinazolin-7-yl)acetic acid (Vangrevelinghe, et al., 2003 J. Med. Chem. 46(13):2656-2662).

In some embodiments, applicable CK2 inhibiting agents include myricetin, quercetin, fisetin, kaempferol, luteolin, and apigenin (Lolli, et al., 2012 Biochem. 51(31):6097-6107). In some embodiments, the CK2 inhibiting agents are provided within pharmaceutical compositions.

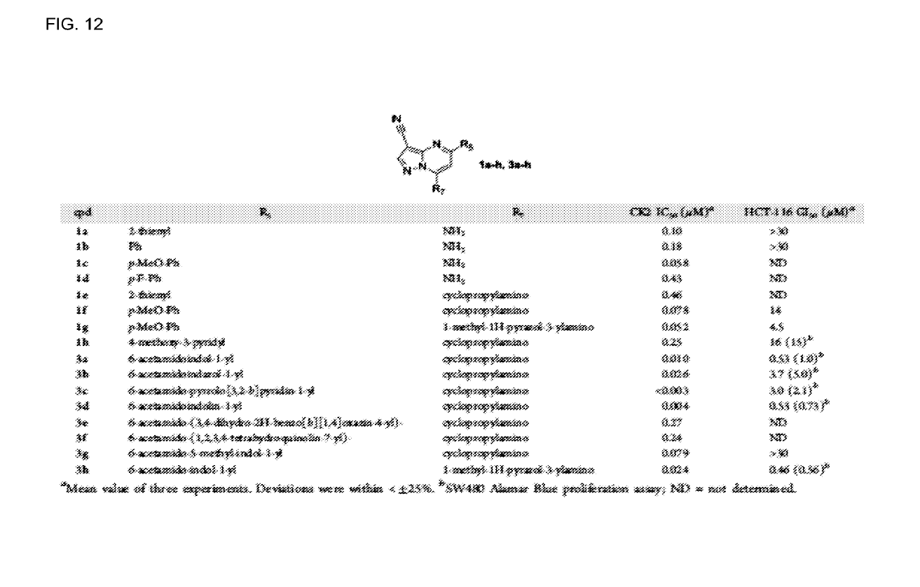

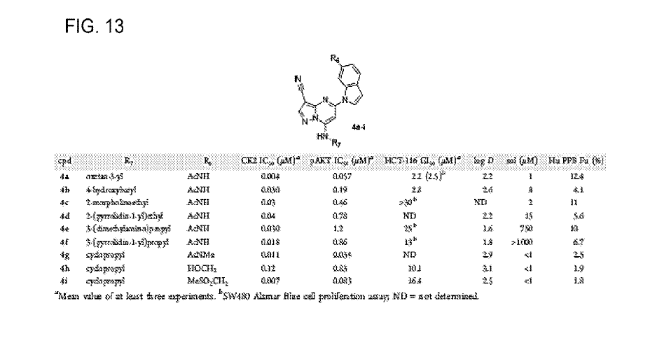

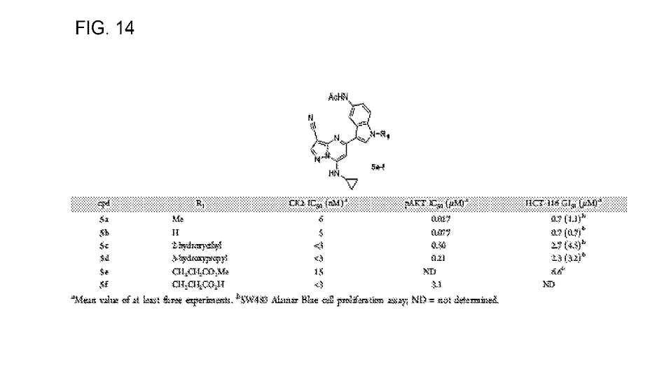

In some embodiments, applicable CK2 inhibiting agents include any of the compounds shown in FIGS. 12, 13 and 14 (compounds shown in Dowling, et al., Med. Chem Letters 2012 3:278-283).

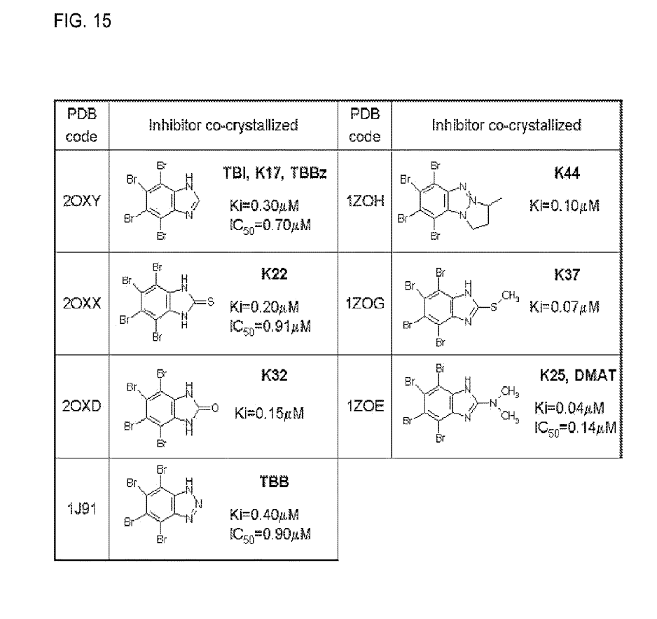

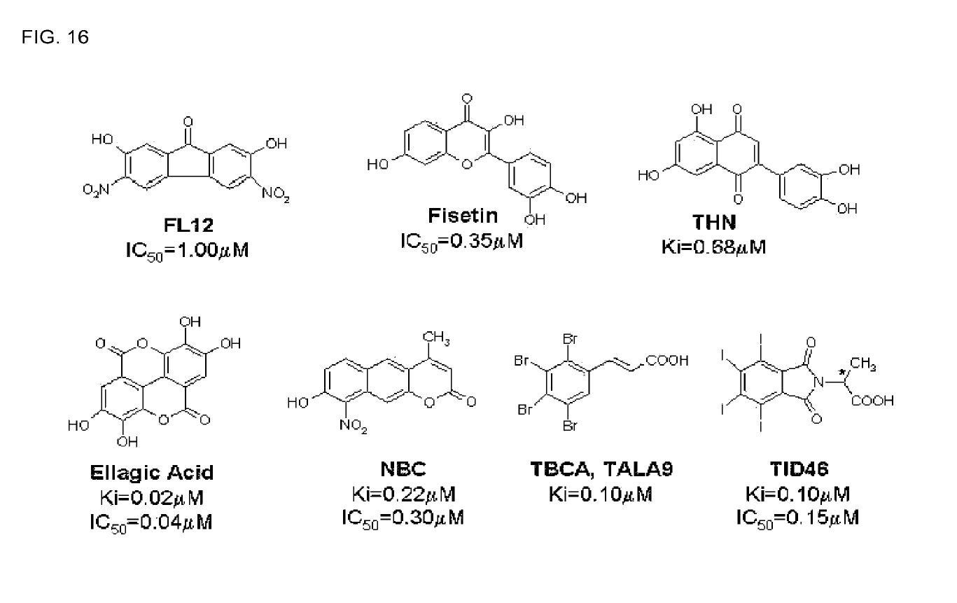

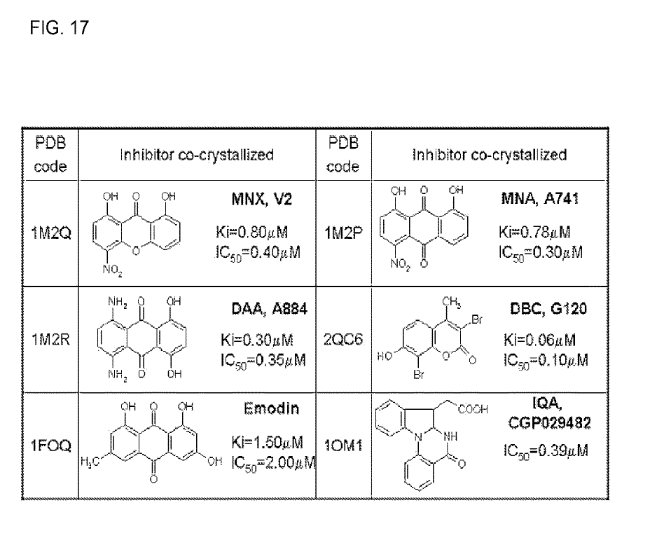

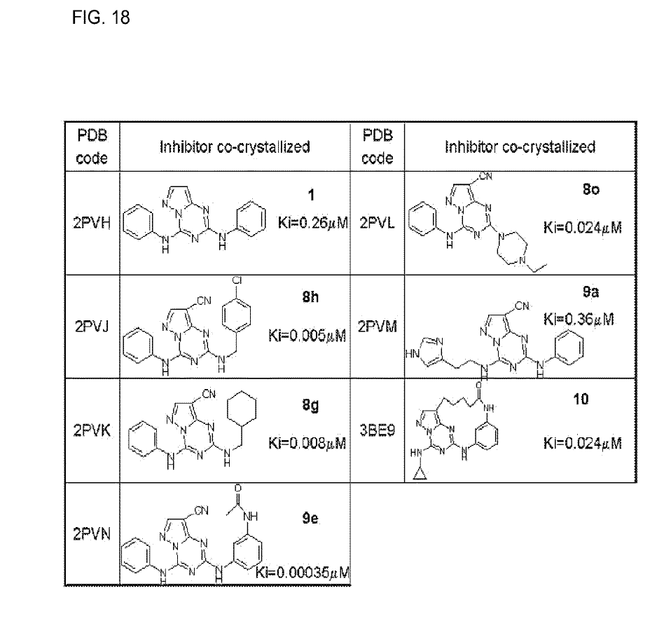

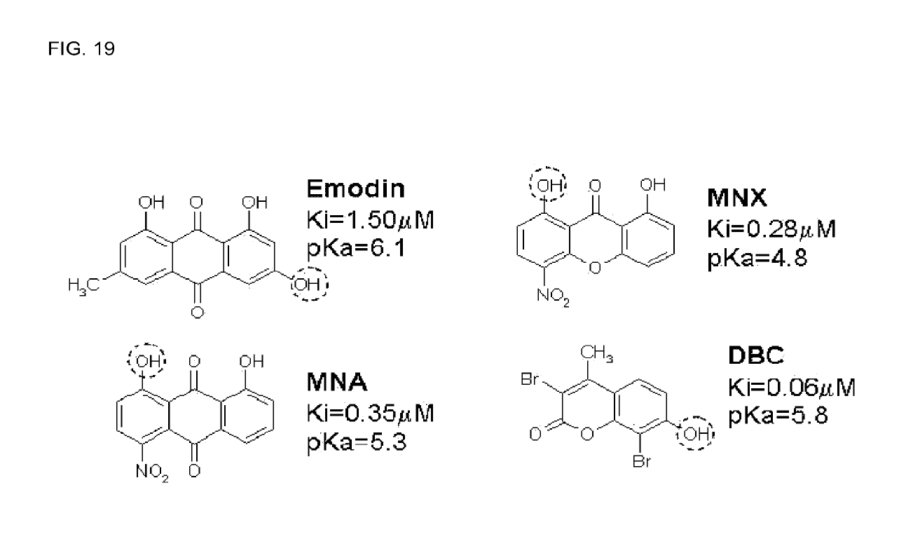

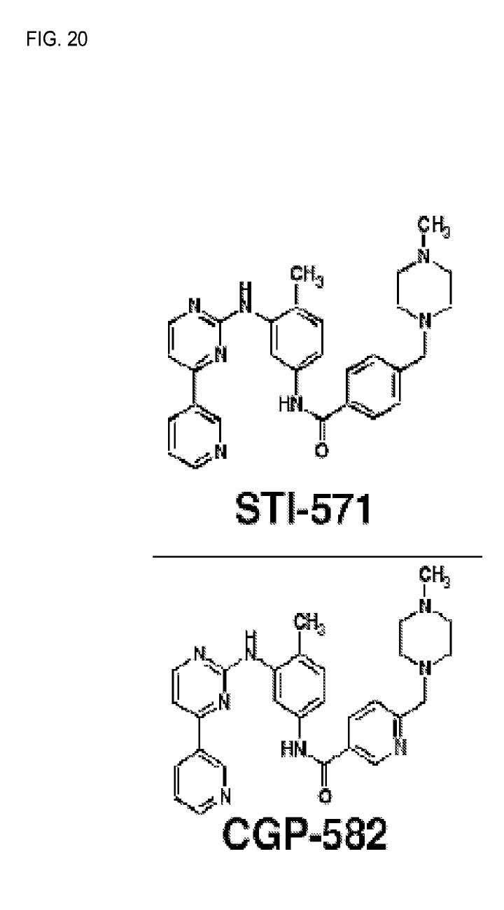

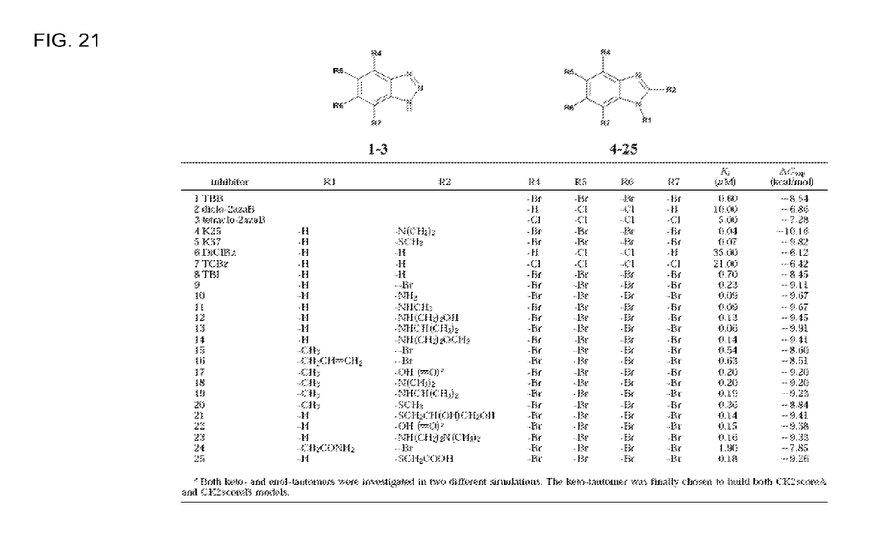

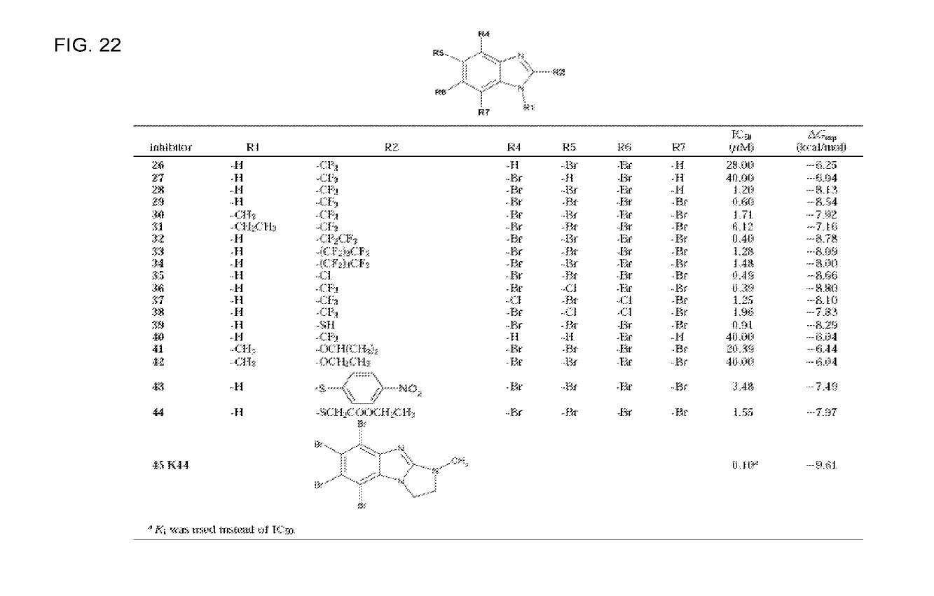

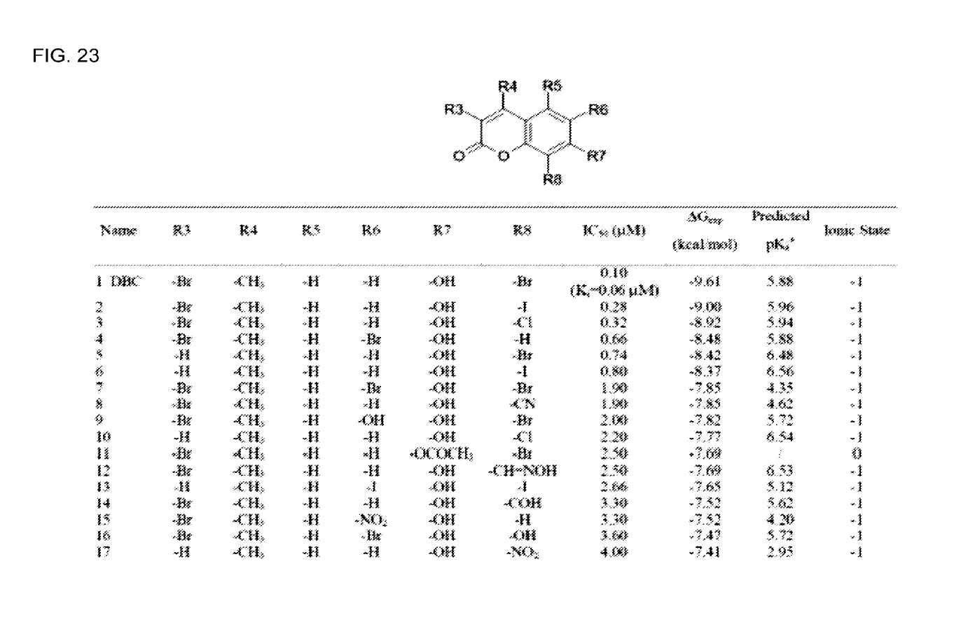

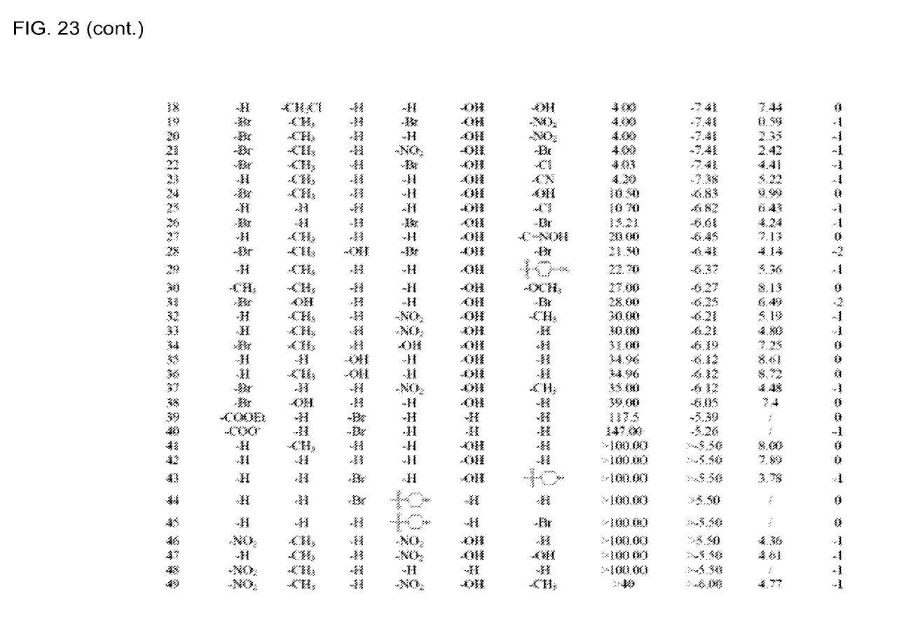

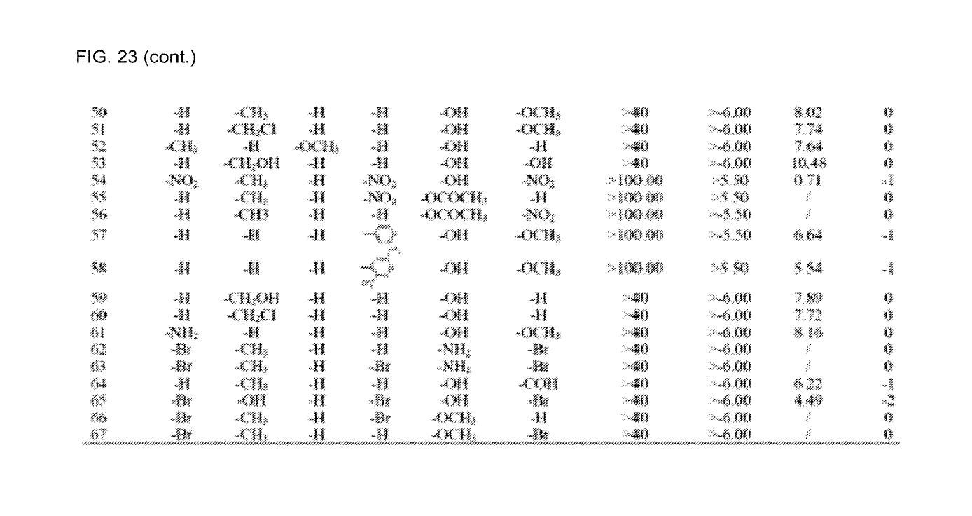

In some embodiments, applicable CK2 inhibiting agents include any of the compounds shown in FIGS. 15-23 (Bortolato, Andrea 31 Jan. 2008 Universita Degli Studi Di Padova—Doctoral Thesis “Rational Design of New Protein Kinases Inhibitors of Pharmaceutical Interest).

In some embodiments, applicable CK2 inhibiting agents include

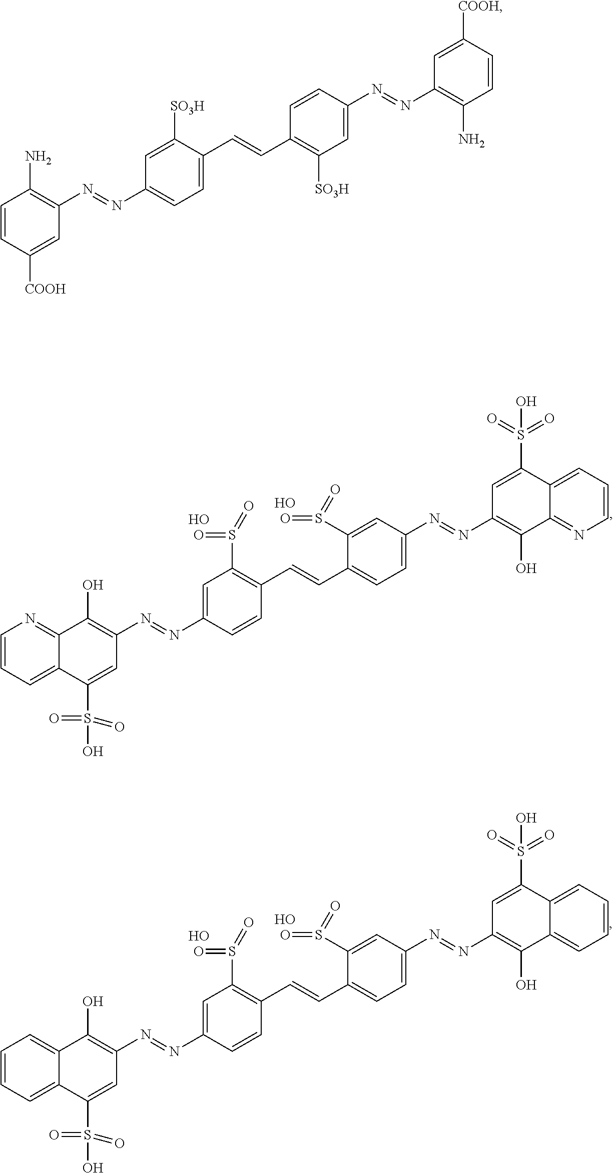

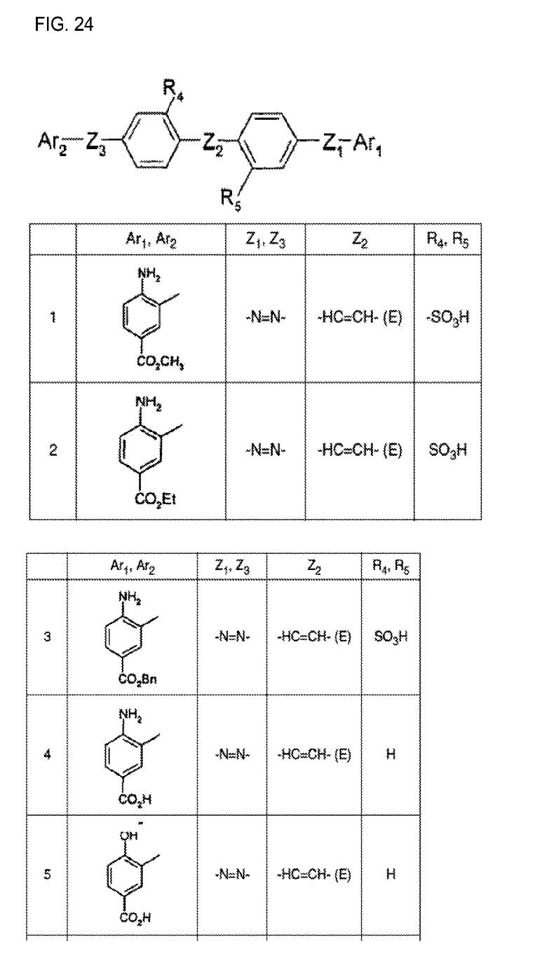

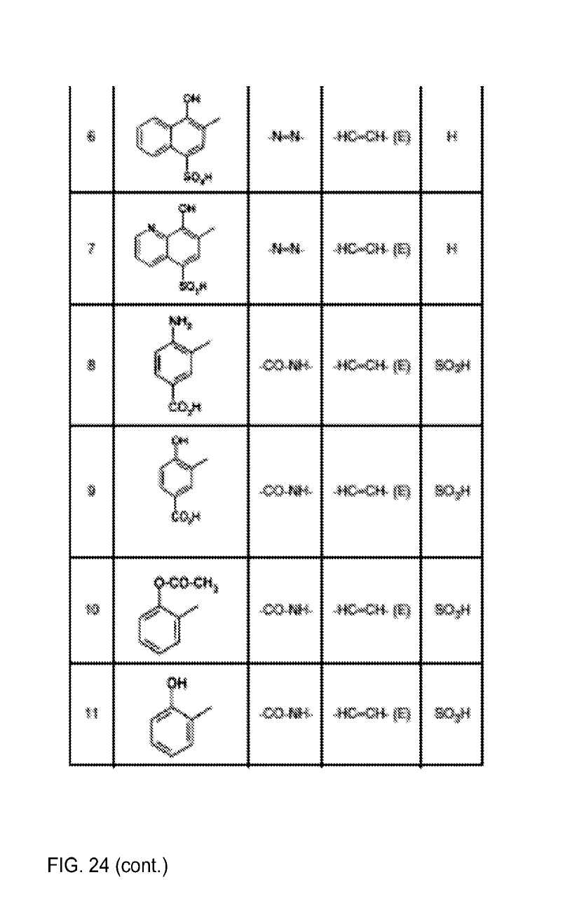

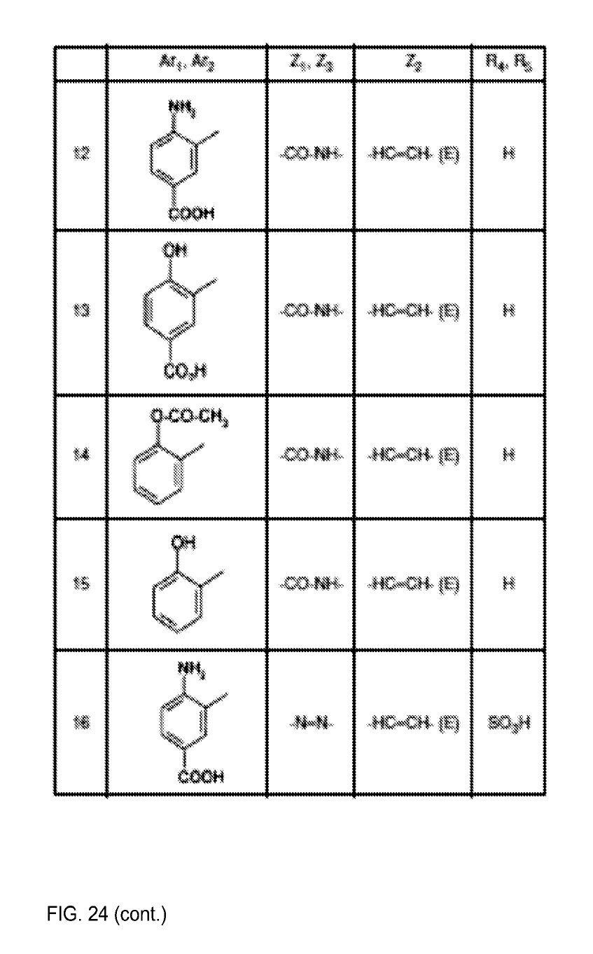

and any of the compounds shown in FIG. 24 (EP1911451).

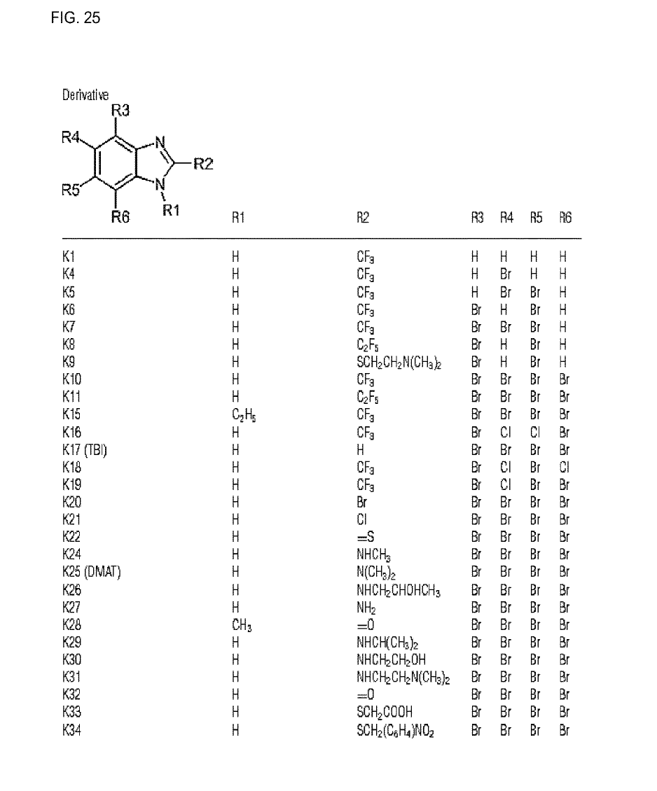

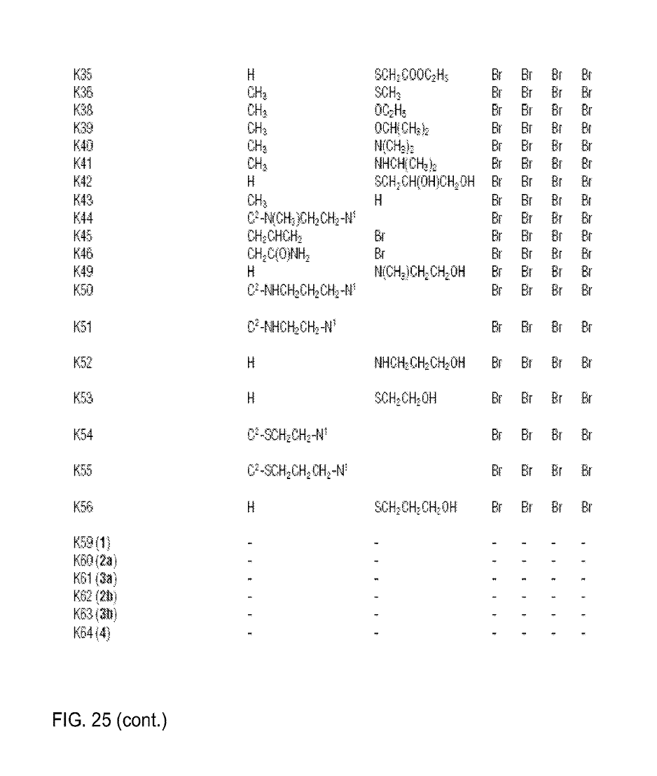

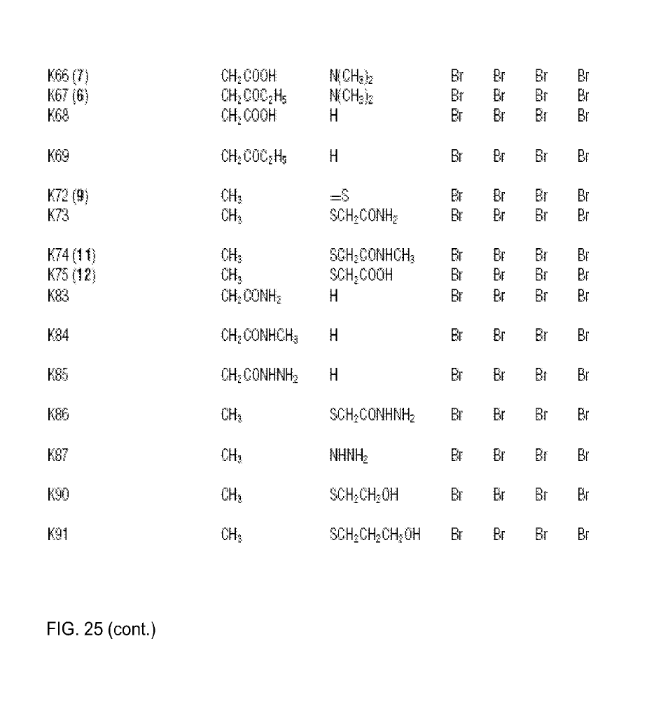

In some embodiments, applicable CK2 inhibiting agents include any of the compounds shown in FIG. 25 (Pagano, et al., 2008 Biochem. J. 415:353-365).

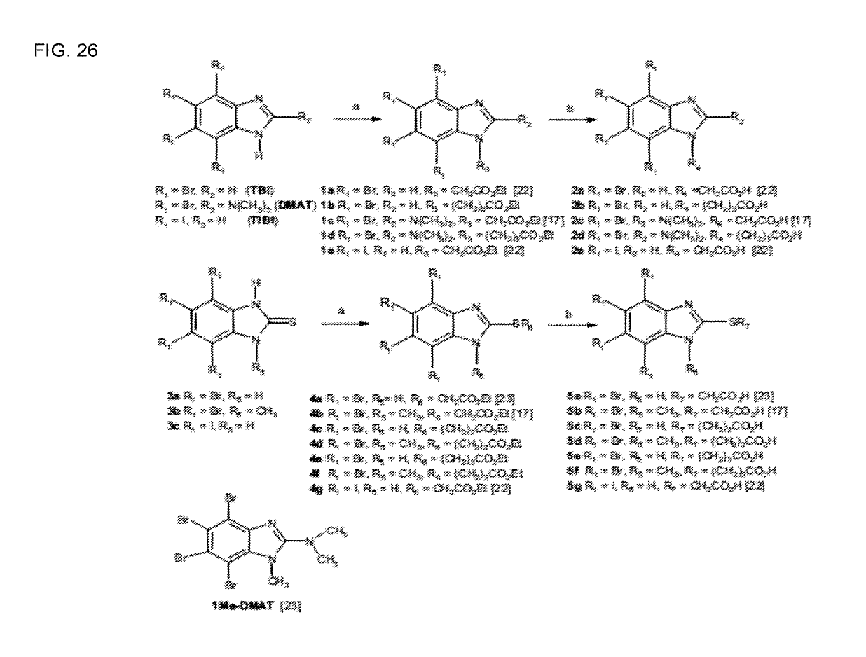

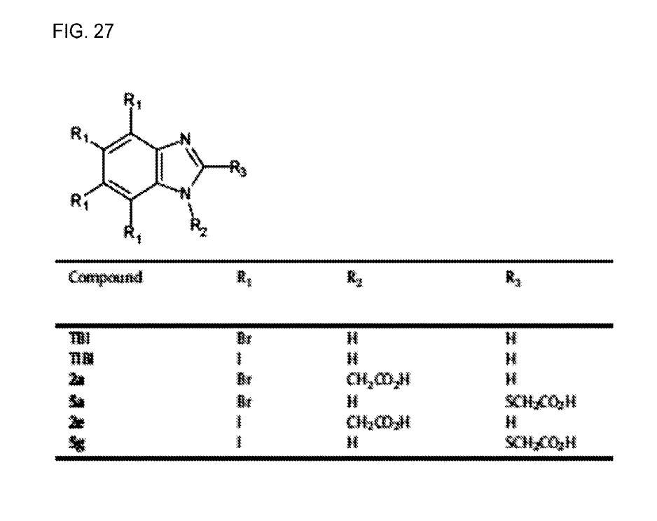

In some embodiments, applicable CK2 inhibiting agents include any of the compounds shown in FIGS. 26 and 27 (Janeczko, et al., 2012 European J. Med. Chem. 47:345-350).

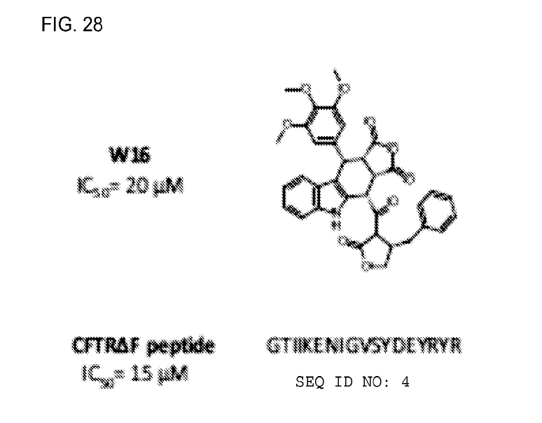

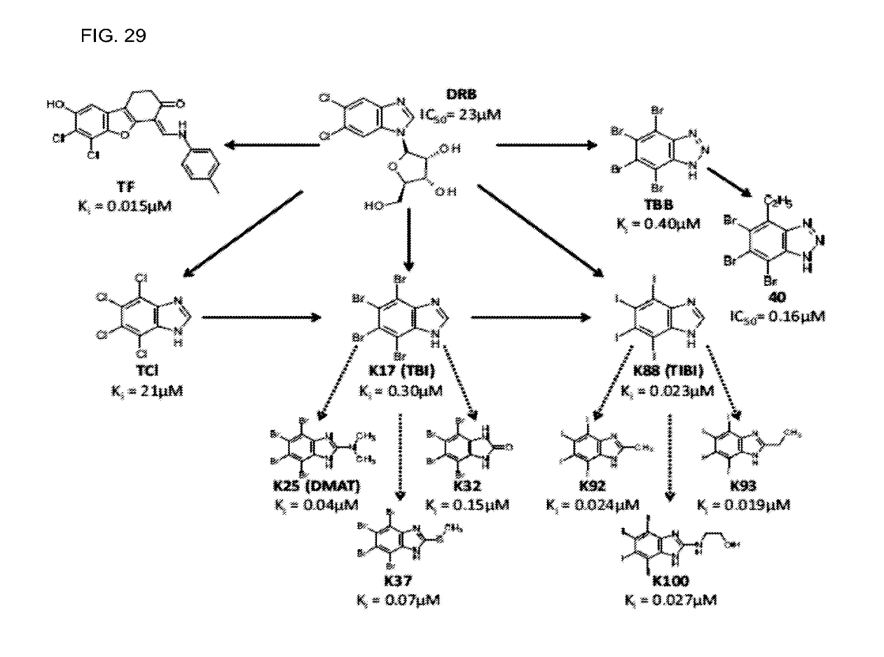

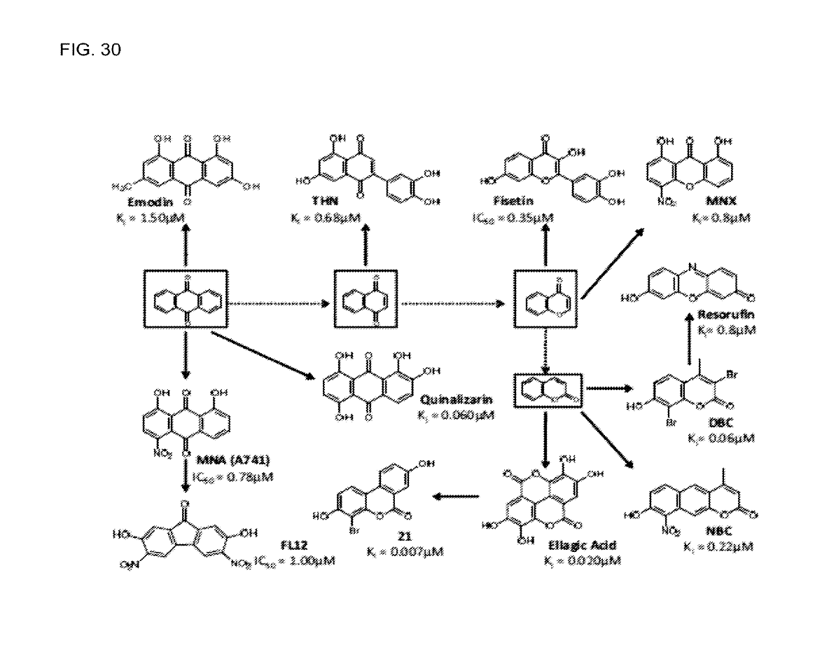

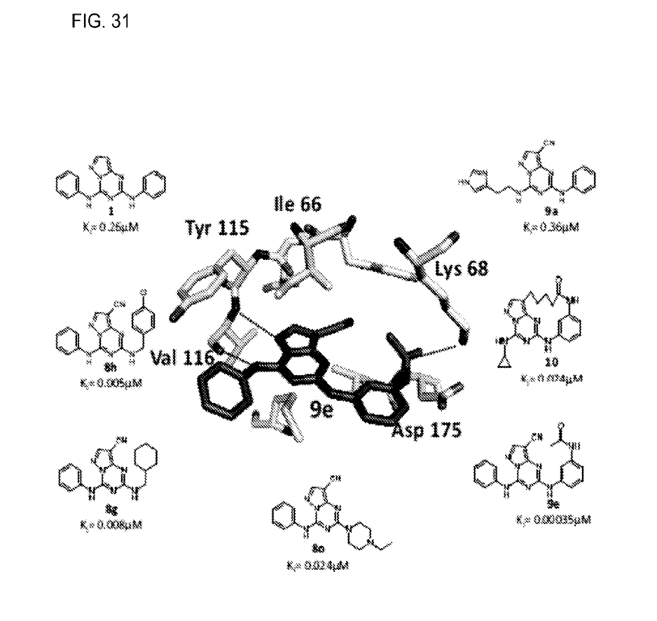

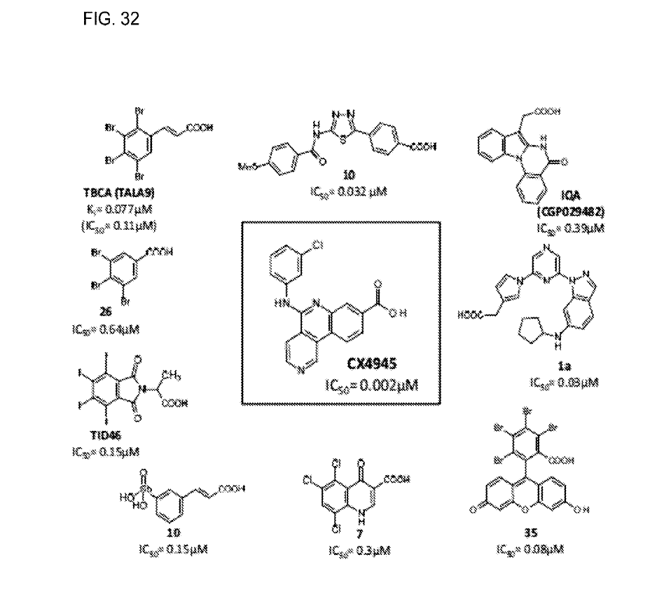

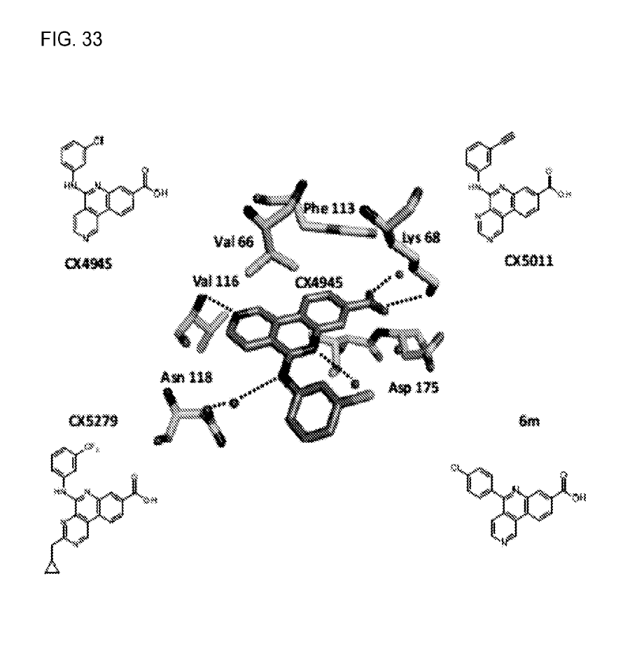

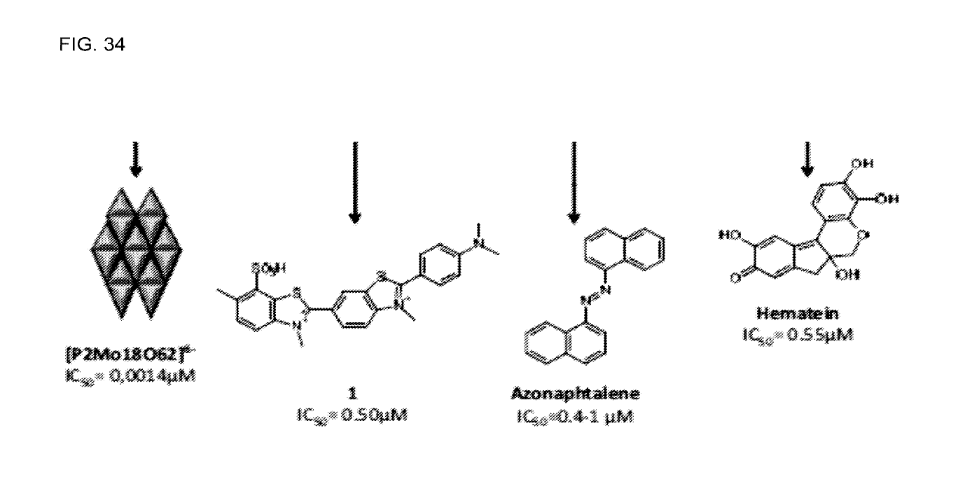

In some embodiments, applicable CK2 inhibiting agents include any of the compounds shown in FIGS. 28, 29, 30, 31, 32, 33 and 34 (Cozza, et al., 2013 Current Med. Chem. 20:671-693).

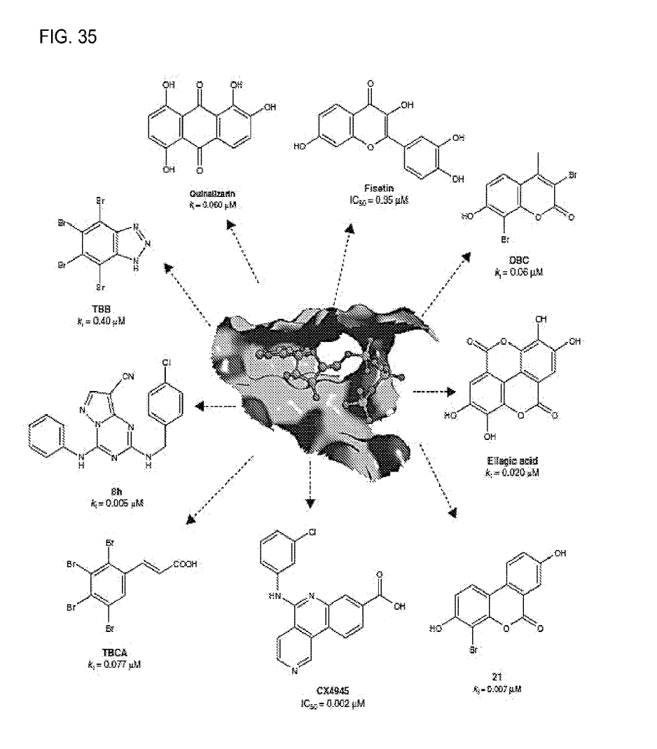

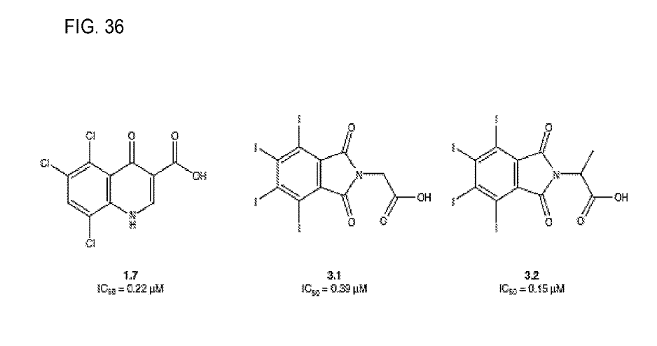

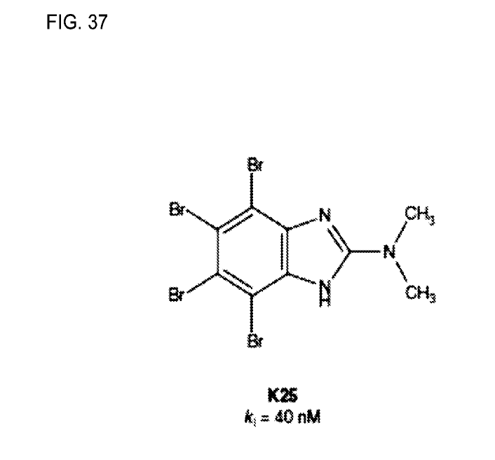

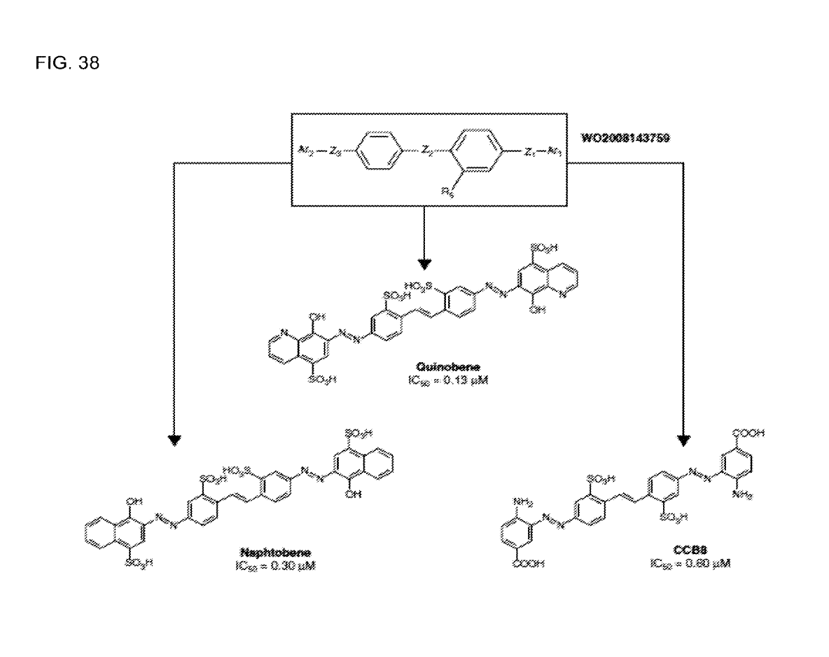

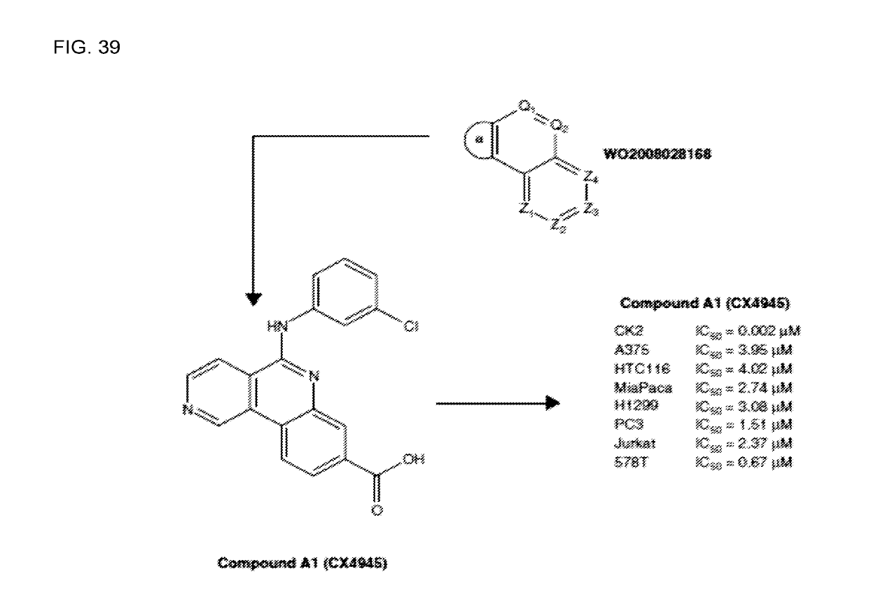

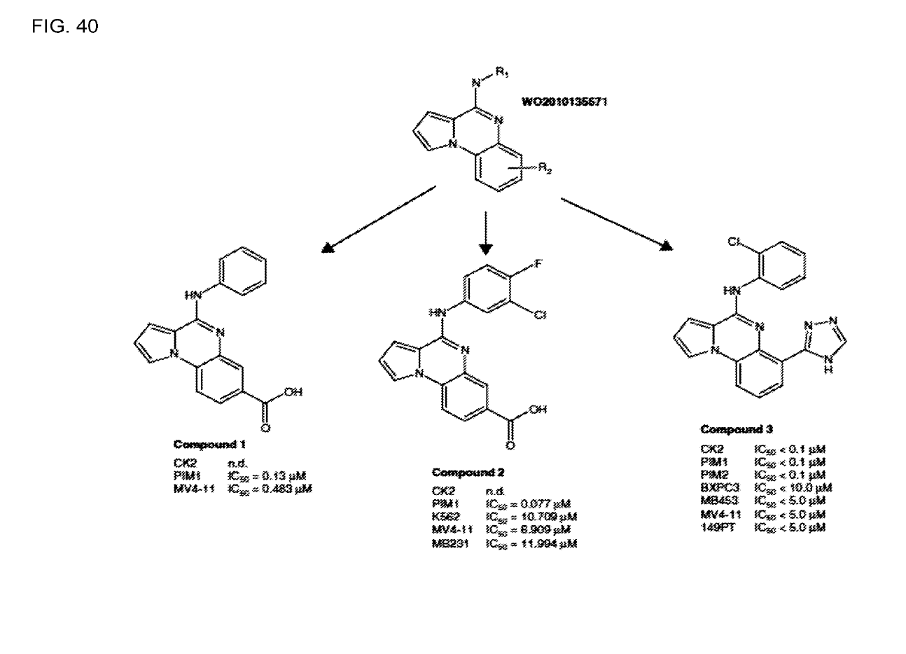

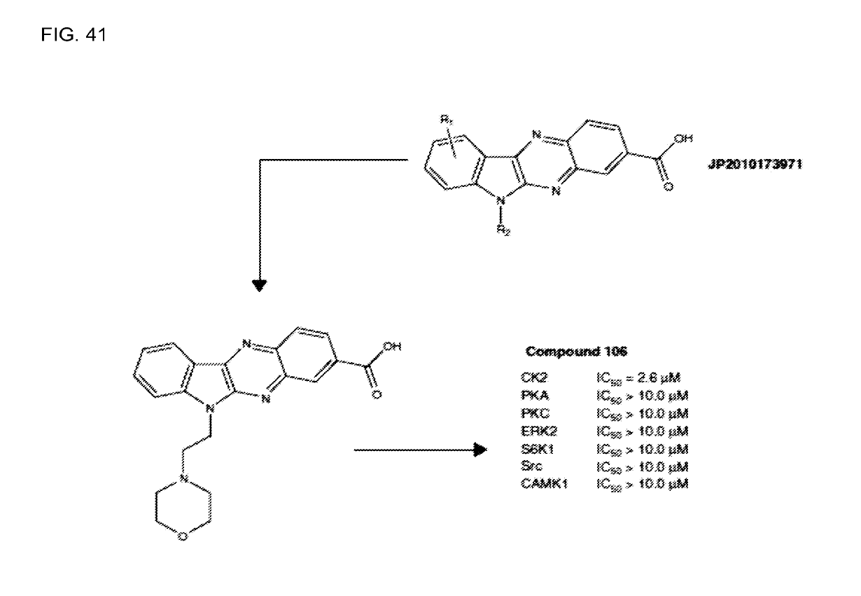

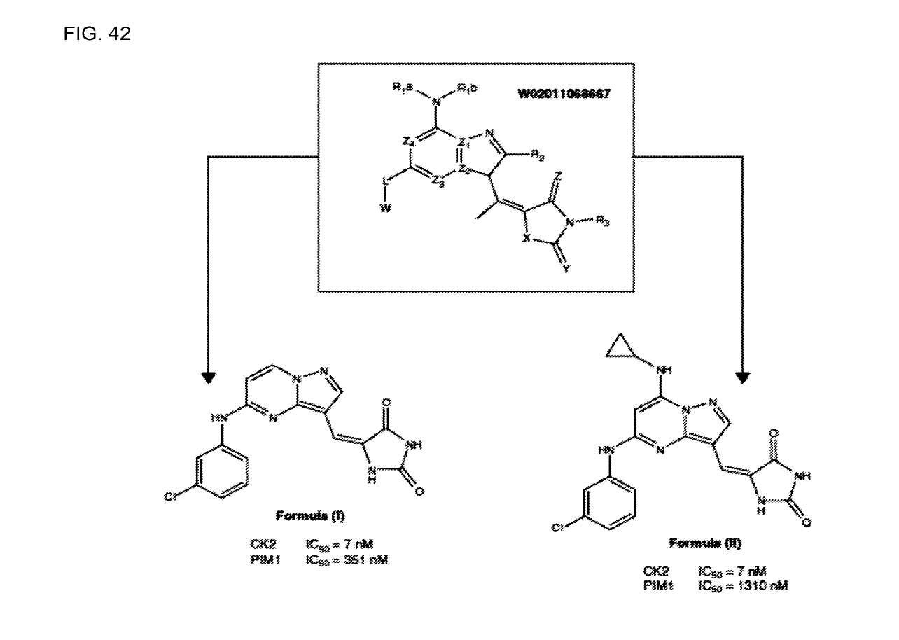

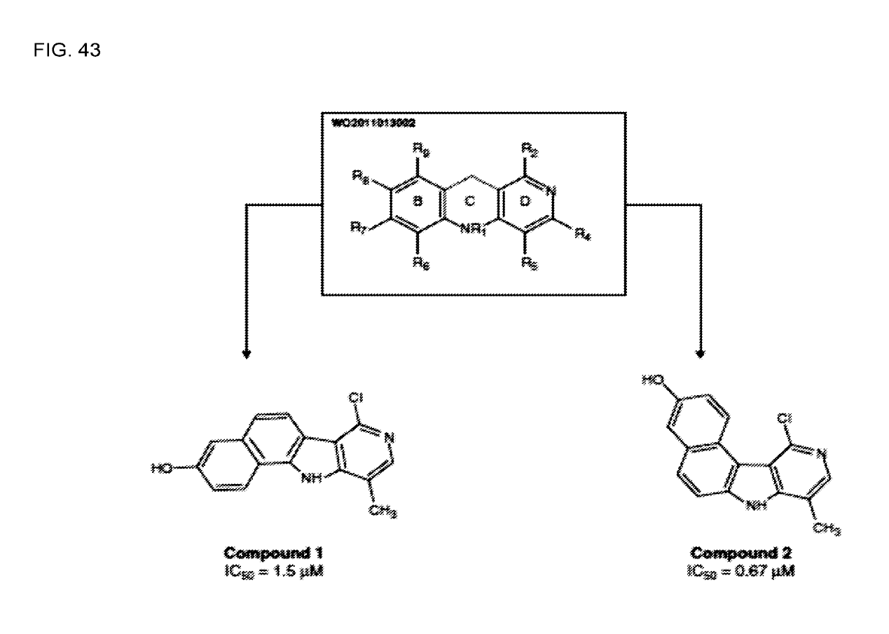

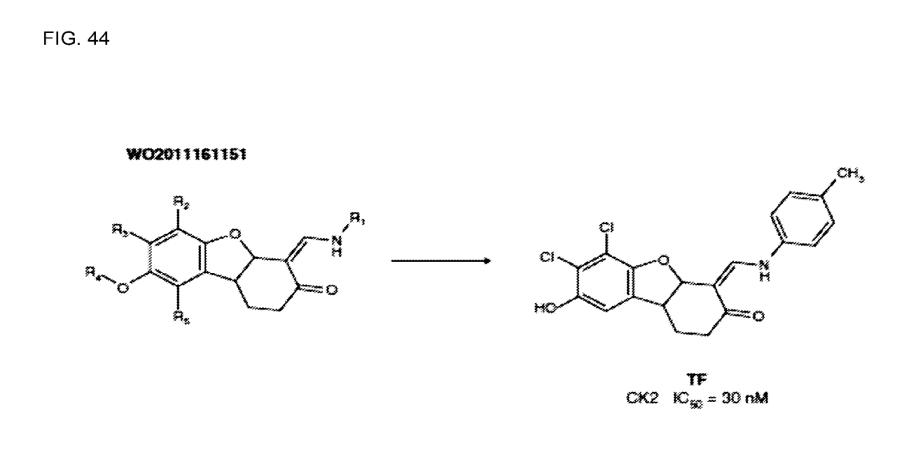

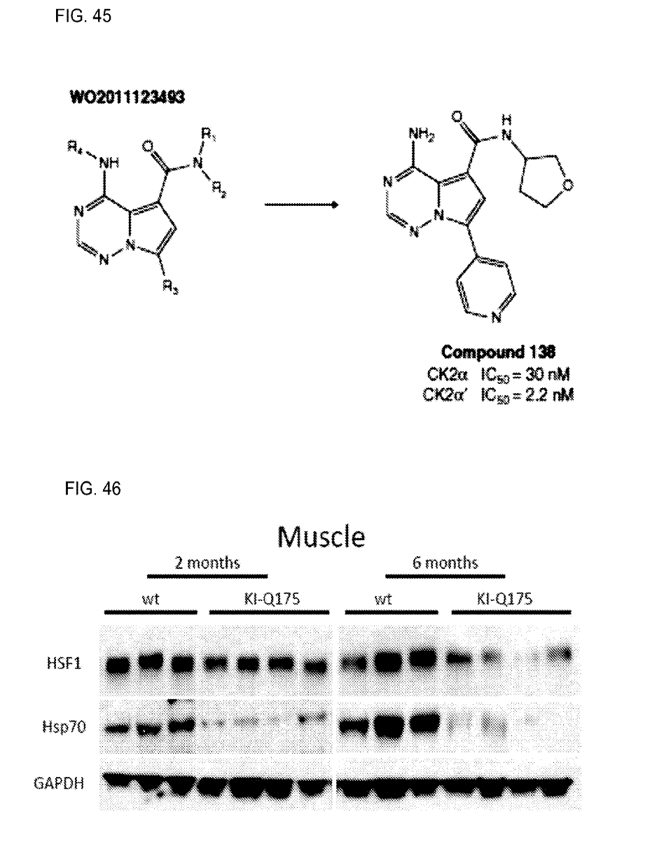

In some embodiments, applicable CK2 inhibiting agents include any of the compounds shown in FIGS. 35-45 (Cozza, et al., 2012 Expert Opin. Ther. Patents 22(9):1081-1097).

In some embodiments, applicable CK2 inhibiting agents are directed toward inhibiting CK2α and/or CK2α′ (e.g., tetrahalogenobenzimidazoles, including derivatives, 4,5,6,7-tetrabromo- and 4,5,6,7-tetraiodo-1H-benzimidazoles and N¹- and 2-S-carboxyalkyl derivatives) (Janeczko, et al., 2012 Euro. J. Med. Chem. 47:354-50).

In some embodiments, applicable CK2 inhibiting agents include any of the compounds shown in U.S. Patent Application Publication No. 2013/0023514, which include, for example:

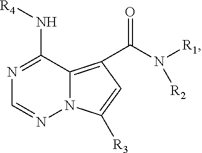

or a pharmaceutically acceptable salt thereof, wherein R₁ is selected from H, NRₐRₐ, C₁₋₆alkyl substituted with 0-5 R₁ₐ, C₂₋₆alkenyl substituted with 0-5 R₁ₐ, C₂₋₆alkynyl substituted with 0-5 R₁ₐ, —(CHR)ᵣ-carbocyclyl substituted with 0-5 R₁ₐ, —(CHR)ᵣ-heterocyclyl substituted with 0-5 R₁ₐ; R₁ₐ, at each occurrence, is independently selected from C₁₋₆alkyl substituted with 0-5 Rₑ, C₂₋₆alkenyl substituted with 0-5 Rₑ, C₂₋₆alkynyl substituted with 0-5 Rₑ, C₁₋₆haloalkyl, F, Cl, Br, NO₂, CN, ═O, —(CHR)ᵣOH, —(CHR)ᵣSH, (CHR)ᵣOR_{b}, —(CHR)ᵣS(O)ₚR_{b}, —(CHR)ᵣC(O)R_{d}, —(CHR)ᵣNRₐRₐ, —(CHR)ᵣC(O)NRₐRₐ, —(CHR)ᵣC(O)NRₐNRₐRₐ, —(CHR)ᵣNRₐC(O)R_{d}, —(CHR)ᵣNRₐC(O)OR_{b}, —(CHR)ᵣNRₐC(O)(CRR)ᵣOC(O)NRₐRₐ, —(CHR)ᵣNRₐC(O)(CRR)ᵣNRₐRₐ, —(CHR)NRₐC(O)(CRR)ᵣNRₐC(O)OR_{d}, —(CHR)ᵣOC(O)NRₐRₐ, —(CHR)ᵣC(O)OR_{d}, —(CHR)ᵣS(O)ₚNRₐRₐ, —(CHR)ᵣNRₐS(O)ₚR_{b}, —(CHR)ᵣ-carbocyclyl substituted with 0-5 Rₑ and —(CHR)ᵣ-heterocyclyl substituted with 0-5 Rₑ; R₂ is selected from H and C₁₋₆alkyl substituted with 0-3 R₂ₐ; R₂ₐ is selected from F, Cl, and Br; alternatively, R₁ and R₂ are taken together with the nitrogen atom to which they are attached to form a heterocyclyl substituted with 0-5 R₁ₐ; R₃ is selected from aryl substituted with 0-5 R₃ₐ and heteroaryl substituted with 0-5 R₃ₐ; R₃ₐ, at each occurrence, is independently selected from C₁₋₆alkyl, C₂₋₆alkenyl, C₂₋₆alkynyl, C₁₋₆haloalkyl, F, Cl, Br, NO₂, CN, —OH, —SH, —OR_{b}, —S(O)ₚR_{b}, C(O)R_{d}, —NRₐRₐ, —C(O)NRₐRₐ, —NRₐC(O)R_{d}, —NRₐC(O)OR_{b}, —OC(O)NRₐRₐ, —C(O)OR_{d}, —S(O)ₚNRₐRₐ, —NRₐS(O)ₚR_{b}; R₄ is selected from H, C₁₋₆alkyl, and C₃₋₆cycloalkyl; Rₐ, at each occurrence, is independently selected from H, NH₂, C₁₋₆alkyl substituted with 0-3 Rₑ, C₂₋₆alkenyl substituted with 0-3 Rₑ, C₂₋₆ alkynyl substituted with 0-3 Rₑ, C₁₋₆haloalkyl, —(CH₂)ᵣOH, (CH₂)ᵣ-carbocyclyl substituted with 0-3 Rₑ, and (CH₂)ᵣ-heterocyclyl substituted with 0-3 Rₑ, or Rₐ and Rₐ together with the nitrogen atom to which they are attached form a heterocyclyl substituted with 0-3 Rₑ; R_{b}, at each occurrence, is independently selected from C₁₋₆alkyl substituted with 0-3 Rₑ, C₁₋₆haloalkyl, C₂₋₆alkenyl substituted with 0-3 Rₑ, C₂₋₆alkynyl substituted with 0-3 Rₑ, —(CH₂)ᵣ-carbocyclyl substituted with 0-3 Rₑ, and —(CH₂)ᵣ-heterocyclyl substituted with 0-3 Rₑ; R_{d}, at each occurrence, is independently selected from H, C₁₋₆alkyl substituted with 0-3 Rₑ, C₁₋₆haloalkyl, C₂₋₆alkenyl substituted with 0-3 Rₑ, C₂₋₆alkynyl substituted with 0-3 Rₑ, —(CH₂)ᵣ-carbocyclyl substituted with 0-3 Rₑ, and —(CH₂)ᵣ-heterocyclyl substituted with 0-3 Rₑ; Rₑ, at each occurrence, is independently selected from C₁₋₆alkyl, C₂₋₆alkenyl, C₂₋₆alkynyl, (CH₂)ᵣC₃₋₆cycloalkyl, F, Cl, Br, CN, NO₂, CO₂H, ═O, —C(O)NR_{f}R_{f}, (CF₂)ᵣCF₃, —(CH₂)ᵣOC₁₋₅alkyl, —(CH₂)ᵣOH, SH, —(CH₂)ᵣSC₁₋₅alkyl, —(CH₂)ᵣNR_{f}R_{f}, —(CH₂)ᵣphenyl, and (CH₂)ᵣheterocyclyl; R_{f}, at each occurrence, is independently selected from H, C₁₋₅alkyl, C₃₋₆cycloalkyl, and phenyl; R, at each occurrence, is independently selected from H, —(CH₂)ᵣOH, C₁₋₆alkyl, C₁₋₆haloalkyl, and —(CH₂)ᵣ-aryl; p, at each occurrence, is independently selected from 0, 1, and 2; and r, at each occurrence, is independently selected from 0, 1, 2, 3, and 4.

In some embodiments, applicable CK2 inhibiting agents include any of the compounds shown in U.S. Pat. No. 8,575,177 (and all related cases). For example, in some embodiments, the CK2 inhibiting agent is encompassed within Formula I of U.S. Pat. No. 8,575,177:

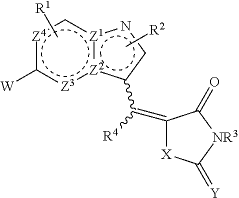

wherein the bicyclic ring system containing Z¹-Z⁴ is aromatic;
one of Z¹ and Z² is C, the other of Z¹ and Z² is N;
Z³ and Z⁴ are independently CR⁵ or N,
   where R⁵ can be H or R¹;
R¹ is H, halo, CN, optionally substituted C1-C4 alkyl, optionally substituted C2-C4 alkenyl, optionally substituted C2-C4 alkynyl, optionally substituted C1-C4 alkoxy, or —NR⁷R⁸,
where R⁷ and R⁸ are each independently selected from H, optionally substituted C1-C10 alkyl, optionally substituted aryl, optionally substituted arylalkyl, optionally substituted heteroaryl, and optionally substituted heteroarylalkyl,
or R⁷ and R⁸ taken together with the N of —NR²R⁸ form an optionally substituted 5-8 membered ring that optionally contains an additional heteroatom selected from N, O and S as a ring member;
R² is H, halo, CN, or an optionally substituted group selected from C1-C4 alkyl, C2-C4 alkenyl, and C2-C4 alkynyl;
R³ and R⁴ are independently selected from H and optionally substituted C1-C10 alkyl;
X is NR⁶, O, or S, where R⁶ is H or an optionally substituted group selected from C1-C4 alkyl, C2-C4 alkenyl, and C2-C4 alkynyl;
Y is O or S;

W is optionally substituted aryl, optionally substituted heteroaryl, or —NR⁹R¹⁰, —OR⁹, S(O)ₙR⁹, optionally substituted carbon-linked heterocyclyl, optionally substituted C3-C8 cycloalkyl, or CR⁹R¹⁰R¹¹,
wherein n is 0, 1 or 2, and
R⁹ and R¹⁰ are each independently selected from H, optionally substituted C1-C10 alkyl, optionally substituted aryl, optionally substituted arylalkyl, optionally substituted heteroaryl, and optionally substituted heteroarylalkyl,
   or R⁹ and R¹⁰ taken together with the N of —NR⁹R¹⁰ form an optionally substituted 5-8 membered ring that optionally contains an additional heteroatom selected from N, O and S as a ring member, and
R¹¹ is selected from H, optionally substituted C1-C10 alkyl, optionally substituted aryl, optionally substituted arylalkyl, optionally substituted heteroaryl, and optionally substituted heteroarylalkyl.

In some embodiments, the CK2 inhibiting agent is encompassed within Formula II or Formula II′ of U.S. Pat. No. 8,575,177:

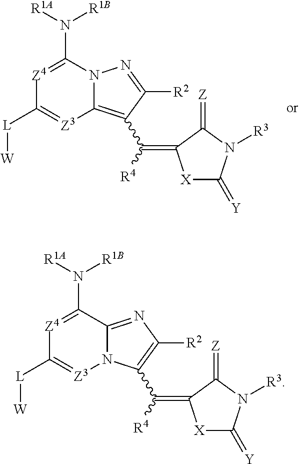

wherein:
Z³ and Z⁴ each independently represent N or CR⁵, or CH;
   each R⁵ is independently selected from halo, CN, R, —OR, —S(O)ₙR, COOR, CONR², and NR₂,
   wherein each R is independently selected from H and optionally substituted C1-C4 alkyl, and the two R groups of NR₂ can be linked together to form a 5-6 membered heterocyclic ring that is optionally substituted and can include an additional heteroatom selected from N, O and S as a ring member;
R², R³ and R⁴ are each independently selected from H and optionally substituted C1-C10 alkyl;
X represents O, S, or NR²;
Y is O or S or NR¹⁰;
   where R¹⁰ is selected from H, CN, optionally substituted C1-C4 alkyl, optionally substituted C2-C4 alkenyl, optionally substituted C2-C4 alkynyl, optionally substituted C1-C4 alkoxy, and —NR⁷R⁸,
Z is O or S;
   L can be a bond, —CR⁷═CR⁸—, —C≡C—, —NR⁷—, —O—, —S(O)ₙ—, or (CR⁷R⁸)ₘ, —(CR⁷R⁸)ₘ—NR⁷—, —(CR⁷R⁸)ₘ—O—, or —(CR⁷R⁸)ₘ—S(O)ₙ—;
   W is optionally substituted C1-C10 alkyl, optionally substituted aryl, optionally substituted heteroaryl, —NR⁷R⁸, —OR⁷, S(O)ₙR⁷, CONR⁷R⁸, optionally substituted heterocyclyl, optionally substituted C3-C8 cycloalkyl, optionally substituted C2-C10 alkenyl, optionally substituted C2-C10 alkynyl, or CR⁷R⁸R⁹,
   where each R⁷ and R⁸ and R⁹ is independently selected from H, optionally substituted C1-C6 alkoxy, optionally substituted C1-C6 alkylamino, optionally substituted C1-C6 dialkylamino, optionally substituted heterocyclyl, optionally substituted C1-C10 alkyl, optionally substituted C3-C8 cycloalkyl, optionally substituted C4-C10 cycloalkylalkyl, optionally substituted aryl, optionally substituted arylalkyl, optionally substituted heteroaryl, and optionally substituted heteroarylalkyl;
   or R⁸ and R⁹ taken together can be ═O (oxo) or ═N—OR⁷ or ═N—CN;
   or R⁷ and R⁸ taken together with the N of —NR⁷R⁸ can form an optionally substituted 5-10 membered heterocyclic or heteroaromatic ring system that optionally contains an additional heteroatom selected from N, O and S as a ring member;
   provided that no more than one of or R⁷ and R⁸ in —NR⁷R⁸ is selected from the group consisting of alkoxy, alkylamino, dialkylamino and heterocyclyl;
each n is independently is 0, 1 or 2;
each m is independently 1, 2, 3 or 4;
   R^{1A} and R^{1B} are each independently selected from H, optionally substituted C1-C10 alkyl, optionally substituted heterocyclyl, optionally substituted cycloalkyl, optionally substituted cycloalkylalkyl, optionally substituted heterocyclylalkyl, optionally substituted arylalkyl, or an optionally substituted 5-6 membered aryl ring containing up to two heteroatoms as ring members;
   or R^{1A} and R^{1B} in —NR^{1A}R^{1B} can be taken together to form an optionally substituted 5-8 membered monocyclic or 5-10 membered bicyclic heteroaryl or heterocyclic group containing up to two additional heteroatoms selected from N, O and S as ring members;
and pharmaceutically acceptable salts of these compounds.

In some embodiments, the CK2 inhibiting agent is encompassed within Formula IIa or

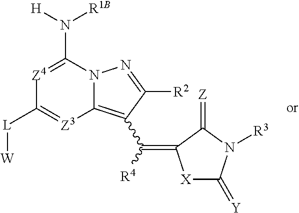

Formula IIa′ of U.S. Pat. No. 8,575,177:

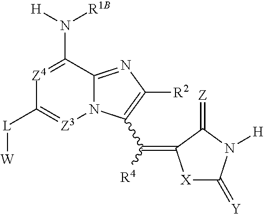

where R^{Th} is selected from H, halo, optionally substituted C1-C6 alkyl, CN, S(O)₀₋₂R, —SO₂NR₂, COOR, CONR₂, and C(O)R,
where each R is independently H, halo, CN, or an optionally substituted member selected from the group consisting of C1-C6 alkyl, C1-C6 alkoxy, C1-C6 alkylamino, di(C1-C6)alkylamino, C3-C8 cycloalkyl, C4-C10 cycloalkylalkyl, C5-C8 heterocyclyl, C6-C10 heterocyclylalkyl, aryl, arylalkyl, C5-C6 heteroalkyl, and C6-C10 heteroalkylalkyl;
and two R on the same atom or adjacent atoms can form an optionally substituted heterocyclic ring that can contain an additional heteroatom selected from N, O and S; and other structural features are as defined for Formula IIa above.
In some embodiments, the CK2 inhibiting agent is encompassed within Formula II-Th or

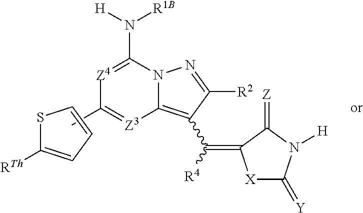

Formula II-Th′ of U.S. Pat. No. 8,575,177

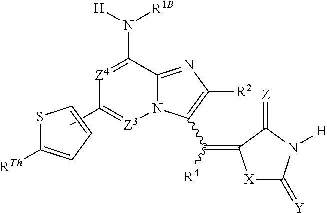

where R^{Th} is selected from H, halo, optionally substituted C1-C6 alkyl, CN, S(O)₀₋₂R, —SO₂NR₂, COOR, CONR₂, and C(O)R,
where each R is independently H, halo, CN, or an optionally substituted member selected from the group consisting of C1-C6 alkyl, C1-C6 alkoxy, C1-C6 alkylamino, di(C1-C6)alkylamino, C3-C8 cycloalkyl, C4-C10 cycloalkylalkyl, C5-C8 heterocyclyl, C6-C10 heterocyclylalkyl, aryl, arylalkyl, C5-C6 heteroalkyl, and C6-C10 heteroalkylalkyl;
and two R on the same atom or adjacent atoms can form an optionally substituted heterocyclic ring that can contain an additional heteroatom selected from N, O and S; and other structural features are as defined for Formula IIa above.

In some embodiments, applicable CK2 inhibiting agents include any of the compounds shown in China 201410228164.X entitled, “Pyrazolopyrimidine Prodrugs and Methods Of Use” (and all related cases).

The CK2 inhibiting agents described in the present invention are useful in the preparation of pharmaceutical formulation, also synonymously referred to herein as “medicaments,” to treat a variety of conditions associated with protein misfolding and/or reduced HSF1 activity. In addition, the CK2 inhibiting agents are also useful for preparing medicaments for treating other disorders wherein the effectiveness of the CK2 inhibiting agents are known or predicted. Such disorders include, but are not limited to, neurological disorders. The methods and techniques for preparing medicaments of a CK2 inhibiting agent described in the present invention are well-known in the art. Exemplary pharmaceutical formulations and routes of delivery are described below.

One of skill in the art will appreciate that any one or more of the CK2 inhibiting agents described herein, including the many specific embodiments, are prepared by applying standard pharmaceutical manufacturing procedures. Such pharmaceutical formulations can be delivered to the subject by using delivery methods that are well-known in the pharmaceutical arts.

In some embodiments of the present invention, the compositions are administered alone, while in some other embodiments, the compositions are preferably present in a pharmaceutical formulation comprising at least one active ingredient/agent, as defined above, together with a solid support or alternatively, together with one or more pharmaceutically acceptable carriers and optionally other therapeutic agents. Each carrier must be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

In some embodiments, tablets comprise at least one active ingredient and optionally one or more accessory agents/carriers are made by compressing or molding the respective agents. In some embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternatively embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in a known manner. This phase typically comprises a lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate. Likewise, those for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent. It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

In some embodiments, the CK2 inhibiting agents described in the present invention are provided in unsolvated form or are in non-aqueous solutions (e.g., ethanol). The inhibiting agents may be generated to allow such formulations through the production of specific crystalline polymorphs compatible with the formulations.

In certain embodiments, the present invention provides instructions for administering said CK2 inhibiting agents to a subject. In certain embodiments, the present invention provides instructions for using the compositions contained in a kit for the treatment of conditions characterized by the dysregulation of apoptotic processes in a cell or tissue (e.g., providing dosing, route of administration, decision trees for treating physicians for correlating patient-specific characteristics with therapeutic courses of action). In certain embodiments, the present invention provides instructions for using the compositions contained in the kit to treat a variety of medical conditions associated with reduced HSF1 activity (e.g., medical conditions involving irregular HSF1 activity) (e.g., medical conditions involving irregular chaperone activity) (e.g., Alzheimer's disease, Parkinson's disease, Huntington's disease, Amyotrophic Lateral Sclerosis, prion-based diseases, type 1 diabetes mellitus, type 2 diabetes mellitus).

Various delivery systems are known and can be used to administer therapeutic agents (e.g., the CK2 inhibiting agents described in the present invention) of the present invention, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

It is contemplated that the agents identified can be administered to subjects or individuals susceptible to or at risk of developing pathological growth of target cells and correlated conditions. When the agent is administered to a subject such as a mouse, a rat or a human patient, the agent can be added to a pharmaceutically acceptable carrier and systemically or topically administered to the subject. To determine patients that can be beneficially treated, a tissue sample is removed from the patient and the cells are assayed for sensitivity to the agent.

Therapeutic amounts are empirically determined and vary with the pathology being treated, the subject being treated and the efficacy and toxicity of the agent. When delivered to an animal, the method is useful to further confirm efficacy of the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the CK2 inhibiting agents provided herein are administered at about 0.01 mg/kg to about 200 mg/kg, more preferably at about 0.1 mg/kg to about 100 mg/kg, even more preferably at about 0.5 mg/kg to about 50 mg/kg. When the CK2 inhibiting agents described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or non-aqueous diluents, syrups, granulates or powders. In addition to a CK2 inhibiting agent described in the present invention, the pharmaceutical compositions can also contain other pharmaceutically active compounds or a plurality of CK2 inhibiting agents described in the present invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

The present invention also includes methods involving co-administration of the CK2 inhibiting agents described herein with one or more additional active agents. Indeed, it is a further aspect of this invention to provide methods for enhancing prior art therapies and/or pharmaceutical compositions by co-administering a CK2 inhibiting agent described herein. In co-administration procedures, the agents may be administered concurrently or sequentially. In one embodiment, the CK2 inhibiting agents described herein are administered prior to the other active agent(s). The pharmaceutical formulations and modes of administration may be any of those described above. In addition, the two or more co-administered chemical agents, biological agents or radiation may each be administered using different modes or different formulations.

The agent or agents to be co-administered depends on the type of condition being treated. For example, when the condition being treated is a neurological disorder (e.g., Huntington Disease), the additional agent can be an anticonvulsant medication. The additional agents to be co-administered can be any of the well-known agents in the art for a particular disorder, including, but not limited to, those that are currently in clinical use and/or experimental use.

In certain embodiments, the present invention provides methods (e.g., therapeutic applications) for treating conditions associated with protein misfolding. The present invention is not limited to a particular type of method. In some embodiments, the methods for treating conditions associated with protein misfolding comprise a) providing: i. target cells having misfolded proteins; and ii. a composition (e.g., a composition comprising a CK2 inhibiting agent); and b) exposing the target cells to the composition under conditions such that the exposure results in increased HSF1 activity.

The methods are not limited to treating a particular condition associated with protein misfolding. In some embodiments, the condition associated with protein misfolding is a medical condition involving irregular chaperone activity. In some embodiments, the condition associated with protein misfolding is enhanced aging, Alzheimer's disease, Parkinson's disease, Huntington disease, Amyotrophic Lateral Sclerosis, and prion-based disease (e.g., transmissible spongiform encephalopathy, Bovine spongiform encephalopathy, Creutzfeldt-Jakob disease, and Kuru). In some embodiments, the condition associated with protein misfolding is type 2 diabetes mellitus (see, e.g., Chung, J, et. al., PNAS 2008, 1739-1744).

In some embodiments, the condition involves aberrant HSF1 activity such as, for example, Alzheimer's disease, glaucoma, prion disease, tauopathies, fronto-temporal degeneration, Huntington's disease, Kennedy's disease, familial dementia, Cushing's disease, neurofibromatosis, some lysosomal storage diseases, diabetes, cataracts, cardiac atrial amyloidosis, Parkinson's disease, cystic fibrosis, and sickle cell disease. In some embodiments, the condition ameliorated by restoration of HSF1 activity is any type of proteopathy disorder. Examples of a proteopathy disorder include, but are not limited to, Alzeimer's disease, cerebral β-amyloid angiopathy, retinal ganglion cell degeneration in glaucoma, prion diseases, Parkinson's disease, synucleinopathies, tauopahties, frontotemporal lobar degeneration, amyotrophic lateral sclerosis, Huntington's disease, hereditary cerebral hemorrhage with amyloidosis, cerebral autosomal-dominant arteriopathy with subcortical infarcts and leukoencephalopathy, Alexander disease, seipinopathies, familial amyloidotic neuropathy, senile systemic amyloidosis, serpinopathies, light chain amyloidosis, heavy chain amyloidosis, secondary amyloidosis, type 2 diabetes, aortic medial amyloidosis, ApoAI amyloidosis, ApoAII amyloidosis, ApoAIV amyloidosis, familial amyloidosis of the Finnish type, lysozyme amyloidosis, fibrinogen amyloidosis, dialysis amyloidosis, inclusion body myositis/myopathy, cataracts, retinitis pigmentosa with rhodopsin mutations, medullary thyroid carcinoma, cardiac atrial amyloidosis, pituitary prolactinoma, hereditary lattice corneal dystrophy, cutaneous lichen amyloidosis, Mallory bodies, corneal lactoferrin amyloidosis, pulmonary alveolar proteinosis, odontogenic (Pinborg) tumor amyloid, seminal vesical amyloid, cystric fibrosis, sickle cell disease, and critical illness myopathy.

In some embodiments, the condition is a neurodegenerative disorder characterized by tandem amplification of tri-nucleotide repeats within respective protein coding regions (e.g., a trinucleotide repeat disorder). In some embodiments, the trinucleotide repeat disorder is a polyglutamine (PolyQ) disease (e.g., dentatorubropallidoluysian atrophy, Huntington's Disease, spinobulbar muscular atrophy (Kennedy disease), spinocerebellar ataxia type 1, spinocerebellar ataxia type 2, spinocerebellar ataxia type 3 (Machado-Joseph disease), spinocerebellar ataxia type 6, spinocerebellar ataxia type 7, and spinocerebellar ataxia type 17. In some embodiments, the trinucleotide repeat disorder is not a polyglutamine (PolyQ) disease (e.g., fragile X syndrome, fragile X-associated tremor/ataxia syndrome), fragile XE mental retardation, Friedreich's ataxia, myotonic dystrophy, spinocerebellar ataxia type 8, and spinocerebellar ataxia type12).

The methods are not limited to a particular type of target cells. In some embodiments, the target cells are neurological cells. In some embodiments, the target cells are within a living mammal (e.g., human, horse, dog, cat, pig, rat, mouse, ape, monkey)

Additionally, any one or more of these compounds can be used in combination with at least one other therapeutic agent (e.g., potassium channel openers, calcium channel blockers, sodium hydrogen exchanger inhibitors, anticonvulsant agents, antiarrhythmic agents, antiatherosclerotic agents, anticoagulants, antithrombotic agents, prothrombolytic agents, fibrinogen antagonists, diuretics, antihypertensive agents, ATPase inhibitors, mineralocorticoid receptor antagonists, phosphodiesterase inhibitors, antidiabetic agents, anti-inflammatory agents, antioxidants, angiogenesis modulators, antiosteoporosis agents, hormone replacement therapies, hormone receptor modulators, oral contraceptives, antiobesity agents, antidepressants, antianxiety agents, antipsychotic agents, antiproliferative agents, antitumor agents, antiulcer and gastroesophageal reflux disease agents, growth hormone agents and/or growth hormone secretagogues, thyroid mimetics, anti-infective agents, anti-spastic agents, antiviral agents, antibacterial agents, antifungal agents, cholesterol/lipid lowering agents and lipid profile therapies, and agents that mimic ischemic preconditioning and/or myocardial stunning, antiatherosclerotic agents, anticoagulants, antithrombotic agents, antihypertensive agents, antidiabetic agents, and antihypertensive agents selected from ACE inhibitors, AT-1 receptor antagonists, ET receptor antagonists, dual ET/AII receptor antagonists, and vasopepsidase inhibitors, or an antiplatelet agent selected from GPIIb/IIIa blockers, P2Y₁ and P2Y₁₂ antagonists, thromboxane receptor antagonists, and aspirin) in along with a pharmaceutically-acceptable carrier or diluent in a pharmaceutical composition. Additional therapeutic agents for Huntington disease include, but are not limited to, anticonvulsant agents (e.g., valproic acid, clonazepam), antipsychotic agents (e.g., risperidone, haloperidol), rauwolfia alkaloids (e.g., reserpine), antidepressants (e.g., proxetine). Additional therapeutic agents for Parkinson's disease include, but are not limited to, dopamine prodrugs (e.g., levodopa/carbidopa), dopamine agonists (e.g., apomorphine, bromocriptine, pergolide, pramipexole, ropinirole, rotigotine), catechol-O-methyltransferase (COMT) inhibitors (e.g., tolcapone, entacapone, levodopa, carbidopa, entacapone), anticholinergics (e.g., trihexyphenidyl, benztropine mesylate), MAO-B inhibitors (e.g., selegiline, rasagiline), and N-methyl-D-aspartic acid inhibitors (e.g., amantadine). Additional therapeutic agents for Alzheimer's disease include, but are not limited to, centrally acting AChE inhibitors (e.g., rivastigmine), NMDA antagonists (e.g., memantine), and free-radical scavengers (e.g., tocopherol). Additional therapeutic agents for Amyotrophic Lateral Sclerosis include, but are not limited to, glutamate pathway antagonists (e.g., riluzole), antispastic agents (e.g., baclofen). Additional therapeutic agents for prion diseases include, but are not limited to, Congo red and its analogs, anthracyclines, amphotericin B and its analogs, sulfated polyanions, and tetrapyrroles. Additional therapeutic agents for type 2 diabetes mellitus include, but are not limited to, sulfonylurea agents (e.g., glipizide, glyburide, glimepiride), meglitinides (e.g., repaglinide, nateglinide), biguanides (e.g., metformin), thiazolidinediones (e.g., pioglitazone, rosiglitazone), dipeptidyl peptidase IV (DPP-4) inhibitors (e.g., sitagliptin), incretin mimetics (e.g., exenatide), amylin analogs (e.g., pramlintide acetate), and alpha-glucosidase inhibitors (e.g., acarbose, miglitol).

In certain embodiments, the present invention provides methods for detecting reduced HSF1 activity (e.g., diminished HSF1 activity, diminished HSF1 protein levels, increased HSF1 Ser303 phosphorylation, increased HSF1 Ser307 phosphorylation, reduced Hsp70 protein levels) within a subject. Indeed, experiments conducted during the course of developing embodiments for the present invention determined that HSF1 levels are progressively lower in muscle of a Huntington's Disease mouse model. As such, in some embodiments, the present invention provides methods for detecting aberrant HSF1 activity in a subject through detecting HSF1 protein levels or HSF1 activity within muscle cells. For example, in some embodiments, a sample is obtained from a subject comprising muscle cells, HSF1 or Hsp70 protein levels within the collected sample is measured, the detected HSF1 protein levels within such a sample is compared with established norms indicating likelihood of developing a condition involving reduced HSF1 activity, severity of a particular condition involving reduced HSF1 activity, improvement of condition over a period of time, etc.

In one aspect, the present invention relates to personalized medicine for patients having a condition involving reduced HSF1 activity (e.g., diminished HSF1 activity, diminished HSF1 protein levels, increased HSF1 Ser303 phosphorylation, increased HSF1 Ser307 phosphorylation), and encompasses the selection of treatment options with the highest likelihood of successful outcome for individual patients. In another aspect, the present invention relates to the use of an assay(s) to predict the treatment outcome, e.g., the likelihood of favorable responses or treatment success, in patients having a condition involving reduced HSF1 activity (e.g., diminished HSF1 activity, diminished HSF1 protein levels, increased HSF1 Ser303 phosphorylation, increased HSF1 Ser307 phosphorylation).

Provided herein are methods of selecting a patient, e.g., human subject for treatment of a condition having reduced HSF1 activity with a CK2 inhibitor, comprising obtaining a biological sample, e.g., muscle cells, from the patient, testing a biological sample from the patient for the presence of HSF1 protein levels and HSF1 activity, and selecting the patient for treatment if the biological sample indicates reduced HSF1 activity. In one embodiment, the methods further comprise administering a therapeutically effective amount of a CK2 inhibitor to the patient if the biological sample indicates reduced HSF1 activity (e.g., diminished HSF1 activity, diminished HSF1 protein levels, increased HSF1 Ser303 phosphorylation, increased HSF1 Ser307 phosphorylation).

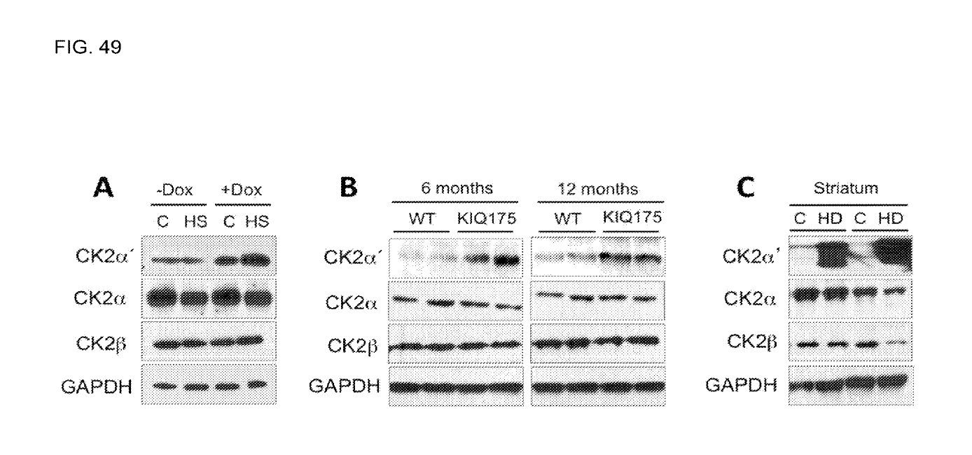

Provided herein are methods of selecting a patient, e.g., human subject for treatment of a condition having reduced HSF1 activity with a CK2 inhibitor, comprising obtaining a biological sample, e.g., muscle cells, from the patient, testing a biological sample from the patient for the presence of increased CK2 levels (see, e.g., Example 7 and FIG. 49), and selecting the patient for treatment if the biological sample further indicates increased CK2 levels. In one embodiment, the methods further comprise administering a therapeutically effective amount of a CK2 inhibitor to the patient if the biological sample indicates increased CK2 levels.

Provided herein are methods of predicting treatment outcomes in a patient having a condition having reduced HSF1 activity (e.g., diminished HSF1 activity, diminished HSF1 protein levels, increased HSF1 Ser303 phosphorylation, increased HSF1 Ser307 phosphorylation), comprising obtaining a biological sample (e.g., muscle cells), from the patient, testing the biological sample from the patient for the presence of reduced HSF1 activity, wherein the detection of reduced HSF1 activity indicates the patient will respond favorably to administration of a therapeutically effective amount of a CK2 inhibitor.

Provided herein are methods of treating a condition characterized with decreased HSF1 activity, comprising administering a therapeutically effective amount of a CK2 inhibitor to a patient, e.g., a human subject, with a condition involving decreased HSF1 activity. In one embodiment, the patient is selected for treatment with the CK2 inhibitor after the patient's cells have been determined to have decreased HSF1 activity (e.g., diminished HSF1 activity, diminished HSF1 protein levels, increased HSF1 Ser303 phosphorylation, increased HSF1 Ser307 phosphorylation). In one embodiment, the method of treating a patient a condition involving decreased HSF1 activity comprises obtaining a biological sample from the patient (e.g., a biological sample comprising muscle cells), determining whether the biological sample contains cells having decreased HSF1 activity, and administering to the patient a therapeutically effective amount of a CK2 inhibitor.

The term “biomarker” as used herein refers to any biological compound, such as a protein, a fragment of a protein, a peptide, a polypeptide, a nucleic acid, etc. that can be detected and/or quantified in a patient in vivo or in a biological sample obtained from a patient. Furthermore, a biomarker can be the entire intact molecule, or it can be a portion or fragment thereof. In one embodiment, the expression level of the biomarker is measured. The expression level of the biomarker can be measured, for example, by detecting the protein or RNA (e.g., mRNA) level of the biomarker. In some embodiments, portions or fragments of biomarkers can be detected or measured, for example, by an antibody or other specific binding agent. In some embodiments, a measurable aspect of the biomarker is associated with a given state of the patient, such as a particular stage of cancer. For biomarkers that are detected at the protein or RNA level, such measurable aspects may include, for example, the presence, absence, or concentration (i.e., expression level) (i.e., activity level) of the biomarker in a patient, or biological sample obtained from the patient. For biomarkers that are detected at the nucleic acid level, such measurable aspects may include, for example, allelic versions of the biomarker or type, rate, and/or degree of mutation of the biomarker, also referred to herein as mutation status.

For biomarkers that are detected based on expression level of protein or RNA, expression level/activity level measured between different phenotypic statuses can be considered different, for example, if the mean or median expression level of the biomarker in the different groups is calculated to be statistically significant. Common tests for statistical significance include, among others, t-test, ANOVA, Kruskal-Wallis, Wilcoxon, Mann-Whitney, Significance Analysis of Microarrays, odds ratio, etc. Biomarkers, alone or in combination, provide measures of relative likelihood that a subject belongs to one phenotypic status or another. Therefore, they are useful, inter alia, as markers for disease and as indicators that particular therapeutic treatment regimens will likely result in beneficial patient outcomes.

In one embodiment of the disclosure, the biomarker is the HSF1 protein. In one embodiment of the disclosure, the measurable aspect of the HSF1 protein is its protein level compared to a cellular control protein, e.g. GAPDH. In one embodiment of the disclosure, the activity level is one that results in diminished HSF1 protein level. In one embodiment of the disclosure, the activity level is one that results in diminished Hsp70 protein level or any of the genes whose expression is activated by HSF1. In one embodiment of the disclosure, the measurable aspect of the HSF1 protein is phosphorylation of the Ser303 amino acid. In one embodiment of the disclosure, the measurable aspect of the HSF1 protein is phosphorylation of the Ser307 amino acid.

Thus, in certain aspects of the disclosure, the biomarker is HSF1 which is differentially present in a subject of one phenotypic status (e.g., a patient having a condition involving diminished HSF1 activity, e.g., Huntington's Disease) as compared with another phenotypic status (e.g., a normal undiseased patient or a patient having cells with normal HSF1 protein levels).

The determination of the expression level or mutation status of a biomarker in a patient can be performed using any of the many methods known in the art. Any method known in the art for quantitating specific proteins and/or detecting HSF1 protein levels in a patient or a biological sample may be used in the methods of the disclosure. Examples include, but are not limited to, PCR (polymerase chain reaction), or RT-PCR, Northern blot, Western blot, ELISA (enzyme linked immunosorbent assay), RIA (radioimmunoassay), gene chip analysis of RNA expression, immunohistochemistry or immunofluorescence (See, e.g., Slagle et al. Cancer 83:1401 (1998)). Certain embodiments of the disclosure include methods wherein biomarker RNA expression (transcription) is determined. Other embodiments of the disclosure include methods wherein protein expression in the biological sample is determined. See, for example, Harlow et al., Antibodies: A Laboratory Manual, Cold Spring Harbor Laboratory, Cold Spring Harbor, N.Y., (1988) and Ausubel et al., Current Protocols in Molecular Biology, John Wiley & Sons, New York 3rd Edition, (1995). For northern blot or RT-PCR analysis, RNA is isolated from the tumor tissue sample using RNAse free techniques. Such techniques are commonly known in the art.

When quantified in a patient in vivo, the expression level of proteins such as HSF1 or Hsp70 may be determined by administering an antibody that binds specifically to HSF1 or Hsp70, respectively, and determining the extent of binding. The antibody may be detectably labeled, e.g., with a radioisotope such as carbon-11, nitrogen-13, oxygen-15, and fluorine-18. The label may then be detected by positron emission tomography (PET).

In one embodiment of the disclosure, a biological sample is obtained from the patient and cells in the biopsy are assayed for determination of biomarker expression/activity level.

In one embodiment of the disclosure, PET imaging is used to determine biomarker expression/activity.

In still another embodiment of the disclosure, expression/activity of proteins encoded by biomarkers are detected by western blot analysis. A western blot (also known as an immunoblot) is a method for protein detection in a given sample of tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are detected using a primary antibody that specifically bind to the protein. The bound antibody can then detected by a secondary antibody that is conjugated with a detectable label (e.g., biotin, horseradish peroxidase or alkaline phosphatase). Detection of the secondary label signal indicates the presence of the protein.

In still another embodiment of the disclosure, the expression/activity of a protein encoded by a biomarker is detected by enzyme-linked immunosorbent assay (ELISA). In one embodiment of the disclosure, “sandwich ELISA” comprises coating a plate with a capture antibody; adding sample wherein any antigen present binds to the capture antibody; adding a detecting antibody which also binds the antigen; adding an enzyme-linked secondary antibody which binds to detecting antibody; and adding substrate which is converted by an enzyme on the secondary antibody to a detectable form. Detection of the signal from the secondary antibody indicates presence of the biomarker antigen protein.

In still another embodiment of the disclosure, the expression of a biomarker is evaluated by use of a gene chip or microarray. Such techniques are within ordinary skill held in the art.

The term “biological sample” as used herein refers any tissue or fluid from a patient that is suitable for detecting a biomarker, such as HSF1 activity level or HSF1 protein level. Examples of useful biological samples include, but are not limited to, biopsied tissues and/or cells, e.g., muscle cells, neurological cells, lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for biomarker expression and/or mutation using any technique known in the art and can be obtained using techniques that are well within the scope of ordinary knowledge of a clinical practioner. In one embodiment of the disclosure, the biological sample comprises blood cells.

### EXPERIMENTAL

The following examples are provided to demonstrate and further illustrate certain preferred embodiments of the present invention and are not to be construed as limiting the scope thereof.

### Example 1

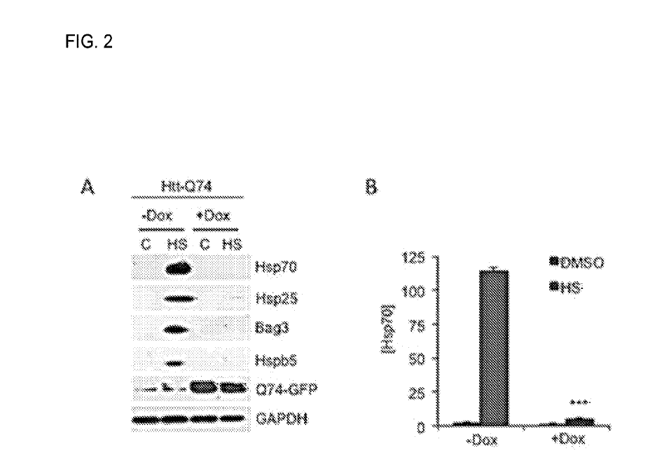

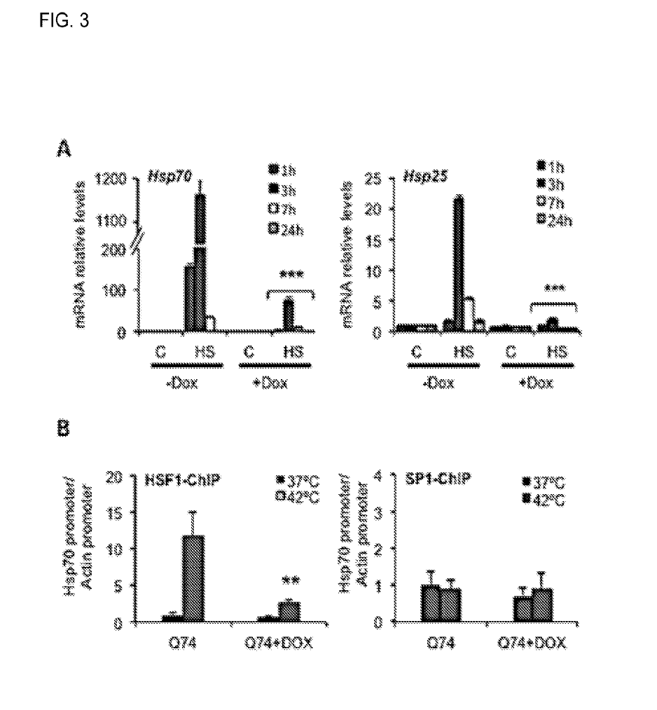

This example demonstrates that HSF1 protein levels are compromised in proteopathies such as neurodegeneration. Increasing HSF1 levels in an Alzheimer's disease mouse model has been shown to have therapeutic benefits in mice. While previous studies in disease models suggest that HSF1 activation has strong potential as a therapeutic target in Huntington's disease (HD), recent work in mouse HD models demonstrates that HSF1 activation is transient and less effective with disease progression (see, e.g., Labbadia, et al., J Clin Invest. 2011; 121(8):3306-3319). It was proposed that HSF1 binding to target gene promoters in vivo is severely compromised due to altered chromatin structure in HD. This interpretation of altered chromatin structure is considered as a significant working model of HSF1 activation in HD cells. Studies in PC12 neuronal precursor cell models of HD recapitulate defective HSF1 activation in the presence of polyQ protein aggregates (FIG. 2 and FIG. 3).

### Example 2

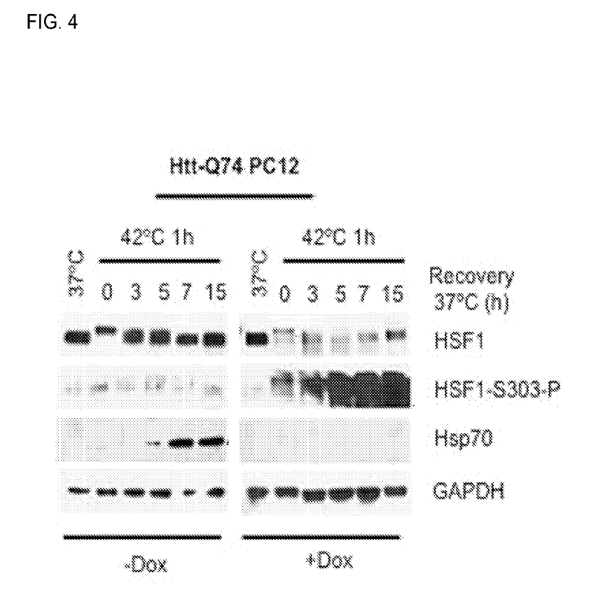

This example demonstrates that HSF1 protein levels are reduced, HSF1 target gene (eg. Hsp70) activation is compromised and HSF1 phosphorylation at Serine 303 is elevated in HD models. Results suggest that HSF1 protein levels, and HSF1 target gene expression (Hsp70) inversely correlate with phosphorylation at Serine 303 (S303) (FIG. 4.).

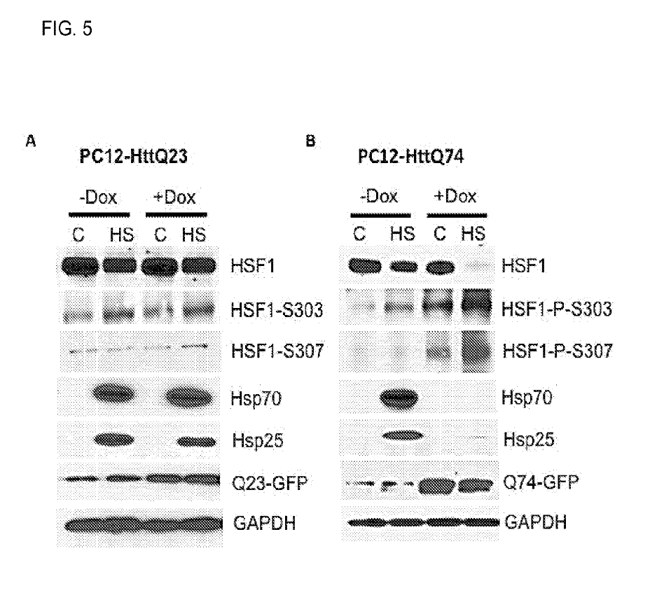

There exist non-pathogenic polyQ repeats in the Huntington's Disease protein (Htt) and pathogenic repeats in Htt. This example shows that HSF1 protein levels are low, HSF1 target gene (Hsp70, Hsp25) activation is low, HSF1 phosphorylation at Serine 303 is elevated and HSF1 phosphorylation at Serine 307 is elevated in cells expressing the disease pathogenic form of Htt (Htt-Q74) but not in the non-disease pathogenic form of Htt (Htt-Q23) (FIG. 5).

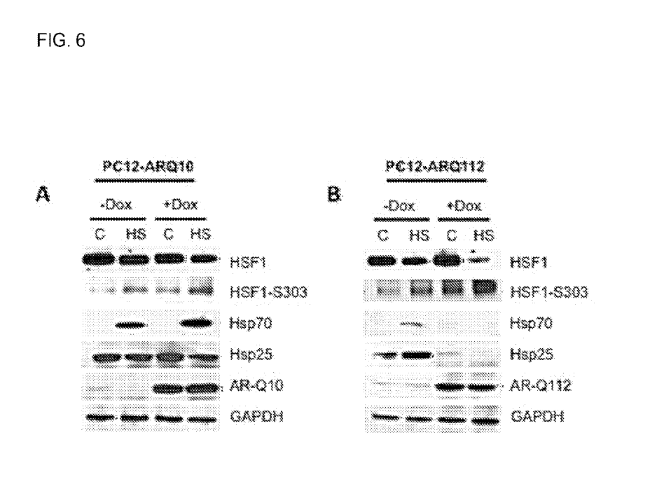

Mutations resulting in long repeats of the amino acid gluatamine (polyQ), are associated with at least 14 neurodegenerative diseases. Huntington's disease is an example here, but additional diseases, such as spinal-bulbar muscular atrophy (SBMA, Kennedy's Disease), involving a poly-glutamine expansion in the Androgen receptor, has similar biology with respect to reduced levels of HSF1 protein, defective HSF1 activation of Hsp70 and Hsp25 gene expression, as well as increased HSF1 phosphorylation at Serine 303 (FIG. 6).

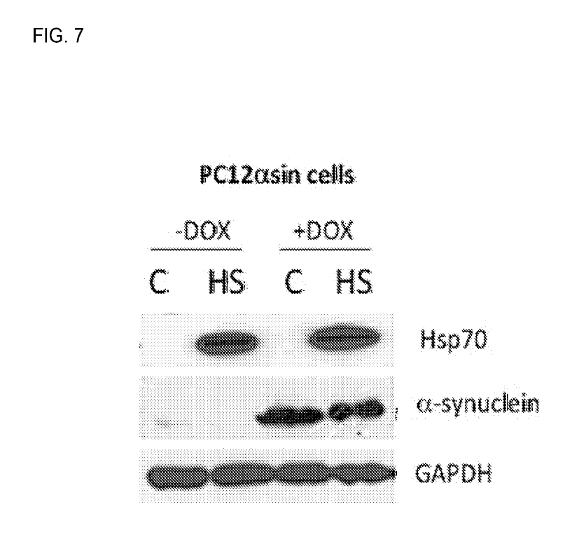

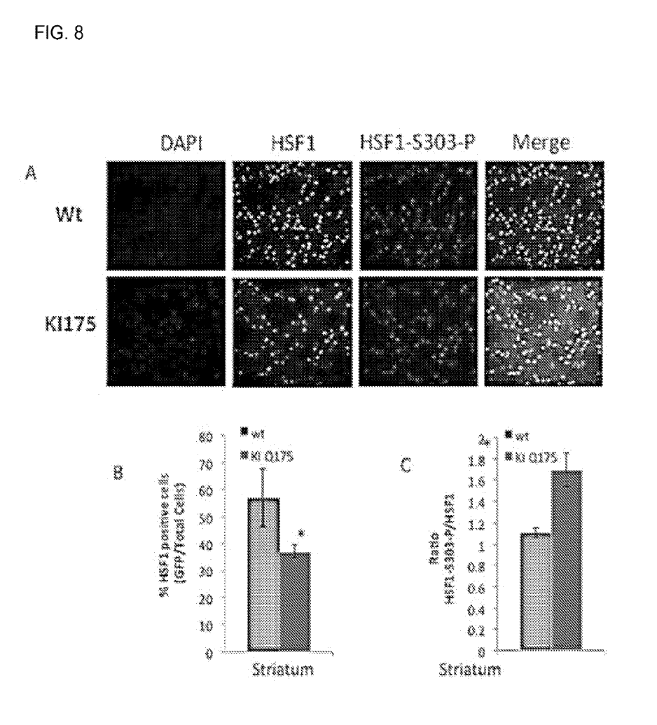

Other diseases with decrease in HSF1 include Tau associated diseases, including but not limited to progressive supranuclear palsy, corticobasal degeneration, Pick's disease, and familial frontotemporal dementia. Data from others show that some forms of Parkinson's Disease (PD) (Xsome 17 associated) have low levels of HFS1, but not all PD. Similarly, some rare forms of ALS may have low level of HSF1 but not all (FIGS. 7, 8 and 9). Prion disease, such as Creutzfeldt-Jakob disease are included into diseases where HSF1 level is decreased and CK2 activity increased (see, e.g., Meggio, et al., 2000 Biochem J. 352(1):191-196).

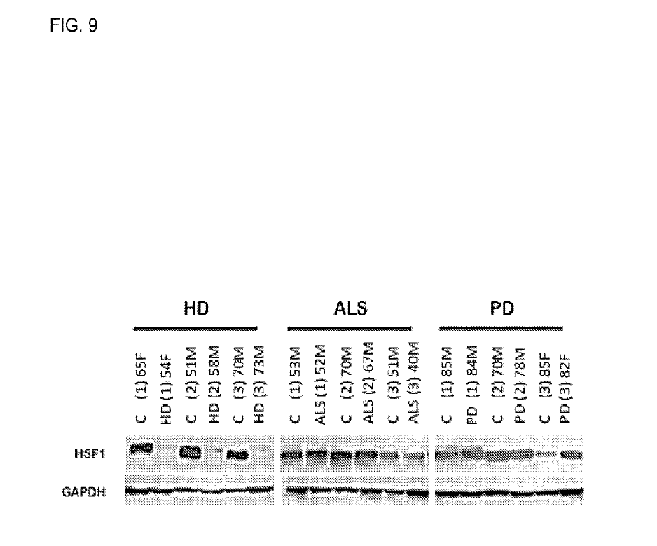

Some neurodegenerative diseases do not have excessive phosphorylation based destruction of HSF1 protein and shut down of cell's stress protection. The measure of HSF1 levels in cells with the use of immunological tools from a sample taken from the patient, e.g. Huntington disease patient′ muscle biopsy, will identify diseases with the lower HSF1 levels. Data shown here show that in HD patient brain samples HSF1 is disappearing compared to age matched controls (FIG. 9), and we have shown that muscle biopsy shown similar pathology. Such experiments demonstrate that muscle biopsy can be used in the diagnosis of HSF1 levels in cells and if HSF1 is found to be low, treatment with CK2 inhibitors will bring the HSF1 levels back and cell viability improves.

### Example 3

This example demonstrates that Casein Kinase Inhibition, pharmacologically, or genetically, elevates HSF1 protein levels, decreases HSF1 phosphorylation and enhances cell survival. Experiments conducted during the course of developing embodiments for the present invention discovered that the use of Casein Kinase 2 (CK2) inhibitors restores HSF1 protein levels, decreases HSF1 phosphorylation at Serine 303, increases protein chaperone expression levels, reduces poly-glutamine protein aggregation and enhances cell viability in cells expressing a pathologic Huntington's Disease protein (Htt-Q74) (FIGS. 10 and 11.)

CK 2 inhibitors were used to rescue cells from protein misfolding associated cell death. This is the first identification of a group of diseases where the HSF1 level is strongly reduced as disease progresses within the cell and where HSF1 levels can be revived with the use of CK2 inhibitors (e.g. emodin, TID43, TBBz, 7,8-dichloro-1,4-dihydro-4-oxo-3-quinolinecarboxylic acid (Cay10577) SB216763 and other CK2 inhibitors) and where restoring HSF1 levels has a therapeutic effect in HD cell models and, prophetically, in human Huntington's Disease. This group of diseases includes, but is not limited to HD, Alzheimer's disease, Cystic Fibrosis and potentially other neurodegenerative diseases or not associated with neurology, including, but not limited to diabetes, cardiac disease, Crohn's disease or any other disease where increasing HSF1 levels may have therapeutic benefit.

### Example 4

This example demonstrates that HSF1 protein levels, and Hsp70 protein levels, are progressively lower in muscle of a Huntington's Disease mouse model (see FIG. 46). Three wild type mice or four KIQ175 Huntinton's Disease mice were used to obtain gastrocnemius muscle biopsies at two or six months of age. Total protein extracts were generated and analyzed by immunoblotting for HSF1 and Hsp70 levels, and GAPDH as a loading control. HSF1 levels, and Hsp70 levels are progressively lower in the KIQ175 mouse, over time.

### Example 5

CK2 inhibitors alone or in conjunction with HSF1 activators such as HSF1A, Hsp90 inhibitors, and all other known HSF1 activators are predicted to have therapeutic efficacy in diseases where elevating the levels of HSF1 could be beneficial. Casein kinase 2 is composed of multiple subunits. Inhibitors that specifically target the CK2 alpha/alpha prime catalytic subunits rescue HSF1 levels in polyQ protein expressing cells. When alpha and alpha prime were tested separately, alpha and alpha prime were the key target to inhibit for HSF1 protein stabilization. In fact, specific CK2 inhibitors for alpha prime inhibition appear to be specific for therapeutic purposes (FIG. 47).

### Example 6

A wild type amino acid sequence for HSF1 (homo sapiens) is provided as follows (see, http: followed by www.ncbi.nlm.nih.gov/protein/NP_005517.1) (SEQ ID NO: 2):

| | |
|---|---|
| 1 | mdlpvgpgaa gpsnvpaflt klwtlvsdpd tdalicwsps gnsfhvfdqg qfakevlpky |
| 61 | fkhnnmasfv rqlnmygfrk vvhieqgglv kperddtefq hpcflrgqeq llenikrkvt |
| 121 | svstlksedi kirqdsvtkl ltdvqlmkgk qecmdsklla mkhenealwr evaslrqkha |
| 181 | qqqkvvnkli qflislvqsn rilgvkrkip lmlndsgsah smpkysrqfs lehvhgsgpy |
| 241 | sapspaysss slyapdavas sgpiisdite lapaspmasp ggsiderpls ssplvrvkee |
| 301 | ppsppqsprv eeaspgrpss vdtllsptal idsilresep apasvtaltd arghtdtegr |
| 361 | ppsppptstp ekclsvacld knelsdhlda mdsnldnlqt mlsshgfsvd tsalldlfsp |
| 421 | svtvpdmslp dldsslasiq ellspqeppr ppeaensspd sgkqlvhyta qplflldpgs |
| 481 | vdtgsndlpv lfelgegsyf segdgfaedp tislltgsep pkakdptvs. |

A wild type amino acid sequence for Htt (homo sapiens) is provided as follows (see, http:// followed by www.ncbi.nlm.nih.gov/protein/P42858.2) (SEQ ID NO: 3):

| | |
|---|---|
| 1 | matleklmka feslksfqqq qqqqqqqqqq qqqqqqqqpp pppppppppq lpqpppqaqp |
| 61 | llpqpqpppp ppppppgpav aeeplhrpkk elsatkkdrv nhclticeni vaqsvrnspe |
| 121 | fqkllgiame lfllcsddae sdvrmvadec lnkvikalmd snlprlqlel ykeikkngap |
| 181 | rslraalwrf aelahlvrpq kcrpylvnll pcltrtskrp eesvqetlaa avpkimasfg |
| 241 | nfandneikv llkafianlk sssptirrta agsavsicqh srrtqyfysw llnvllgllv |
| 301 | pvedehstll ilgvlltlry lvpllqqqvk dtslkgsfgv trkemevsps aeqlvqvyel |
| 361 | tlhhtqhqdh nvvtgalell qqlfrtpppe llqtltavgg igqltaakee sggrsrsgsi |
| 421 | veliagggss cspvlsrkqk gkvllgeeea leddsesrsd vsssaltasv kdeisgelaa |
| 481 | ssgvstpgsa ghdiiteqpr sqhtlqadsv dlascdltss atdgdeedil shsssqvsav |
| 541 | psdpamdlnd gtqasspisd ssqtttegpd savtpsdsse ivldgtdnqy lglqigqpqd |
| 601 | edeeatgilp deaseafrns smalqqahll knmshcrqps dssvdkfvlr deatepgdqe |
| 661 | nkpcrikgdi gqstdddsap lvhcvrllsa sflltggknv lvpdrdvrvs vkalalscvg |
| 721 | aavalhpesf fsklykvpld tteypeeqyv sdilnyidhg dpqvrgatai lcgtlicsil |
| 781 | srsrfhvgdw mgtirtltgn tfsladcipl lrktlkdess vtcklactav rncvmslcss |
| 841 | syselglqli idvltlrnss ywlvrtelle tlaeidfrlv sfleakaenl hrgahhytgl |
| 901 | lklqervlnn vvihllgded prvrhvaaas lirlvpklfy kcdqgqadpv vavardqssv |
| 961 | ylkllmhetq ppshfsvsti triyrgynll psitdvtmen nlsrviaavs helitsttra |
| 1021 | ltfgccealc llstafpvci wslgwhcgvp plsasdesrk sctvgmatmi ltllssawfp |
| 1081 | ldlsahqdal ilagnllaas apkslrsswa seeeanpaat kqeevwpalg dralvpmveq |
| 1141 | lfshllkvin icahvlddva pgpaikaalp sltnppslsp irrkgkekep gegasvplsp |
| 1201 | kkgseasaas rqsdtsgpvt tskssslgsf yhlpsylklh dvlkathany kvtldlqnst |
| 1261 | ekfggflrsa ldvlsqilel atlqdigkcv eeilgylksc fsrepmmatv cvqqllktlf |
| 1321 | gtnlasqfdg lssnpsksqg raqrlgsssv rpglyhycfm apythftqal adaslrnmvq |
| 1381 | aeqendtsgw fdvlqkvstq lktnltsvtk nradknaihn hirlfeplvi kalkqytttt |
| 1441 | cvqlqkqvld llaqlvqlrv nyclldsdqv figfvlkqfe yievgqfres eaiipnifff |
| 1501 | lvllsyeryh skqiigipki iqlcdgimas grkavthaip alqpivhdlf vlrgtnkada |
| 1561 | gkeletqkev vvsmllrliq yhqvlemfil vlqqchkene dkwkrlsrqi adiilpmlak |
| 1621 | qqmhidshea lgvlntlfei lapsslrpvd mllrsmfvtp ntmasvstvq lwisgilail |
| 1681 | rvlisqsted ivlsriqels fspylisctv inrlrdgdst stleehsegk qiknlpeetf |
| 1741 | srfllqlvgi lledivtkql kvemseqqht fycqelgtll mclihifksg mfrritaaat |
| 1801 | rlfrsdgcgg sfytldslnl rarsmitthp alvllwcqil llvnhtdyrw waevqqtpkr |
| 1861 | hslsstklls pqmsgeeeds dlaaklgmcn reivrrgali lfcdyvcqnl hdsehltwli |
| 1921 | vnhiqdlisl sheppvqdfi savhrnsaas glfigaiqsr cenlstptml kktlqclegi |
| 1981 | hlsqsgavlt lyvdrllctp frvlarmvdi lacrrvemll aanlqssmaq lpmeelnriq |
| 2041 | eylqssglaq rhqrlyslld rfrlstmqds lspsppvssh pldgdghvsl etvspdkdwy |
| 2101 | vhlvksqcwt rsdsallega elvnripaed mnafmmnsef nlsllapcls lgmseisggq |
| 2161 | ksalfeaare vtlarvsgtv qqlpavhhvf qpelpaepaa ywsklndlfg daalyqslpt |
| 2221 | laralaqylv vvsklpshlh lppekekdiv kfvvatleal swhliheqip lsldlqagld |
| 2281 | ccclalqlpg lwsvvsstef vthacsliyc vhfileavav qpgeqllspe rrtntpkais |
| 2341 | eeeeevdpnt qnpkyitaac emvaemvesl qsvlalghkr nsgvpafltp llrniiisla |
| 2401 | rlplvnsytr vpplvwklgw spkpggdfgt afpeipvefl qekevfkefi yrintlgwts |
| 2461 | rtqfeetwat llgvlvtqpl vmeqeesppe edtertqinv lavqaitslv lsamtvpvag |
| 2521 | npavscleqq prnkplkald trfgrklsii rgiveqeiqa mvskreniat hhlyqawdpv |
| 2581 | pslspattga lisheklllq inperelgsm syklgqvsih svwlgnsitp lreeewdeee |
| 2641 | eeeadapaps spptspvnsr khragvdihs csqfllelys rwilpsssar rtpailisev |
| 2701 | vrsllvvsdl fternqfelm yvtltelrrv hpsedeilaq ylvpatckaa avlgmdkava |
| 2761 | epvsrllest lrsshlpsrv galhgvlyvl ecdllddtak qlipvisdyl lsnlkgiahc |
| 2821 | vnihsqqhvl vmcatafyli enypldvgpe fsasiiqmcg vmlsgseest psiiyhcalr |
| 2881 | glerlllseq lsrldaeslv klsvdrvnvh sphramaalg lmltcmytgk ekvspgrtsd |
| 2941 | pnpaapdses vivamervsv lfdrirkgfp cearvvaril pqflddffpp qdimnkvige |
| 3001 | flsnqqpypq fmatvvykvf qtlhstgqss mvrdwvmlsl snftqrapva matwslscff |
| 3061 | vsastspwva ailphvisrm gkleqvdvnl fclvatdfyr hqieeeldrr afqsvlevva |
| 3121 | apgspyhrll tclrnvhkvt tc |

### Example 7

This example demonstrates that CK2 activity leads to the inactivation of HSF1 and decreased levels of the HSF1 protein and the HSF1 target genes Hsp70 and Hsp25. Cellular and mouse models of HD, and human HD patient and control samples were used to analyze the levels of the CK2 alpha and alpha′ catalytic subunits to assess changes in disease states. Given that increases in CK2alpha′ protein were observed in cellular and mouse models, and in human HD patients, and that inhibition of CK2 activity leads to corrections in HSF1 levels, Hsp70 expression and enhanced cell viability, the role of CK2-ALPHA′ in inhibiting HSF1 and Hsp70 protein levels were evaluated in wild type mice and in CK2-ALPHA′ knock out mice. A heterozygous loss of function allele for CK2-ALPHA′ was introduced into the KI175 HD mouse background and the progeny mice were evaluated for maintenance of thalamostriatal excitatory synapse formation (defective in HD), corticostriatal syapse formation (not changed in HD), for enhancement of HSF1 and Hsp70 levels and for the prevention of muscle wasting that occurs in HD.

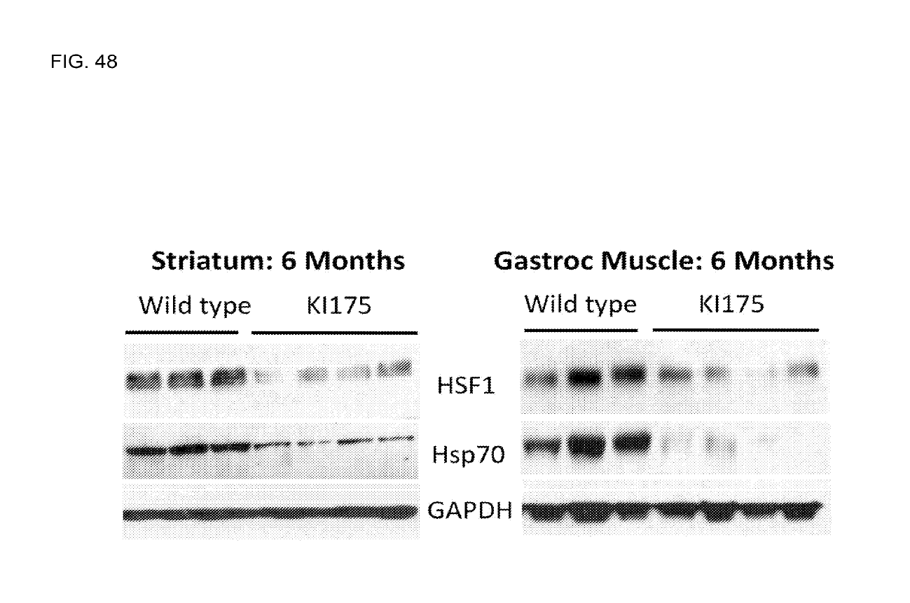

Striatum and gastrocnemius muscle were harvested from 3 WT and 4 KI175 (HD) mice at 6 months and blotted for HSF1, Hsp70 and GAPDH as loading control. FIG. 48 shows that decreased HSF1 and Hsp70 levels were apparent in striatal and skeletal muscle in KI175 HD mouse model.

FIG. 49 shows increased CK2-ALPHA′ in cellular/mouse HD models and HD patient striatum. (A) PC12-HttQ74 cells were cultivated at 37° C. (C) in the presence of Dox for 3 days, exposed to heat shock 1 h at 42° C., followed by recovery period at 37° C. for 7 h (HS). Extracts were immunobloted for CK2 catalytic subunits. (B) Striatal protein samples from Wild type and KIQ175 mice at 6 or 12 months immunoblotted for CK2 subunits. (C) Striatal protein extracts from 2 HD patients and 2 age and sex-matched controls from the Harvard BioBank analyzed by immunoblotting for CK2 subunits. GAPDH was used as loading control.

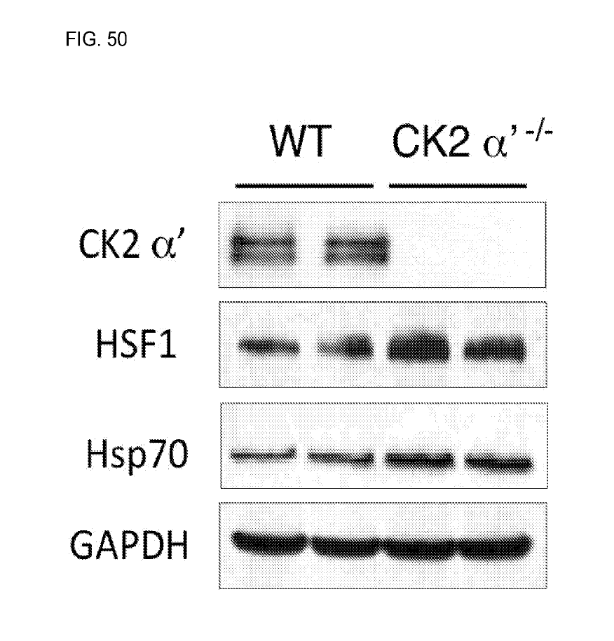

FIG. 50 shows CK2ALPHA′ knock out mice show elevated HSF1 and Hsp70 in mouse striatum. Two independent wild type (WT) or CK2-ALPHA′ knock out (CK2-ALPHA′^{-/-}) mice were evaluated by immunoblotting of striatal tissue for CK2-ALPHA′, HSF1, Hsp70 and GAPDH as loading control.

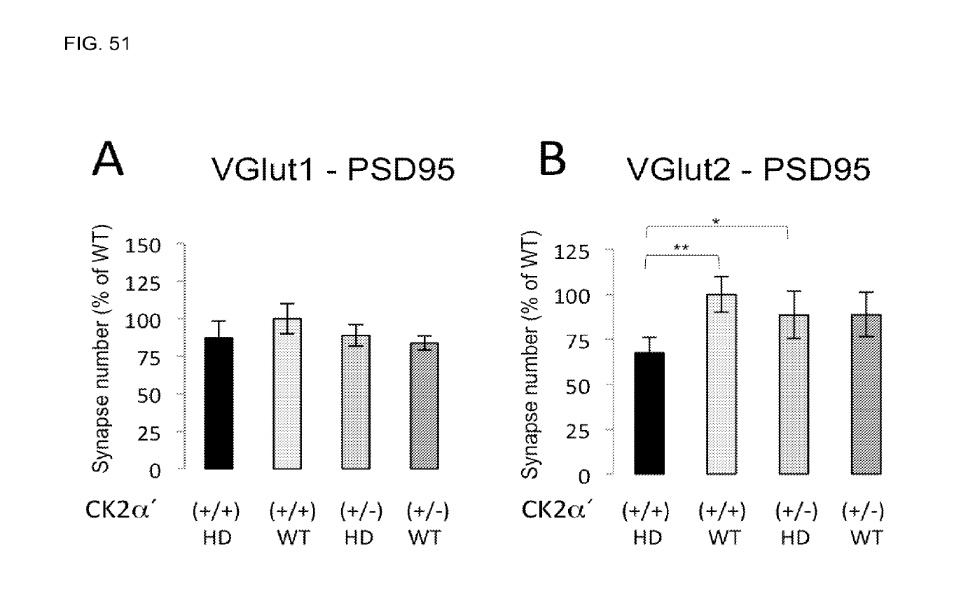

FIG. 51 shows heterozygous loss of CK2-ALPHA′ in KI175 HD enhances thalamostriatal excitatory synapse formation. Mice of the indicated genotype (4/cohort) were analyzed for corticostriatal (VGlut1) and thalamostriatal (VGlut2) excitatory synapse number in the Dorsal striatum at 5 weeks of age.

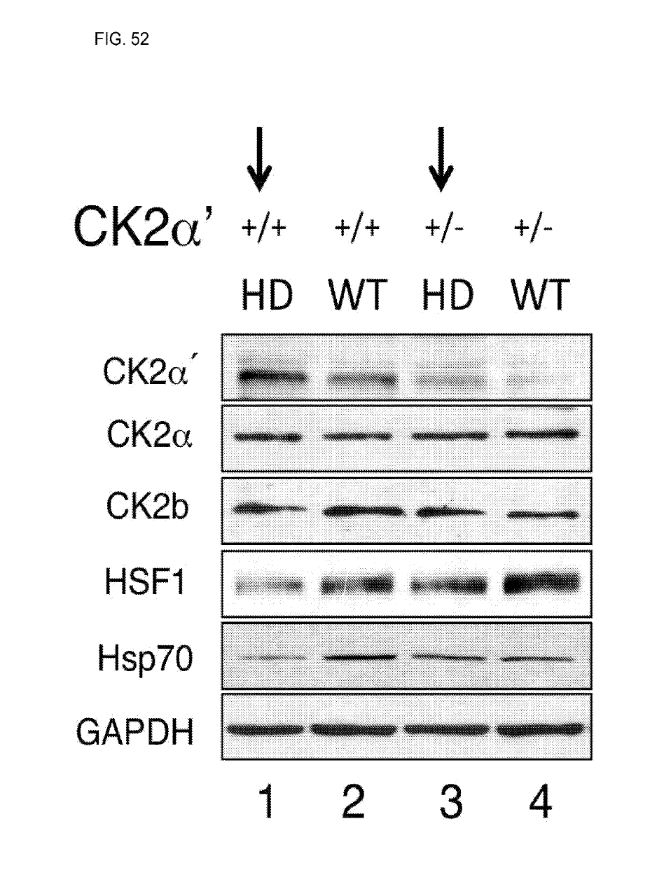

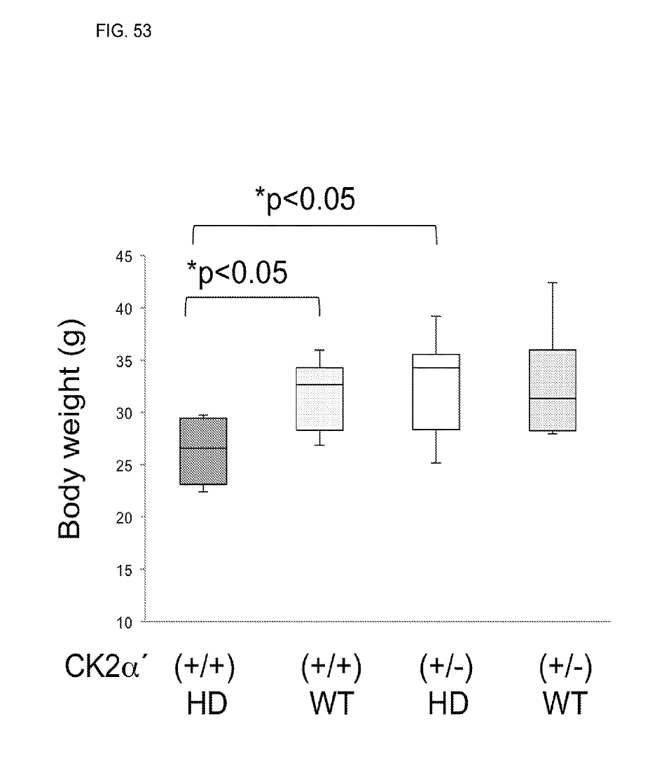

FIG. 52 shows CK2-ALPHA′ reduction increases striatal HSF1, Hsp70 protein levels in KI175 mouse. WT or KIQ175 (HD) mice were crossed to generate mice of the indicated genotype with respect to CK2-ALPHA′ and HD. Striatal extracts from 6 month mice blotted for CK2-ALPHA′, CK2-ALPHA, CK2-BETA, HSF1, Hsp70 and GAPDH. Note the increase in CK2-ALPHA′ and decrease in HSF1 and Hsp70 in the HD mouse compared to the WT mouse (compare lane 1 and 2). Note also the increase in HSF1 and Hsp70 in the CK2-ALPHA′+/−HD mouse, compared to the HD mouse (compare lanes 1 and 3).

FIG. 53 shows CK2-ALPHA′ heterozygous state in the KI175 mouse Huntington's Disease model ameliorates weight loss. Body weight of KIQ175 HD mouse model, WT mice, KIQ175/CK2-ALPHA′(+/−) mice and CK2-ALPHA′(+/−) mice at 6 months of age.

### Example 8

FIG. 54 shows additional compounds identified as CK2 inhibitors. A humanized HSF1 yeast based screen (see, e.g., Neef et al. 2010, PLoS Biology) was established to identify CK2 inhibitors. The humanized HSF1 yeast based screen was used against a library of protein kinase inhibitors and the small molecules shown in FIG. 54 identified. Based on an understanding of the specificity of the screen and the known role of CK2 in yeast and human cells to repress human HSF1, these molecules were construed as having CK2 inhibitory activity.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. (canceled)

2. A method of selecting a patient having a condition ameliorated by elevation of HSF1 protein levels with a CK2 inhibitor, the method comprising:
a) obtaining a biological sample from the patient;
b) determining whether the biological sample contains cells having decreased HSF1 protein levels; and
c) selecting the patient for treatment if the biological sample contains cells having decreased HSF1 protein levels.

3. The method of claim 2, further comprising administering a therapeutically effective amount of the CK2 inhibitor to the patient.

4. (canceled)

5. A method of treating a human patient having a condition ameliorated by elevation of HSF1 protein levels, the method comprising:
a) obtaining a biological sample from the patient;
b) determining whether to biological sample contains cells having aberrant HSF1 protein levels, wherein the biological sample comprises muscle cells and/or neurological cells; and
c) administering a therapeutically effective amount of a CK2 inhibitor to the patient if the biological sample contains cells having aberrant HSF1 protein levels.

6. (canceled)

7. (canceled)

8. The method claim 5, wherein the aberrant HSF1 protein levels is one or more characteristics selected from the group consisting of diminished HSF1 protein levels, diminished Hsp70 protein levels, increased HSF1 Ser303 phosphorylation, and increased HSF1 Ser307 phosphorylation.

9. The method of claim 5, wherein the condition ameliorated by elevation of HSF1 protein is selected from the group consisting of dentatorubropallidoluysian atrophy, Huntington's Disease, spinobulbar muscular atrophy (Kennedy disease), spinocerebellar ataxia type 1, spinocerebellar ataxia type 2, spinocerebellar ataxia type 3 (Machado-Joseph disease), spinocerebellar ataxia type 6, spinocerebellar ataxia type 7, spinocerebellar ataxia type 17, fragile X syndrome, fragile X-associated tremor/ataxia syndrome), fragile XE mental retardation, Friedreich's ataxia, myotonic dystrophy, spinocerebellar ataxia type 8, spinocerebellar ataxia type12, a proteopathy, Alzheimer's disease, glaucoma, tauopathies, fronto-temporal degeneration, familial dementia, Cushing's disease, neurofibromatosis, some lysosomal storage diseases, diabetes, cataracts, cardiac atrial amyloidosis, Parkinson's disease, cystic fibrosis, sickle cell disease, cerebral β-amyloid angiopathy, retinal ganglion cell degeneration in glaucoma, prion diseases, synucleinopathies, tauopahties, frontotemporal lobar degeneration, amyotrophic lateral sclerosis, hereditary cerebral hemorrhage with amyloidosis, cerebral autosomal-dominant arteriopathy with subcortical infarcts and leukoencephalopathy, Alexander disease, seipinopathies, familial amyloidotic neuropathy, senile systemic amyloidosis, serpinopathies, light chain amyloidosis, heavy chain amyloidosis, secondary amyloidosis, aortic medial amyloidosis, ApoAI amyloidosis, ApoAII amyloidosis, ApoAIV amyloidosis, familial amyloidosis of the Finnish type, lysozyme amyloidosis, fibrinogen amyloidosis, dialysis amyloidosis, inclusion body myositis/myopathy, retinitis pigmentosa with rhodopsin mutations, medullary thyroid carcinoma, pituitary prolactinoma, hereditary lattice corneal dystrophy, cutaneous lichen amyloidosis, Mallory bodies, corneal lactoferrin amyloidosis, pulmonary alveolar proteinosis, odontogenic (Pinborg) tumor amyloid, seminal vesical amyloid, cystric fibrosis, and critical illness myopathy.

10. (canceled)

11. (canceled)

12. The method of claim 5, wherein the CK2 inhibitor is selected from the group consisting of TID43, Emodin, TBBz, 7,8-dichloro-1,4-dihydro-4-oxo-3-quinolinecarboxylic acid (Cay10577), resorufin, TBCA, Quinalizarin, AZ285, TID43, TBB, CX-4945, CX-5011, CX-5279, TBI, TBCA, DMAT, CIBG-300, Ellagic Acid, K64/PBIN, K66/TMCB, IQA, Fisetin, Hematein, FLC21, TID46, Quinolone 9, Quinolone 7, TTP22, FNH79, 7-Amino-5-(4-methoxyphenyl)pyrazolo[1,5-a]pyrimidine-3-carbonitrile, 7-(Cyclopropylamino)-5-(2-thienyl)pyrazolo[1,5-a]pyrimidine-3-carbonitrile, 7-(Cyclopropylamino)-5-(4-methoxyphenyl)pyrazolo[1,5-a]pyrimidine-3-carbonitrile, 5-(4-Methoxyphenyl)-7-[(1-methyl-1H-pyrazol-3-yl)amino]pyrazolo[1,5-a]pyrimidine-3-carbonitrile, 7-(Cyclopropylamino)-5-(6-methoxypyridin-3-yl)pyrazolo[1,5-a]pyrimidine-3-carbonitrile, N-(1-(3-cyano-7-(cyclopropylamino)pyrazolo[1,5-a]pyrimidin-5-yl)-1H-indol-6-yl)acetamide, N-(1-(3-cyano-7-(cyclopropylamino)pyrazolo[1,5-a]pyrimidin-5-yl)-1H-indazol-6-yl)acetamide, N-[1-[3-cyano-7-(cyclopropylamino)pyrazolo[1,5-a]pyrimidin-5-yl]pyrrolo[3,2-b]pyridin-6-yl]acetamide, N-[1-[3-cyano-7-(cyclopropylamino)pyrazolo[1,5-a]pyrimidin-5-yl]indolin-6-yl]acetamide, N-{4-[3-cyano-7-(cyclopropylamino)pyrazolo[1,5-a]pyrimidin-5-yl]-3,4-dihydro-2H-1,4-benzoxazin-6-yl}acetamide, N-(1-(3-cyano-7-(cyclopropylamino)pyrazolo[1,5-a]pyrimidin-5-yl)-1,2,3,4-tetrahydroquinolin-7-yl)acetamide, N-(1-(3-cyano-7-(cyclopropylamino)pyrazolo[1,5-a]pyrimidin-5-yl)-5-methyl-1H-indol-6-yl)acetamide, N-(1-(3-cyano-7-(1-methyl-1H-pyrazol-3-ylamino)pyrazolo[1,5-a]pyrimidin-5-yl)-1H-indol-6-yl)acetamide, N-(1-(3-cyano-7-(oxetan-3-ylamino)pyrazolo[1,5-a]pyrimidin-5-yl)-1H-indol-6-yl)acetamide, N-(1-(3-cyano-7-(4-hydroxybutylamino)pyrazolo[1,5-a]pyrimidin-5-yl)-1H-indol-6-yl)acetamide, N-(1-(3-cyano-7-(2-morpholinoethylamino)pyrazolo[1,5-a]pyrimidin-5-yl)-1H-indol-6-yl)acetamide, N-(1-(3-cyano-7-(2-(pyrrolidin-1-yl)ethylamino)pyrazolo[1,5-a]pyrimidin-5-yl)-1H-indol-6-yl)acetamide, N-(1-(3-Cyano-7-(3-(dimethylamino)propylamino)pyrazolo[1,5-a]pyrimidin-5-yl)-1H-indol-6-yl)acetamide, N-(1-(3-Cyano-7-(3-(pyrrolidin-1-yl)propylamino)pyrazolo[1,5-a]pyrimidin-5-yl)-1H-indol-6-yl)acetamide, N-(1-(3-Cyano-7-(cyclopropylamino)pyrazolo[1,5-a]pyrimidin-5-yl)-1H-indol-6-yl)-N-methylacetamide, 7-(Cyclopropylamino)-5-(6-(hydroxymethyl)-1H-indol-1-yl)pyrazolo[1,5-a]pyrimidine-3-carbonitrile, 7-(cyclopropylamino)-5-(6-(methylsulfonylmethyl)-1H-indol-1-yl)pyrazolo[1,5-a]pyrimidine-3-carbonitrile, N-{3-[3-cyano-7-(cyclopropylamino)pyrazolo[1,5-a]pyrimidin-5-yl]-1-methyl-1H-indol-5-yl}acetamide, N-(3-(3-cyano-7-(cyclopropylamino)pyrazolo[1,5-a]pyrimidin-5-yl)-1H-indol-5-yl)acetamide, N-(3-(3-cyano-7-(cyclopropylamino)pyrazolo[1,5-a]pyrimidin-5-yl)-1-(2-hydroxyethyl)-1H-indol-5-yl)acetamide, N-(3-(3-cyano-7-(cyclopropylamino)pyrazolo[1,5-yl)-1-(3-hydroxypropyl)-1H-indol-5-yl)acetamide, Methyl 3-(5-acetamido-3-(3-cyano-7-(cyclopropylamino)pyrazolo[1,5-a]pyrimidin-5-yl)-1H-indol-1-yl)propanoate, 3-(5-Acetamido-3-(3-cyano-7-(cyclopropylamino)pyrazolo[1,5-a]pyrimidin-5-yl)-1H-indol-1-yl)propanoic acid, 7-(cyclopropylamino)-5-(6-nitro-1H-indazol-1-yl)pyrazolo[1,5-a]pyrimidine-3-carbonitrile, 5-(6-Amino-1H-indazol-1-yl)-7-(cyclopropylamino)pyrazolo[1,5-a]pyrimidine-3-carbonitrile, CX-4945, CX-5011, CX-5279, CX-5033 (Kang, et al., 2009 PLoS One 4(8):e6611), 5-oxo-5,6-dihydroindolo-(1,2-a)quinazolin-7-yl]acetic acid (IQA), 4,5,6,7-tetrabromobenzotriazole (TBB), CX-8184, 5-oxo-5,6-dihydroindolo[1,2-a]quinazolin-7-yl)acetic acid, myricetin, quercetin, fisetin, kaempferol, luteolin, apigenin, any of the compounds shown in FIGS. 12-45 and 54, 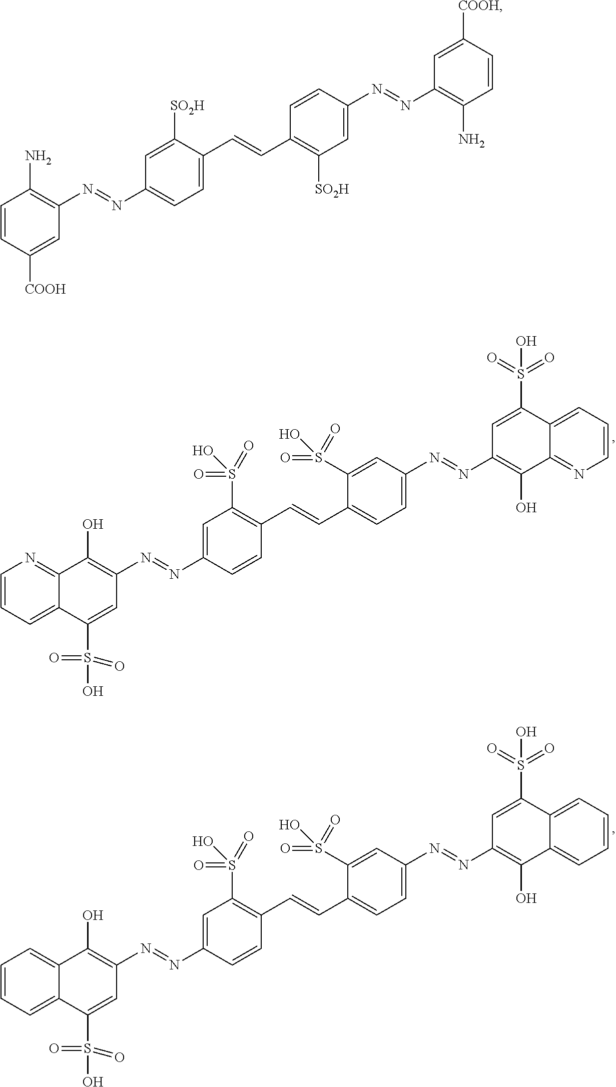
tetrahalogenobenzidazoles, 4,5,6,7-tetrabromo- and 4,5,6,7-tetraiodo-1H-benzimidazoles, and N¹- and 2-S-carboxyalkyl derivatives.

13. The method of claim 5, wherein the CK2 inhibitor targets the CK2-a subunit and/or the CK2a′ subunit of CK2 expressed within cells having diminished HSF1 protein levels.

14. A method of treating a human patient having Alzheimer's Disease and/or Huntington's Disease, the method comprising administering a therapeutically effective amount of a CK2 inhibitor to the patient, wherein administration of the CK2 inhibitor results in one or more of increased HSF1 protein levels, increased Hsp70 protein levels, decreased HSF1 Ser303 phosphorylation, and decreased HSF1 Ser307 phosphorylation levels.

15. (canceled)

16. The method of claim 14, wherein the CK2 inhibitor is selected from the group consisting of TID43, Emodin, TBBz, 7,8-dichloro-1,4-dihydro-4-oxo-3-quinolinecarboxylic acid (Cay10577), resorufin, TBCA, Quinalizarin, AZ285, TID43, TBB, CX-4945, CX-5011, CX-5279, TBI, TBCA, DMAT, CIBG-300, Ellagic Acid, K64/PBIN, K66/TMCB, IQA, Fisetin, Hematein, FLC21, TID46, Quinolone 9, Quinolone 7, TTP22, FNH79, 7-Amino-5-(4-methoxyphenyl)pyrazolo[1,5-a]pyrimidine-3-carbonitrile, 7-(Cyclopropylamino)-5-(2-thienyl)pyrazolo[1,5-a]pyrimidine-3-carbonitrile, 7-(Cyclopropylamino)-5-(4-methoxyphenyl)pyrazolo[1,5-a]pyrimidine-3-carbonitrile, 5-(4-Methoxyphenyl)-7-[(1-methyl-1H-pyrazol-3-yl)amino]pyrazolo[1,5-a]pyrimidine-3-carbonitrile, 7-(Cyclopropylamino)-5-(6-methoxypyridin-3-yl)pyrazolo[1,5-a]pyrimidine-3-carbonitrile, N-(1-(3-cyano-7-(cyclopropylamino)pyrazolo[1,5-a]pyrimidin-5-yl)-1H-indol-6-yl)acetamide, N-(1-(3-cyano-7-(cyclopropylamino)pyrazolo[1,5-a]pyrimidin-5-yl)-1H-indazol-6-yl)acetamide, N-[1-[3-cyano-7-(cyclopropylamino)pyrazolo[1,5-a]pyrimidin-5-yl]pyrrolo[3,2-b]pyridin-6-yl]acetamide, N-[1-[3-cyano-7-(cyclopropylamino)pyrazolo[1,5-a]pyrimidin-5-yl]indolin-6-yl]acetamide, N-{4-[3-cyano-7-(cyclopropylamino)pyrazolo[1,5-a]pyrimidin-5-yl]-3,4-dihydro-2H-1,4-benzoxazin-6-yl}acetamide, N-(1-(3-cyano-7-(cyclopropylamino)pyrazolo[1,5-a]pyrimidin-5-yl)-1,2,3,4-tetrahydroquinolin-7-yl)acetamide, N-(1-(3-cyano-7-(cyclopropylamino)pyrazolo[1,5-a]pyrimidin-5-yl)-5-methyl-1H-indol-6-yl)acetamide, N-(1-(3-cyano-7-(1-methyl-1H-pyrazol-3-ylamino)pyrazolo[1,5-a]pyrimidin-5-yl)-1H-indol-6-yl)acetamide, N-(1-(3-cyano-7-(oxetan-3-ylamino)pyrazolo[1,5-a]pyrimidin-5-yl)-1H-indol-6-yl)acetamide, N-(1-(3-cyano-7-(4-hydroxybutylamino)pyrazolo[1,5-a]pyrimidin-5-yl)-1H-indol-6-yl)acetamide, N-(1-(3-cyano-7-(2-morpholinoethylamino)pyrazolo[1,5-a]pyrimidin-5-yl)-1H-indol-6-yl)acetamide, N-(1-(3-cyano-7-(2-(pyrrolidin-1-yl)ethylamino)pyrazolo[1,5-a]pyrimidin-5-yl)-1H-indol-6-yl)acetamide, N-(1-(3-Cyano-7-(3-(dimethylamino)propylamino)pyrazolo[1,5-a]pyrimidin-5-yl)-1H-indol-6-yl)acetamide, N-(1-(3-Cyano-7-(3-(pyrrolidin-1-yl)propylamino)pyrazolo[1,5-a]pyrimidin-5-yl)-1H-indol-6-yl)acetamide, N-(1-(3-Cyano-7-(cyclopropylamino)pyrazolo[1,5-a]pyrimidin-5-yl)-1H-indol-6-yl)-N-methylacetamide, 7-(Cyclopropylamino)-5-(6-(hydroxymethyl)-1H-indol-1-yl)pyrazolo[1,5-a]pyrimidine-3-carbonitrile, 7-(cyclopropylamino)-5-(6-(methylsulfonylmethyl)-1H-indol-1-yl)pyrazolo[1,5-a]pyrimidine-3-carbonitrile, N-{3-[3-cyano-7-(cyclopropylamino)pyrazolo[1,5-a]pyrimidin-5-yl]-1-methyl-1H-indol-5-yl}acetamide, N-(3-(3-cyano-7-(cyclopropylamino)pyrazolo[1,5-a]pyrimidin-5-yl)-1H-indol-5-yl)acetamide, N-(3-(3-cyano-7-(cyclopropylamino)pyrazolo[1,5-a]pyrimidin-5-yl)-1-(2-hydroxyethyl)-1H-indol-5-yl)acetamide, N-(3-(3-cyano-7-(cyclopropylamino)pyrazolo[1,5-a]pyrimidin-5-yl)-1-(3-hydroxypropyl)-1H-indol-5-yl)acetamide, Methyl 3-(5-acetamido-3-(3-cyano-7-(cyclopropylamino)pyrazolo[1,5-a]pyrimidin-5-yl)-1H-indol-1-yl)propanoate, 3-(5-Acetamido-3-(3-cyano-7-(cyclopropylamino)pyrazolo[1,5-a]pyrimidin-5-yl)-1H-indol-1-yl)propanoic acid, 7-(cyclopropylamino)-5-(6-nitro-1H-indazol-1-yl)pyrazolo[1,5-a]pyrimidine-3-carbonitrile, 5-(6-Amino-1H-indazol-1-yl)-7-(cyclopropylamino)pyrazolo[1,5-a]pyrimidine-3-carbonitrile, CX-4945, CX-5011, CX-5279, CX-5033 (Kang, et al., 2009 PLoS One 4(8):e6611), 5-oxo-5,6-dihydroindolo-(1,2-a)quinazolin-7-yl]acetic acid (IQA), 4,5,6,7-tetrabromobenzotriazole (TBB), CX-8184, 5-oxo-5,6-dihydroindolo[1,2-a]quinazolin-7-yl)acetic acid, myricetin, quercetin, fisetin, kaempferol, luteolin, apigenin, any of the compounds shown in FIGS. 12-45 and 54, 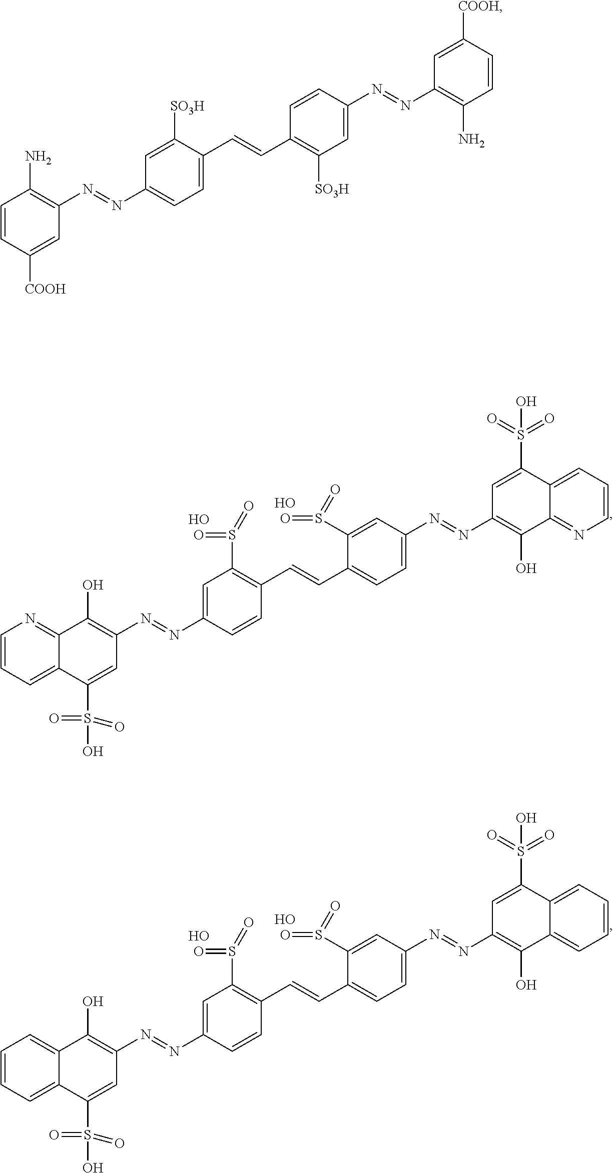
tetrahalogenobenzimidazoles, 4,5,6,7-tetrabromo- and 4,5,6,7-tetraiodo-1H-benzimidazoles and N¹- and 2-S-carboxyalkyl derivatives.

17-24. (canceled)

25. The method of claim 2, wherein the decreased HSF1 protein levels is one or more characteristics selected from the group consisting of diminished HSF1 protein levels, diminished Hsp70 protein levels, increased HSF1 Ser303 phosphorylation, and increased HSF1 Ser307 phosphorylation.

26. The method of claim 2, wherein the condition ameliorated by elevation of HSF1 protein is selected from the group consisting of dentatorubropallidoluysian atrophy, Huntington's Disease, spinobulbar muscular atrophy (Kennedy disease), spinocerebellar ataxia type 1, spinocerebellar ataxia type 2, spinocerebellar ataxia type 3 (Machado-Joseph disease), spinocerebellar ataxia type 6, spinocerebellar ataxia type 7, spinocerebellar ataxia type 17, fragile X syndrome, fragile X-associated tremor/ataxia syndrome), fragile XE mental retardation, Friedreich's ataxia, myotonic dystrophy, spinocerebellar ataxia type 8, spinocerebellar ataxia type12, a proteopathy, Alzheimer's disease, glaucoma, tauopathies, fronto-temporal degeneration, familial dementia, Cushing's disease, neurofibromatosis, some lysosomal storage diseases, diabetes, cataracts, cardiac atrial amyloidosis, Parkinson's disease, cystic fibrosis, sickle cell disease, cerebral β-amyloid angiopathy, retinal ganglion cell degeneration in glaucoma, prion diseases, synucleinopathies, tauopahties, frontotemporal lobar degeneration, amyotrophic lateral sclerosis, hereditary cerebral hemorrhage with amyloidosis, cerebral autosomal-dominant arteriopathy with subcortical infarcts and leukoencephalopathy, Alexander disease, seipinopathies, familial amyloidotic neuropathy, senile systemic amyloidosis, serpinopathies, light chain amyloidosis, heavy chain amyloidosis, secondary amyloidosis, aortic medial amyloidosis, ApoAI amyloidosis, ApoAII amyloidosis, ApoAIV amyloidosis, familial amyloidosis of the Finnish type, lysozyme amyloidosis, fibrinogen amyloidosis, dialysis amyloidosis, inclusion body myositis/myopathy, retinitis pigmentosa with rhodopsin mutations, medullary thyroid carcinoma, pituitary prolactinoma, hereditary lattice corneal dystrophy, cutaneous lichen amyloidosis, Mallory bodies, corneal lactoferrin amyloidosis, pulmonary alveolar proteinosis, odontogenic (Pinborg) tumor amyloid, seminal vesical amyloid, cystric fibrosis, and critical illness myopathy.

27. The method of claim 3, wherein the CK2 inhibitor is selected from the group consisting of TID43, Emodin, TBBz, 7,8-dichloro-1,4-dihydro-4-oxo-3-quinolinecarboxylic acid (Cay10577), resorufin, TBCA, Quinalizarin, AZ285, TID43, TBB, CX-4945, CX-5011, CX-5279, TBI, TBCA, DMAT, CIBG-300, Ellagic Acid, K64/PBIN, K66/TMCB, IQA, Fisetin, Hematein, FLC21, TID46, Quinolone 9, Quinolone 7, TTP22, FNH79, 7-Amino-5-(4-methoxyphenyl)pyrazolo[1,5-a]pyrimidine-3-carbonitrile, 7-(Cyclopropylamino)-5-(2-thienyl)pyrazolo[1,5-a]pyrimidine-3-carbonitrile, 7-(Cyclopropylamino)-5-(4-methoxyphenyl)pyrazolo[1,5-a]pyrimidine-3-carbonitrile, 5-(4-Methoxyphenyl)-7-[(1-methyl-1H-pyrazol-3-yl)amino]pyrazolo[1,5-a]pyrimidine-3-carbonitrile, 7-(Cyclopropylamino)-5-(6-methoxypyridin-3-yl)pyrazolo[1,5-a]pyrimidine-3-carbonitrile, N-(1-(3-cyano-7-(cyclopropylamino)pyrazolo[1,5-a]pyrimidin-5-yl)-1H-indol-6-yl)acetamide, N-(1-(3-cyano-7-(cyclopropylamino)pyrazolo[1,5-a]pyrimidin-5-yl)-1H-indazol-6-yl)acetamide, N-[1-[3-cyano-7-(cyclopropylamino)pyrazolo[1,5-a]pyrimidin-5-yl]pyrrolo[3,2-b]pyridin-6-yl]acetamide, N-[1-[3-cyano-7-(cyclopropylamino)pyrazolo[1,5-a]pyrimidin-5-yl]indolin-6-yl]acetamide, N-{4-[3-cyano-7-(cyclopropylamino)pyrazolo[1,5-a]pyrimidin-5-yl]-3,4-dihydro-2H-1,4-benzoxazin-6-yl}acetamide, N-(1-(3-cyano-7-(cyclopropylamino)pyrazolo[1,5-a]pyrimidin-5-yl)-1,2,3,4-tetrahydroquinolin-7-yl)acetamide, N-(1-(3-cyano-7-(cyclopropylamino)pyrazolo[1,5-a]pyrimidin-5-yl)-5-methyl-1H-indol-6-yl)acetamide, N-(1-(3-cyano-7-(1-methyl-1H-pyrazol-3-ylamino)pyrazolo[1,5-a]pyrimidin-5-yl)-1H-indol-6-yl)acetamide, N-(1-(3-cyano-7-(oxetan-3-ylamino)pyrazolo[1,5-a]pyrimidin-5-yl)-1H-indol-6-yl)acetamide, N-(1-(3-cyano-7-(4-hydroxybutylamino)pyrazolo[1,5-a]pyrimidin-5-yl)-1H-indol-6-yl)acetamide, N-(1-(3-cyano-7-(2-morpholinoethylamino)pyrazolo[1,5-a]pyrimidin-5-yl)-1H-indol-6-yl)acetamide, N-(1-(3-cyano-7-(2-(pyrrolidin-1-yl)ethylamino)pyrazolo[1,5-a]pyrimidin-5-yl)-1H-indol-6-yl)acetamide, N-(1-(3-Cyano-7-(3-(dimethylamino)propylamino)pyrazolo[1,5-a]pyrimidin-5-yl)-1H-indol-6-yl)acetamide, N-(1-(3-Cyano-7-(3-(pyrrolidin-1-yl)propylamino)pyrazolo[1,5-a]pyrimidin-5-yl)-1H-indol-6-yl)acetamide, N-(1-(3-Cyano-7-(cyclopropylamino)pyrazolo[1,5-a]pyrimidin-5-yl)-1H-indol-6-yl)-N-methylacetamide, 7-(Cyclopropylamino)-5-(6-(hydroxymethyl)-1H-indol-1-yl)pyrazolo[1,5-a]pyrimidine-3-carbonitrile, 7-(cyclopropylamino)-5-(6-(methylsulfonylmethyl)-1H-indol-1-yl)pyrazolo[1,5-a]pyrimidine-3-carbonitrile, N-{3-[3-cyano-7-(cyclopropylamino)pyrazolo[1,5-a]pyrimidin-5-yl]-1-methyl-1H-indol-5-yl}acetamide, N-(3-(3-cyano-7-(cyclopropylamino)pyrazolo[1,5-a]pyrimidin-5-yl)-1H-indol-5-yl)acetamide, N-(3-(3-cyano-7-(cyclopropylamino)pyrazolo[1,5-a]pyrimidin-5-yl)-1-(2-hydroxyethyl)-1H-indol-5-yl)acetamide, N-(3-(3-cyano-7-(cyclopropylamino)pyrazolo[1,5-a]pyrimidin-5-yl)-1-(3-hydroxypropyl)-1H-indol-5-yl)acetamide, Methyl 3-(5-acetamido-3-(3-cyano-7-(cyclopropylamino)pyrazolo[1,5-a]pyrimidin-5-yl)-1H-indol-1-yl)propanoate, 3-(5-Acetamido-3-(3-cyano-7-(cyclopropylamino)pyrazolo[1,5-a]pyrimidin-5-yl)-1H-indol-1-yl)propanoic acid, 7-(cyclopropylamino)-5-(6-nitro-1H-indazol-1-yl)pyrazolo[1,5-a]pyrimidine-3-carbonitrile, 5-(6-Amino-1H-indazol-1-yl)-7-(cyclopropylamino)pyrazolo[1,5-a]pyrimidine-3-carbonitrile, CX-4945, CX-5011, CX-5279, CX-5033 (Kang, et al., 2009 PLoS One 4(8):e6611), 5-oxo-5,6-dihydroindolo-(1,2-a)quinazolin-7-yl]acetic acid (IQA), 4,5,6,7-tetrabromobenzotriazole (TBB), CX-8184, 5-oxo-5,6-dihydroindolo[1,2-a]quinazolin-7-yl)acetic acid, myricetin, quercetin, fisetin, kaempferol, luteolin, apigenin, any of the compounds shown in FIGS. 12-45 and 54, 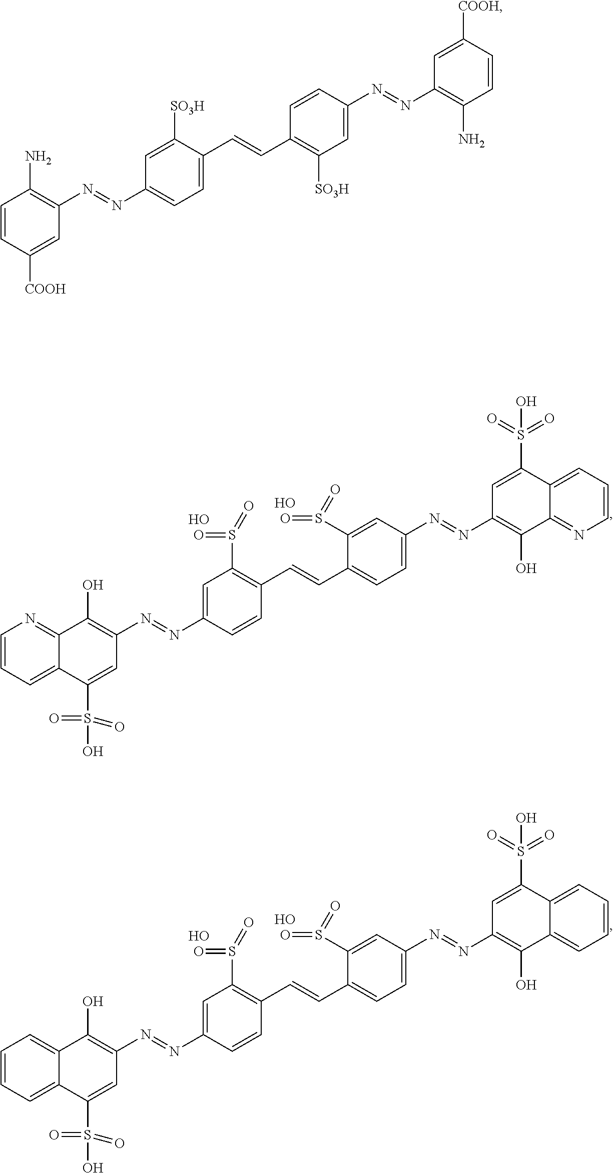
tetrahalogenobenzimidazoles, 4,5,6,7-tetrabromo- and 4,5,6,7-tetraiodo-1H-benzimidazoles, and N¹- and 2-S-carboxyalkyl derivatives.

28. The method of claim 3, wherein the CK2 inhibitor targets the CK2-a subunit and/or the CK2a′ subunit of CK2 expressed within cells having diminished HSF1 protein levels.


SEQUENCE LISTING (4 sequences)

SEQ ID NO: 1 - DNA, 15 bp, Artificial sequence
  Synthetic
ngaannttcnngaan

SEQ ID NO: 2 - protein, 529 aa, Homo sapiens
MDLPVGPGAAGPSNVPAFLTKLWTLVSDPDTDALICWSPSGNSFHVFDQGQFAKEVLPKYFKHNNMASFVRQLNMYGFRKVVHIEQGGLVKPERDDTEFQHPCFLRGQEQLLENIKRKVTSVSTLKSEDIKIRQDSVTKLLTDVQLMKGKQECMDSKLLAMKHENEALWREVASLRQKHAQQQKVVNKLIQFLISLVQSNRILGVKRKIPLMLNDSGSAHSMPKYSRQFSLEHVHGSGPYSAPSPAYSSSSLYAPDAVASSGPIISDITELAPASPMASPGGSIDERPLSSSPLVRVKEEPPSPPQSPRVEEASPGRPSSVDTLLSPTALIDSILRESEPAPASVTALTDARGHTDTEGRPPSPPPTSTPEKCLSVACLDKNELSDHLDAMDSNLDNLQTMLSSHGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 3 - protein, 3142 aa, Homo sapiens
MATLEKLMKAFESLKSFQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 4 - protein, 18 aa, Artificial sequence
  Synthetic
GTIIKENIGVSYDEYRYR